SEQUENCE LISTING (40 sequences)

SEQ ID NO: 1 - protein, 301 aa, Mycobacterium tuberculosis
VVAVTYSPGPHLERFLASLSLATERPVSVLLADNGSTDGTPQAAVQRYPNVRLLPTGANLGYGTAVNRTIAQLGEMAGDAGEPWVDDWVIVANPDVQWGPGSIDALLDAASRWPRAGALGPLIRDPDGSVYPSARQMPSLIRGGMHAVLGPFWPRNPWTTAYRQERLEPSERPVGWLSGSCLLVRRSAFGQVGGFDERYFMYMEDVDLGDRLGKAGWLSVYVPSAEVLHHKAHSTGRDPASHLAAHHKSTYIFLADRHSGWWRAPLRWTLRGSLALRSHLMVRSSLRRSRRRKLKLVEGRH

SEQ ID NO: 2 - protein, 99 aa, Mycobacterium tuberculosis
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 3 - protein, 390 aa, Mycobacterium tuberculosis
VDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAG

SEQ ID NO: 4 - protein, 99 aa, Mycobacterium tuberculosis
VSFVMAYPEMLAAAADTLQSIGATTVASNAAAAAPTTGVVPPAADEVSALTAAHFAAHAAMYQSVSARAAAIHDQFVATLASSASSYAATEVANAAAAS

SEQ ID NO: 5 - DNA, 903 bp, synthetic construct
  source 1..903: mol_type = other DNA
gtggtggccgtgacctacagccccggcccccacctggagagattcctggctagcctgagcctggccaccgaaagacccgtgtccgtgcttttggccgacaacggcagtacggacggcaccccccaagccgccgtgcagagataccccaacgtgagactgctgcccaccggcgccaacctgggctacggcaccgccgtgaatagaacgatagctcagctgggcgagatggccggagacgccggtgaaccctgggtggacgattgggtcatagtggcgaatcccgacgtgcaatggggtcccggcagcatcgatgccctgctggatgccgctagcagatggccccgcgctggtgccctgggaccgttaattcgggaccccgacggcagcgtgtaccctagcgctagacagatgcctagcctgatcagaggcggcatgcacgccgtgctgggccccttctggcctagaaacccctggaccaccgcctacagacaagagagactggagcctagcgaaagacccgtgggctggctgtcgggctcctgcctgctggtgagaagaagcgccttcggccaagtgggcggcttcgacgagagatacttcatgtacatggaggacgtggacctgggcgacagactgggcaaggccggctggctaagcgtttacgtgcctagcgccgaggtgctgcaccacaaggcccacagcaccggccgggaccccgctagccacctggccgcccaccacaagagcacctacatcttcctggccgacagacacagcggctggtggagagcccccctgagatggaccctgagaggcagcctggccctgagaagccacctgatggtgagaagcagcctgagaagaagcagaagaagaaagctgaagctggtggagggcagacac

SEQ ID NO: 6 - DNA, 297 bp, synthetic construct
  source 1..297: mol_type = other DNA
gccgagatgaaaaccgacgccgccaccctggcccaagaggccggcaacttcgagagaatcagcggcgacctcaagacacagatcgaccaagtggagagcacggctggcagcctgcaagggcaatggagaggcgcagccggtactgccgcacaagccgccgtggtgagattccaagaggccgccaacaagcagaagcaagagctggacgagatcagcaccaacatcagacaagccggcgtgcagtacagcagagccgacgaggagcagcagcaagccctgagctctcagatgggcttc

SEQ ID NO: 7 - DNA, 1170 bp, synthetic construct
  source 1..1170: mol_type = other DNA
gtggacttcggcgccctgccccccgagataaactccgctagaatgtacgccggcccgggaagcgctagcttggtcgcagccgcacagatgtgggacagcgtggctagcgacctgttcagcgccgctagcgcctttcagtcggtggtgtggggacttactgtcggcagctggatcggcagttcggctggcttgatggtggccgcggcgtcgccctatgtggcctggatgagcgtgactgccgggcaagccgaactgactgcggctcaagtgcgtgtggccgcggctgcctatgaaaccgcgtacggccttacagttccgcctcccgtgatcgccgagaacagagccgagctgatgatcctgatcgccaccaacctgctggggcagaacacccccgccatcgccgtgaacgaggccgagtacggcgagatgtgggctcaagatgctgcagcgatgttcggatacgccgccgccacggcgaccgccaccgccaccttactgcccttcgaagaagcccccgagatgacaagcgccggcggcttattagagcaagccgcagccgtggaggaggctagcgacacggccgctgccaatcagctgatgaacaatgtgccccaagccctacaacaactggcgcagcccacccaaggcacaaccccaagcagcaagctgggcggcctgtggaaaaccgtgagcccccacagaagccccatcagcaacatggtgagcatggccaacaaccacatgagcatgacaaacagtggcgtgagcatgaccaatacgctcagcagcatgctgaagggcttcgcgccggccgccgctgcacaagctgttcagactgcggcgcagaacggggtgagagcaatgagcagcctgggctcaagccttggcagctctggcttaggcggcggtgtggcagccaacctgggcagagccgcctcagttgggagcctgtccgtgcctcaagcctgggcagcggcgaatcaagctgtcacccccgccgctagagccctgcccctgacaagcctgactagtgcggcagagagaggccccggccaaatgctcggtggcttaccggtagggcaaatgggggcaagagcgggaggcggtctttccggcgtgctgagagtgccccctagaccctacgtgatgccccacagccccgccgccggc

SEQ ID NO: 8 - DNA, 297 bp, synthetic construct
  source 1..297: mol_type = other DNA
gtgtctttcgtgatggcctaccccgaaatgctagccgccgccgcagataccctgcagagcatcggcgcaaccacagttgcaagcaatgctgctgctgccgcaccgactaccggcgtggtgccccccgcggcggatgaggtgagcgccctaaccgccgcccatttcgccgcacacgccgcgatgtatcagagcgtgagcgctagagccgccgccatccacgatcagttcgtggccaccctggctagcagcgctagcagctacgccgccaccgaggtggccaacgcggccgccgctagc

SEQ ID NO: 9 - DNA, 18 bp, synthetic construct
  source 1..18: mol_type = other DNA
ggcggatctggcggcagc

SEQ ID NO: 10 - DNA, 24 bp, synthetic construct
  source 1..24: mol_type = other DNA
ggcggcggcagcggcggcggatct

SEQ ID NO: 11 - DNA, 30 bp, synthetic construct
  source 1..30: mol_type = other DNA
ggcggcggcggcagcggcggaggcggatct

SEQ ID NO: 12 - DNA, 45 bp, synthetic construct
  source 1..45: mol_type = other DNA
ggcggaggcggatctggcggaggcggcagcggcggcggcggcagc

SEQ ID NO: 13 - DNA, 24 bp, synthetic construct
  source 1..24: mol_type = other DNA
ggcggcggcggcggaggcggtggc

SEQ ID NO: 14 - DNA, 18 bp, synthetic construct
  source 1..18: mol_type = other DNA
ggcggcggcggcggaggc

SEQ ID NO: 15 - DNA, 12 bp, synthetic construct
  source 1..12: mol_type = other DNA
ggcggcggcggc

SEQ ID NO: 16 - DNA, 63 bp, synthetic construct
  source 1..63: mol_type = other DNA
ggatctggcgagggcagaggcagcctgctgacctgcggcgacgtggaagagaaccccggccct

SEQ ID NO: 17 - DNA, 66 bp, synthetic construct
  source 1..66: mol_type = other DNA
ggatctggcgccaccaacttcagcctgctgaagcaggccggcgacgtggaagagaaccccggccct

SEQ ID NO: 18 - DNA, 75 bp, synthetic construct
  source 1..75: mol_type = other DNA
ggatctggcgtgaagcagaccctgaacttcgacctgctgaagctggccggcgatgtggagagcaaccccggccct

SEQ ID NO: 19 - DNA, 69 bp, synthetic construct
  source 1..69: mol_type = other DNA
ggatctggccagtgcaccaactacgccctgctgaagctggccggcgacgtggagagcaaccccggccct

SEQ ID NO: 20 - protein, 37 aa, Mycobacterium tuberculosis
MSLRLVSPIKAFADGIVAVAIAVVLMFGLANTPRAVA

SEQ ID NO: 21 - DNA, 111 bp, synthetic construct
  source 1..111: mol_type = other DNA
atgagcctgcggctggtgtctcccatcaaggccttcgccgacggcatcgtggccgtggctatcgccgtggtgctgatgttcggcctggccaacacccctagagccgtcgct

SEQ ID NO: 22 - protein, 29 aa, Mycobacterium tuberculosis
MKLTTMIKTAVAVVAMAAIATFAAPVALA

SEQ ID NO: 23 - DNA, 87 bp, synthetic construct
  source 1..87: mol_type = other DNA
atgaagctgaccaccatgatcaagaccgccgtggctgtggtcgccatggccgctatcgccacattcgccgcccctgtggccctggcc

SEQ ID NO: 24 - protein, 26 aa, Mycobacterium tuberculosis
MKGRSALLRALWIAALSFGLGGVAVA

SEQ ID NO: 25 - DNA, 78 bp, synthetic construct
  source 1..78: mol_type = other DNA
atgaagggcagatctgccctgctgcgggccctgtggatcgccgctctgagcttcggcctgggcggagtggccgtggcc

SEQ ID NO: 26 - protein, 39 aa, Mycobacterium tuberculosis
MHQVDPNLTRRKGRLAALAIAAMASASLVTVAVPATANA

SEQ ID NO: 27 - DNA, 117 bp, synthetic construct
  source 1..117: mol_type = other DNA
atgcaccaggtcgaccccaacctgaccagacggaagggcagactggccgccctggccatcgccgctatggccagcgcctctctggtgaccgtggccgtgcctgctacagccaacgcc

SEQ ID NO: 28 - protein, 20 aa, Homo sapiens
MYRMQLLSCIALSLALVTNS

SEQ ID NO: 29 - DNA, 60 bp, synthetic construct
  source 1..60: mol_type = other DNA
atgtacagaatgcagctgctgagctgcatcgccctgagcctggccctggtgaccaacagc

SEQ ID NO: 30 - DNA, 2793 bp, synthetic construct
  source 1..2793: mol_type = other DNA
atggtgtctttcgtgatggcctaccccgaaatgctagccgccgccgcagataccctgcagagcatcggcgcaaccacagttgcaagcaatgctgctgctgccgcaccgactaccggcgtggtgccccccgcggcggatgaggtgagcgccctaaccgccgcccatttcgccgcacacgccgcgatgtatcagagcgtgagcgctagagccgccgccatccacgatcagttcgtggccaccctggctagcagcgctagcagctacgccgccaccgaggtggccaacgcggccgccgctagcggcggcggcggcagcggcggaggcggatctgtggacttcggcgccctgccccccgagataaactccgctagaatgtacgccggcccgggaagcgctagcttggtcgcagccgcacagatgtgggacagcgtggctagcgacctgttcagcgccgctagcgcctttcagtcggtggtgtggggacttactgtcggcagctggatcggcagttcggctggcttgatggtggccgcggcgtcgccctatgtggcctggatgagcgtgactgccgggcaagccgaactgactgcggctcaagtgcgtgtggccgcggctgcctatgaaaccgcgtacggccttacagttccgcctcccgtgatcgccgagaacagagccgagctgatgatcctgatcgccaccaacctgctggggcagaacacccccgccatcgccgtgaacgaggccgagtacggcgagatgtgggctcaagatgctgcagcgatgttcggatacgccgccgccacggcgaccgccaccgccaccttactgcccttcgaagaagcccccgagatgacaagcgccggcggcttattagagcaagccgcagccgtggaggaggctagcgacacggccgctgccaatcagctgatgaacaatgtgccccaagccctacaacaactggcgcagcccacccaaggcacaaccccaagcagcaagctgggcggcctgtggaaaaccgtgagcccccacagaagccccatcagcaacatggtgagcatggccaacaaccacatgagcatgacaaacagtggcgtgagcatgaccaatacgctcagcagcatgctgaagggcttcgcgccggccgccgctgcacaagctgttcagactgcggcgcagaacggggtgagagcaatgagcagcctgggctcaagccttggcagctctggcttaggcggcggtgtggcagccaacctgggcagagccgcctcagttgggagcctgtccgtgcctcaagcctgggcagcggcgaatcaagctgtcacccccgccgctagagccctgcccctgacaagcctgactagtgcggcagagagaggccccggccaaatgctcggtggcttaccggtagggcaaatgggggcaagagcgggaggcggtctttccggcgtgctgagagtgccccctagaccctacgtgatgccccacagccccgccgccggcggatctggcgagggcagaggcagcctgctgacctgcggcgacgtggaagagaaccccggccctgtggtggccgtgacctacagccccggcccccacctggagagattcctggctagcctgagcctggccaccgaaagacccgtgtccgtgcttttggccgacaacggcagtacggacggcaccccccaagccgccgtgcagagataccccaacgtgagactgctgcccaccggcgccaacctgggctacggcaccgccgtgaatagaacgatagctcagctgggcgagatggccggagacgccggtgaaccctgggtggacgattgggtcatagtggcgaatcccgacgtgcaatggggtcccggcagcatcgatgccctgctggatgccgctagcagatggccccgcgctggtgccctgggaccgttaattcgggaccccgacggcagcgtgtaccctagcgctagacagatgcctagcctgatcagaggcggcatgcacgccgtgctgggccccttctggcctagaaacccctggaccaccgcctacagacaagagagactggagcctagcgaaagacccgtgggctggctgtcgggctcctgcctgctggtgagaagaagcgccttcggccaagtgggcggcttcgacgagagatacttcatgtacatggaggacgtggacctgggcgacagactgggcaaggccggctggctaagcgtttacgtgcctagcgccgaggtgctgcaccacaaggcccacagcaccggccgggaccccgctagccacctggccgcccaccacaagagcacctacatcttcctggccgacagacacagcggctggtggagagcccccctgagatggaccctgagaggcagcctggccctgagaagccacctgatggtgagaagcagcctgagaagaagcagaagaagaaagctgaagctggtggagggcagacacggcggcggcggcggaggcgccgagatgaaaaccgacgccgccaccctggcccaagaggccggcaacttcgagagaatcagcggcgacctcaagacacagatcgaccaagtggagagcacggctggcagcctgcaagggcaatggagaggcgcagccggtactgccgcacaagccgccgtggtgagattccaagaggccgccaacaagcagaagcaagagctggacgagatcagcaccaacatcagacaagccggcgtgcagtacagcagagccgacgaggagcagcagcaagccctgagctctcagatgggcttctgataatgatag

SEQ ID NO: 31 - DNA, 2907 bp, synthetic construct
  source 1..2907: mol_type = other DNA
atgcaccaggtcgaccccaacctgaccagacggaagggcagactggccgccctggccatcgccgctatggccagcgcctctctggtgaccgtggccgtgcctgctacagccaacgccgtgtctttcgtgatggcctaccccgaaatgctagccgccgccgcagataccctgcagagcatcggcgcaaccacagttgcaagcaatgctgctgctgccgcaccgactaccggcgtggtgccccccgcggcggatgaggtgagcgccctaaccgccgcccatttcgccgcacacgccgcgatgtatcagagcgtgagcgctagagccgccgccatccacgatcagttcgtggccaccctggctagcagcgctagcagctacgccgccaccgaggtggccaacgcggccgccgctagcggcggcggcggcagcggcggaggcggatctgtggacttcggcgccctgccccccgagataaactccgctagaatgtacgccggcccgggaagcgctagcttggtcgcagccgcacagatgtgggacagcgtggctagcgacctgttcagcgccgctagcgcctttcagtcggtggtgtggggacttactgtcggcagctggatcggcagttcggctggcttgatggtggccgcggcgtcgccctatgtggcctggatgagcgtgactgccgggcaagccgaactgactgcggctcaagtgcgtgtggccgcggctgcctatgaaaccgcgtacggccttacagttccgcctcccgtgatcgccgagaacagagccgagctgatgatcctgatcgccaccaacctgctggggcagaacacccccgccatcgccgtgaacgaggccgagtacggcgagatgtgggctcaagatgctgcagcgatgttcggatacgccgccgccacggcgaccgccaccgccaccttactgcccttcgaagaagcccccgagatgacaagcgccggcggcttattagagcaagccgcagccgtggaggaggctagcgacacggccgctgccaatcagctgatgaacaatgtgccccaagccctacaacaactggcgcagcccacccaaggcacaaccccaagcagcaagctgggcggcctgtggaaaaccgtgagcccccacagaagccccatcagcaacatggtgagcatggccaacaaccacatgagcatgacaaacagtggcgtgagcatgaccaatacgctcagcagcatgctgaagggcttcgcgccggccgccgctgcacaagctgttcagactgcggcgcagaacggggtgagagcaatgagcagcctgggctcaagccttggcagctctggcttaggcggcggtgtggcagccaacctgggcagagccgcctcagttgggagcctgtccgtgcctcaagcctgggcagcggcgaatcaagctgtcacccccgccgctagagccctgcccctgacaagcctgactagtgcggcagagagaggccccggccaaatgctcggtggcttaccggtagggcaaatgggggcaagagcgggaggcggtctttccggcgtgctgagagtgccccctagaccctacgtgatgccccacagccccgccgccggcggatctggcgagggcagaggcagcctgctgacctgcggcgacgtggaagagaaccccggccctgtggtggccgtgacctacagccccggcccccacctggagagattcctggctagcctgagcctggccaccgaaagacccgtgtccgtgcttttggccgacaacggcagtacggacggcaccccccaagccgccgtgcagagataccccaacgtgagactgctgcccaccggcgccaacctgggctacggcaccgccgtgaatagaacgatagctcagctgggcgagatggccggagacgccggtgaaccctgggtggacgattgggtcatagtggcgaatcccgacgtgcaatggggtcccggcagcatcgatgccctgctggatgccgctagcagatggccccgcgctggtgccctgggaccgttaattcgggaccccgacggcagcgtgtaccctagcgctagacagatgcctagcctgatcagaggcggcatgcacgccgtgctgggccccttctggcctagaaacccctggaccaccgcctacagacaagagagactggagcctagcgaaagacccgtgggctggctgtcgggctcctgcctgctggtgagaagaagcgccttcggccaagtgggcggcttcgacgagagatacttcatgtacatggaggacgtggacctgggcgacagactgggcaaggccggctggctaagcgtttacgtgcctagcgccgaggtgctgcaccacaaggcccacagcaccggccgggaccccgctagccacctggccgcccaccacaagagcacctacatcttcctggccgacagacacagcggctggtggagagcccccctgagatggaccctgagaggcagcctggccctgagaagccacctgatggtgagaagcagcctgagaagaagcagaagaagaaagctgaagctggtggagggcagacacggcggcggcggcggaggcgccgagatgaaaaccgacgccgccaccctggcccaagaggccggcaacttcgagagaatcagcggcgacctcaagacacagatcgaccaagtggagagcacggctggcagcctgcaagggcaatggagaggcgcagccggtactgccgcacaagccgccgtggtgagattccaagaggccgccaacaagcagaagcaagagctggacgagatcagcaccaacatcagacaagccggcgtgcagtacagcagagccgacgaggagcagcagcaagccctgagctctcagatgggcttctgataatgatag

SEQ ID NO: 32 - DNA, 12 bp, synthetic construct
  source 1..12: mol_type = other DNA
tgataatgatag

SEQ ID NO: 33 - protein, 912 aa, synthetic construct
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGGGVSFVMAYPEMLAAAADTLQSIGATTVASNAAAAAPTTGVVPPAADEVSALTAAHFAAHAAMYQSVSARAAAIHDQFVATLASSASSYAATEVANAAAASGGGGSGGGGSGGGGSVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGGGGGVVAVTYSPGPHLERFLASLSLATERPVSVLLADNGSTDGTPQAAVQRYPNVRLLPTGANLGYGTAVNRTIAQLGEMAGDAGEPWVDDWVIVANPDVQWGPGSIDALLDAASRWPRAGALGPLIRDPDGSVYPSARQMPSLIRGGMHAVLGPFWPRNPWTTAYRQERLEPSERPVGWLSGSCLLVRRSAFGQVGGFDERYFMYMEDVDLGDRLGKAGWLSVYVPSAEVLHHKAHSTGRDPASHLAAHHKSTYIFLADRHSGWWRAPLRWTLRGSLALRSHLMVRSSLRRSRRRKLKLVEGRH

SEQ ID NO: 34 - protein, 923 aa, synthetic construct
VVAVTYSPGPHLERFLASLSLATERPVSVLLADNGSTDGTPQAAVQRYPNVRLLPTGANLGYGTAVNRTIAQLGEMAGDAGEPWVDDWVIVANPDVQWGPGSIDALLDAASRWPRAGALGPLIRDPDGSVYPSARQMPSLIRGGMHAVLGPFWPRNPWTTAYRQERLEPSERPVGWLSGSCLLVRRSAFGQVGGFDERYFMYMEDVDLGDRLGKAGWLSVYVPSAEVLHHKAHSTGRDPASHLAAHHKSTYIFLADRHSGWWRAPLRWTLRGSLALRSHLMVRSSLRRSRRRKLKLVEGRHGGGGSGGGGSGGGGSAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGGGGGSGGGGSGGGGSVSFVMAYPEMLAAAADTLQSIGATTVASNAAAAAPTTGVVPPAADEVSALTAAHFAAHAAMYQSVSARAAAIHDQFVATLASSASSYAATEVANAAAAS

SEQ ID NO: 35 - protein, 912 aa, synthetic construct
VDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGGGGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGGGSGGGGSGGGGSVSFVMAYPEMLAAAADTLQSIGATTVASNAAAAAPTTGVVPPAADEVSALTAAHFAAHAAMYQSVSARAAAIHDQFVATLASSASSYAATEVANAAAASGGGGVVAVTYSPGPHLERFLASLSLATERPVSVLLADNGSTDGTPQAAVQRYPNVRLLPTGANLGYGTAVNRTIAQLGEMAGDAGEPWVDDWVIVANPDVQWGPGSIDALLDAASRWPRAGALGPLIRDPDGSVYPSARQMPSLIRGGMHAVLGPFWPRNPWTTAYRQERLEPSERPVGWLSGSCLLVRRSAFGQVGGFDERYFMYMEDVDLGDRLGKAGWLSVYVPSAEVLHHKAHSTGRDPASHLAAHHKSTYIFLADRHSGWWRAPLRWTLRGSLALRSHLMVRSSLRRSRRRKLKLVEGRH

SEQ ID NO: 36 - protein, 920 aa, synthetic construct
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGGGVVAVTYSPGPHLERFLASLSLATERPVSVLLADNGSTDGTPQAAVQRYPNVRLLPTGANLGYGTAVNRTIAQLGEMAGDAGEPWVDDWVIVANPDVQWGPGSIDALLDAASRWPRAGALGPLIRDPDGSVYPSARQMPSLIRGGMHAVLGPFWPRNPWTTAYRQERLEPSERPVGWLSGSCLLVRRSAFGQVGGFDERYFMYMEDVDLGDRLGKAGWLSVYVPSAEVLHHKAHSTGRDPASHLAAHHKSTYIFLADRHSGWWRAPLRWTLRGSLALRSHLMVRSSLRRSRRRKLKLVEGRHGSGATNFSLLKQAGDVEENPGPMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGGGGGVSFVMAYPEMLAAAADTLQSIGATTVASNAAAAAPTTGVVPPAADEVSALTAAHFAAHAAMYQSVSARAAAIHDQFVATLASSASSYAATEVANAAAAS

SEQ ID NO: 37 - DNA, 2736 bp, synthetic construct
  source 1..2736: mol_type = other DNA
gccgagatgaaaaccgacgccgccaccctggcccaagaggccggcaacttcgagagaatcagcggcgacctcaagacacagatcgaccaagtggagagcacggctggcagcctgcaagggcaatggagaggcgcagccggtactgccgcacaagccgccgtggtgagattccaagaggccgccaacaagcagaagcaagagctggacgagatcagcaccaacatcagacaagccggcgtgcagtacagcagagccgacgaggagcagcagcaagccctgagctctcagatgggcttcggcggcggcggcgtgtctttcgtgatggcctaccccgaaatgctagccgccgccgcagataccctgcagagcatcggcgcaaccacagttgcaagcaatgctgctgctgccgcaccgactaccggcgtggtgccccccgcggcggatgaggtgagcgccctaaccgccgcccatttcgccgcacacgccgcgatgtatcagagcgtgagcgctagagccgccgccatccacgatcagttcgtggccaccctggctagcagcgctagcagctacgccgccaccgaggtggccaacgcggccgccgctagcggcggaggcggatctggcggaggcggcagcggcggcggcggcagcgtggacttcggcgccctgccccccgagataaactccgctagaatgtacgccggcccgggaagcgctagcttggtcgcagccgcacagatgtgggacagcgtggctagcgacctgttcagcgccgctagcgcctttcagtcggtggtgtggggacttactgtcggcagctggatcggcagttcggctggcttgatggtggccgcggcgtcgccctatgtggcctggatgagcgtgactgccgggcaagccgaactgactgcggctcaagtgcgtgtggccgcggctgcctatgaaaccgcgtacggccttacagttccgcctcccgtgatcgccgagaacagagccgagctgatgatcctgatcgccaccaacctgctggggcagaacacccccgccatcgccgtgaacgaggccgagtacggcgagatgtgggctcaagatgctgcagcgatgttcggatacgccgccgccacggcgaccgccaccgccaccttactgcccttcgaagaagcccccgagatgacaagcgccggcggcttattagagcaagccgcagccgtggaggaggctagcgacacggccgctgccaatcagctgatgaacaatgtgccccaagccctacaacaactggcgcagcccacccaaggcacaaccccaagcagcaagctgggcggcctgtggaaaaccgtgagcccccacagaagccccatcagcaacatggtgagcatggccaacaaccacatgagcatgacaaacagtggcgtgagcatgaccaatacgctcagcagcatgctgaagggcttcgcgccggccgccgctgcacaagctgttcagactgcggcgcagaacggggtgagagcaatgagcagcctgggctcaagccttggcagctctggcttaggcggcggtgtggcagccaacctgggcagagccgcctcagttgggagcctgtccgtgcctcaagcctgggcagcggcgaatcaagctgtcacccccgccgctagagccctgcccctgacaagcctgactagtgcggcagagagaggccccggccaaatgctcggtggcttaccggtagggcaaatgggggcaagagcgggaggcggtctttccggcgtgctgagagtgccccctagaccctacgtgatgccccacagccccgccgccggcggcggcggcggcgtggtggccgtgacctacagccccggcccccacctggagagattcctggctagcctgagcctggccaccgaaagacccgtgtccgtgcttttggccgacaacggcagtacggacggcaccccccaagccgccgtgcagagataccccaacgtgagactgctgcccaccggcgccaacctgggctacggcaccgccgtgaatagaacgatagctcagctgggcgagatggccggagacgccggtgaaccctgggtggacgattgggtcatagtggcgaatcccgacgtgcaatggggtcccggcagcatcgatgccctgctggatgccgctagcagatggccccgcgctggtgccctgggaccgttaattcgggaccccgacggcagcgtgtaccctagcgctagacagatgcctagcctgatcagaggcggcatgcacgccgtgctgggccccttctggcctagaaacccctggaccaccgcctacagacaagagagactggagcctagcgaaagacccgtgggctggctgtcgggctcctgcctgctggtgagaagaagcgccttcggccaagtgggcggcttcgacgagagatacttcatgtacatggaggacgtggacctgggcgacagactgggcaaggccggctggctaagcgtttacgtgcctagcgccgaggtgctgcaccacaaggcccacagcaccggccgggaccccgctagccacctggccgcccaccacaagagcacctacatcttcctggccgacagacacagcggctggtggagagcccccctgagatggaccctgagaggcagcctggccctgagaagccacctgatggtgagaagcagcctgagaagaagcagaagaagaaagctgaagctggtggagggcagacac

SEQ ID NO: 38 - DNA, 2769 bp, synthetic construct
  source 1..2769: mol_type = other DNA
gtggtggccgtgacctacagccccggcccccacctggagagattcctggctagcctgagcctggccaccgaaagacccgtgtccgtgcttttggccgacaacggcagtacggacggcaccccccaagccgccgtgcagagataccccaacgtgagactgctgcccaccggcgccaacctgggctacggcaccgccgtgaatagaacgatagctcagctgggcgagatggccggagacgccggtgaaccctgggtggacgattgggtcatagtggcgaatcccgacgtgcaatggggtcccggcagcatcgatgccctgctggatgccgctagcagatggccccgcgctggtgccctgggaccgttaattcgggaccccgacggcagcgtgtaccctagcgctagacagatgcctagcctgatcagaggcggcatgcacgccgtgctgggccccttctggcctagaaacccctggaccaccgcctacagacaagagagactggagcctagcgaaagacccgtgggctggctgtcgggctcctgcctgctggtgagaagaagcgccttcggccaagtgggcggcttcgacgagagatacttcatgtacatggaggacgtggacctgggcgacagactgggcaaggccggctggctaagcgtttacgtgcctagcgccgaggtgctgcaccacaaggcccacagcaccggccgggaccccgctagccacctggccgcccaccacaagagcacctacatcttcctggccgacagacacagcggctggtggagagcccccctgagatggaccctgagaggcagcctggccctgagaagccacctgatggtgagaagcagcctgagaagaagcagaagaagaaagctgaagctggtggagggcagacacggcggaggcggatctggcggaggcggcagcggcggcggcggcagcgccgagatgaaaaccgacgccgccaccctggcccaagaggccggcaacttcgagagaatcagcggcgacctcaagacacagatcgaccaagtggagagcacggctggcagcctgcaagggcaatggagaggcgcagccggtactgccgcacaagccgccgtggtgagattccaagaggccgccaacaagcagaagcaagagctggacgagatcagcaccaacatcagacaagccggcgtgcagtacagcagagccgacgaggagcagcagcaagccctgagctctcagatgggcttcggcggcggcggcgtggacttcggcgccctgccccccgagataaactccgctagaatgtacgccggcccgggaagcgctagcttggtcgcagccgcacagatgtgggacagcgtggctagcgacctgttcagcgccgctagcgcctttcagtcggtggtgtggggacttactgtcggcagctggatcggcagttcggctggcttgatggtggccgcggcgtcgccctatgtggcctggatgagcgtgactgccgggcaagccgaactgactgcggctcaagtgcgtgtggccgcggctgcctatgaaaccgcgtacggccttacagttccgcctcccgtgatcgccgagaacagagccgagctgatgatcctgatcgccaccaacctgctggggcagaacacccccgccatcgccgtgaacgaggccgagtacggcgagatgtgggctcaagatgctgcagcgatgttcggatacgccgccgccacggcgaccgccaccgccaccttactgcccttcgaagaagcccccgagatgacaagcgccggcggcttattagagcaagccgcagccgtggaggaggctagcgacacggccgctgccaatcagctgatgaacaatgtgccccaagccctacaacaactggcgcagcccacccaaggcacaaccccaagcagcaagctgggcggcctgtggaaaaccgtgagcccccacagaagccccatcagcaacatggtgagcatggccaacaaccacatgagcatgacaaacagtggcgtgagcatgaccaatacgctcagcagcatgctgaagggcttcgcgccggccgccgctgcacaagctgttcagactgcggcgcagaacggggtgagagcaatgagcagcctgggctcaagccttggcagctctggcttaggcggcggtgtggcagccaacctgggcagagccgcctcagttgggagcctgtccgtgcctcaagcctgggcagcggcgaatcaagctgtcacccccgccgctagagccctgcccctgacaagcctgactagtgcggcagagagaggccccggccaaatgctcggtggcttaccggtagggcaaatgggggcaagagcgggaggcggtctttccggcgtgctgagagtgccccctagaccctacgtgatgccccacagccccgccgccggcggcggaggcggatctggcggaggcggcagcggcggcggcggcagcgtgtctttcgtgatggcctaccccgaaatgctagccgccgccgcagataccctgcagagcatcggcgcaaccacagttgcaagcaatgctgctgctgccgcaccgactaccggcgtggtgccccccgcggcggatgaggtgagcgccctaaccgccgcccatttcgccgcacacgccgcgatgtatcagagcgtgagcgctagagccgccgccatccacgatcagttcgtggccaccctggctagcagcgctagcagctacgccgccaccgaggtggccaacgcggccgccgctagc

SEQ ID NO: 39 - DNA, 2736 bp, synthetic construct
  source 1..2736: mol_type = other DNA
gtggacttcggcgccctgccccccgagataaactccgctagaatgtacgccggcccgggaagcgctagcttggtcgcagccgcacagatgtgggacagcgtggctagcgacctgttcagcgccgctagcgcctttcagtcggtggtgtggggacttactgtcggcagctggatcggcagttcggctggcttgatggtggccgcggcgtcgccctatgtggcctggatgagcgtgactgccgggcaagccgaactgactgcggctcaagtgcgtgtggccgcggctgcctatgaaaccgcgtacggccttacagttccgcctcccgtgatcgccgagaacagagccgagctgatgatcctgatcgccaccaacctgctggggcagaacacccccgccatcgccgtgaacgaggccgagtacggcgagatgtgggctcaagatgctgcagcgatgttcggatacgccgccgccacggcgaccgccaccgccaccttactgcccttcgaagaagcccccgagatgacaagcgccggcggcttattagagcaagccgcagccgtggaggaggctagcgacacggccgctgccaatcagctgatgaacaatgtgccccaagccctacaacaactggcgcagcccacccaaggcacaaccccaagcagcaagctgggcggcctgtggaaaaccgtgagcccccacagaagccccatcagcaacatggtgagcatggccaacaaccacatgagcatgacaaacagtggcgtgagcatgaccaatacgctcagcagcatgctgaagggcttcgcgccggccgccgctgcacaagctgttcagactgcggcgcagaacggggtgagagcaatgagcagcctgggctcaagccttggcagctctggcttaggcggcggtgtggcagccaacctgggcagagccgcctcagttgggagcctgtccgtgcctcaagcctgggcagcggcgaatcaagctgtcacccccgccgctagagccctgcccctgacaagcctgactagtgcggcagagagaggccccggccaaatgctcggtggcttaccggtagggcaaatgggggcaagagcgggaggcggtctttccggcgtgctgagagtgccccctagaccctacgtgatgccccacagccccgccgccggcggcggcggcggcgccgagatgaaaaccgacgccgccaccctggcccaagaggccggcaacttcgagagaatcagcggcgacctcaagacacagatcgaccaagtggagagcacggctggcagcctgcaagggcaatggagaggcgcagccggtactgccgcacaagccgccgtggtgagattccaagaggccgccaacaagcagaagcaagagctggacgagatcagcaccaacatcagacaagccggcgtgcagtacagcagagccgacgaggagcagcagcaagccctgagctctcagatgggcttcggcggaggcggatctggcggaggcggcagcggcggcggcggcagcgtgtctttcgtgatggcctaccccgaaatgctagccgccgccgcagataccctgcagagcatcggcgcaaccacagttgcaagcaatgctgctgctgccgcaccgactaccggcgtggtgccccccgcggcggatgaggtgagcgccctaaccgccgcccatttcgccgcacacgccgcgatgtatcagagcgtgagcgctagagccgccgccatccacgatcagttcgtggccaccctggctagcagcgctagcagctacgccgccaccgaggtggccaacgcggccgccgctagcggcggcggcggcgtggtggccgtgacctacagccccggcccccacctggagagattcctggctagcctgagcctggccaccgaaagacccgtgtccgtgcttttggccgacaacggcagtacggacggcaccccccaagccgccgtgcagagataccccaacgtgagactgctgcccaccggcgccaacctgggctacggcaccgccgtgaatagaacgatagctcagctgggcgagatggccggagacgccggtgaaccctgggtggacgattgggtcatagtggcgaatcccgacgtgcaatggggtcccggcagcatcgatgccctgctggatgccgctagcagatggccccgcgctggtgccctgggaccgttaattcgggaccccgacggcagcgtgtaccctagcgctagacagatgcctagcctgatcagaggcggcatgcacgccgtgctgggccccttctggcctagaaacccctggaccaccgcctacagacaagagagactggagcctagcgaaagacccgtgggctggctgtcgggctcctgcctgctggtgagaagaagcgccttcggccaagtgggcggcttcgacgagagatacttcatgtacatggaggacgtggacctgggcgacagactgggcaaggccggctggctaagcgtttacgtgcctagcgccgaggtgctgcaccacaaggcccacagcaccggccgggaccccgctagccacctggccgcccaccacaagagcacctacatcttcctggccgacagacacagcggctggtggagagcccccctgagatggaccctgagaggcagcctggccctgagaagccacctgatggtgagaagcagcctgagaagaagcagaagaagaaagctgaagctggtggagggcagacac

SEQ ID NO: 40 - DNA, 2760 bp, synthetic construct
  source 1..2760: mol_type = other DNA
gccgagatgaaaaccgacgccgccaccctggcccaagaggccggcaacttcgagagaatcagcggcgacctcaagacacagatcgaccaagtggagagcacggctggcagcctgcaagggcaatggagaggcgcagccggtactgccgcacaagccgccgtggtgagattccaagaggccgccaacaagcagaagcaagagctggacgagatcagcaccaacatcagacaagccggcgtgcagtacagcagagccgacgaggagcagcagcaagccctgagctctcagatgggcttcggcggcggcggcgtggtggccgtgacctacagccccggcccccacctggagagattcctggctagcctgagcctggccaccgaaagacccgtgtccgtgcttttggccgacaacggcagtacggacggcaccccccaagccgccgtgcagagataccccaacgtgagactgctgcccaccggcgccaacctgggctacggcaccgccgtgaatagaacgatagctcagctgggcgagatggccggagacgccggtgaaccctgggtggacgattgggtcatagtggcgaatcccgacgtgcaatggggtcccggcagcatcgatgccctgctggatgccgctagcagatggccccgcgctggtgccctgggaccgttaattcgggaccccgacggcagcgtgtaccctagcgctagacagatgcctagcctgatcagaggcggcatgcacgccgtgctgggccccttctggcctagaaacccctggaccaccgcctacagacaagagagactggagcctagcgaaagacccgtgggctggctgtcgggctcctgcctgctggtgagaagaagcgccttcggccaagtgggcggcttcgacgagagatacttcatgtacatggaggacgtggacctgggcgacagactgggcaaggccggctggctaagcgtttacgtgcctagcgccgaggtgctgcaccacaaggcccacagcaccggccgggaccccgctagccacctggccgcccaccacaagagcacctacatcttcctggccgacagacacagcggctggtggagagcccccctgagatggaccctgagaggcagcctggccctgagaagccacctgatggtgagaagcagcctgagaagaagcagaagaagaaagctgaagctggtggagggcagacacggaagcggagctactaacttcagcctgctgaagcaggctggggacgtggaggagaaccctggacctatggtggacttcggcgccctgccccccgagataaactccgctagaatgtacgccggcccgggaagcgctagcttggtcgcagccgcacagatgtgggacagcgtggctagcgacctgttcagcgccgctagcgcctttcagtcggtggtgtggggacttactgtcggcagctggatcggcagttcggctggcttgatggtggccgcggcgtcgccctatgtggcctggatgagcgtgactgccgggcaagccgaactgactgcggctcaagtgcgtgtggccgcggctgcctatgaaaccgcgtacggccttacagttccgcctcccgtgatcgccgagaacagagccgagctgatgatcctgatcgccaccaacctgctggggcagaacacccccgccatcgccgtgaacgaggccgagtacggcgagatgtgggctcaagatgctgcagcgatgttcggatacgccgccgccacggcgaccgccaccgccaccttactgcccttcgaagaagcccccgagatgacaagcgccggcggcttattagagcaagccgcagccgtggaggaggctagcgacacggccgctgccaatcagctgatgaacaatgtgccccaagccctacaacaactggcgcagcccacccaaggcacaaccccaagcagcaagctgggcggcctgtggaaaaccgtgagcccccacagaagccccatcagcaacatggtgagcatggccaacaaccacatgagcatgacaaacagtggcgtgagcatgaccaatacgctcagcagcatgctgaagggcttcgcgccggccgccgctgcacaagctgttcagactgcggcgcagaacggggtgagagcaatgagcagcctgggctcaagccttggcagctctggcttaggcggcggtgtggcagccaacctgggcagagccgcctcagttgggagcctgtccgtgcctcaagcctgggcagcggcgaatcaagctgtcacccccgccgctagagccctgcccctgacaagcctgactagtgcggcagagagaggccccggccaaatgctcggtggcttaccggtagggcaaatgggggcaagagcgggaggcggtctttccggcgtgctgagagtgccccctagaccctacgtgatgccccacagccccgccgccggcggcggcggcggcgtgtctttcgtgatggcctaccccgaaatgctagccgccgccgcagataccctgcagagcatcggcgcaaccacagttgcaagcaatgctgctgctgccgcaccgactaccggcgtggtgccccccgcggcggatgaggtgagcgccctaaccgccgcccatttcgccgcacacgccgcgatgtatcagagcgtgagcgctagagccgccgccatccacgatcagttcgtggccaccctggctagcagcgctagcagctacgccgccaccgaggtggccaacgcggccgccgctagc

(19) United States
(12) Patent Application Publication
(10) Pub. No.: US 2026/0041750 A1
(43) Pub. Date: Feb. 12, 2026

(54) VACCINE CONSTRUCTS COMPRISING TUBERCULOSIS ANTIGENS

(71) Applicants: UNIVERSITY OF CAPE TOWN, Cape Town (ZA); UNIVERSITY OF THE WITWATERSRAND, JOHANNESBURG, Johannesburg (ZA); THE BOARD OF TRUSTEES OF THE LELAND STANFORD JUNIOR UNIVERSITY, Stanford, CA (US)

(72) Inventors: Munyaradzi N. MUSVOSVI, Cape Town (ZA); Thomas J. SCRIBA, Cape Town (ZA); Abdullah ELY, Johannesburg (ZA); Kristie BLOOM, Johannesburg (ZA); Patrick ARBUTHNOT, Johannesburg (ZA); Gerlinde OBERMOSER, Stanford, CA (US); Huang HUANG, Stanford, CA (US); Mark M. DAVIS, Stanford, CA (US)

(21) Appl. No.: 19/099,567

(22) PCT Filed: Jul. 28, 2023
(86) PCT No.: PCT/IB2023/057700

§ 371 Date: Jan. 29, 2025

(30) Foreign Application Priority Data
Jul. 29, 2022 (GB) 2211137.1
Jun. 1, 2023 (ZA) 2023/05862

(51) Int. Cl. A61K 39/04; A61K 9/1272; A61K 9/51; A61K 39/00; A61P 31/06; C07K 14/35

(52) U.S. Cl. CPC A61K 39/04; A61K 9/1272; A61K 9/5123; A61P 31/06; C07K 14/35; A61K 2039/53; A61K 2039/55555; C07K 2319/02

(57) ABSTRACT

The present invention relates to polygenic nucleic acid constructs comprising nucleotide sequences encoding Mycobacterium tuberculosis antigens and to mRNA vaccine constructs transcribed or obtained therefrom. Also provided are lipid nanoparticles including the mRNA vaccine constructs and vaccine compositions comprising the constructs described. The constructs, lipid nanoparticles containing them, and vaccine compositions described may be useful in methods for eliciting a protective immune response against Mycobacterium tuberculosis in a subject.

### RELATED APPLICATIONS

This application is a National Stage Application of International Application No. PCT/IB2023/057700, filed Jul. 28, 2023, which claims priority to GB Patent Application No. 2211137.1, filed Jul. 29, 2022, and ZA Patent Application No. 2023/05862, filed Jun. 1, 2023, the disclosures of which are incorporated by reference herein in their entirety.

### REFERENCE TO THE SEQUENCE LISTING

The Sequence Listing associated with this application is provided in XML format. The XML file containing the Sequence Listing is entitled “P4166US00_Sequence_Listing.XML”, has a size of 60 KB, and was created on Jul. 27, 2023. The contents of the XML file are incorporated by reference in their entirety.

### BACKGROUND OF THE INVENTION

The invention relates to the identification of Mycobacterium tuberculosis (M.tb) antigens recognized by antigen-specific T cells associated with control of tuberculosis (TB) disease. The invention further relates to the use of antigens identified as being the target of these antigen-specific T cells in the development of an mRNA vaccine construct that induces an immune response to TB. The present invention also relates to vaccine compositions comprising at least one mRNA encoding at least one or more of the identified antigens for use in eliciting an immune response against TB.

TB is a disease that causes major morbidity and mortality around the globe, which disproportionally affects middle and low-income countries. Until 2019, and since the end of the Covid-19 pandemic, TB has been and is again the biggest infectious killer of humans from a single pathogen. TB is also a major burden to healthcare systems globally and an efficacious vaccine would help society by reducing disease burden.

Antigen-specific CD4 T cells are necessary for protective immunity against M.tb, the etiological agent of TB, but the ability to broadly study these responses has been limited. Experimental and clinical evidence show that the primary T cell mediators of this protection are IFN-γ-expressing Th1 cells, although recent evidence from non-human primates implicates Th1/Th17 cells as likely correlates of protection. Comprehensive delineation of αβ T cell responses in M.tb-infected humans has been hampered by the complexity and heterogeneity of clinical phenotypes in TB, the high inter-individual diversity of major histocompatibility complex (MHC), which restricts antigen presentation to T cells, and the marked diversity of T cell receptors (TCRs), even within single hosts.

Various TB vaccine candidates which contain M.tb proteins and that aim to protect against TB exist. Some of these candidate vaccines exist as subunit vaccines, which contain certain M.tb proteins, other candidate vaccines are live attenuated M.tb, while others are genetically modified versions of the Bacillus Calmette-Guérin (BCG) vaccine.

The inventors of the present invention have developed a monogenic and polygenic vaccine constructs comprising either one or four antigens for inducing an immunological response against M.tb.

### SUMMARY OF THE INVENTION

The present invention relates to monogenic and polygenic nucleic acid constructs comprising nucleotide sequences encoding Mycobacterium tuberculosis (M.tb) antigens and to mRNA vaccine constructs transcribed or obtained therefrom. The invention further relates to lipid nanoparticles including the mRNA vaccine constructs and to vaccine compositions comprising the constructs described. The constructs, lipid nanoparticles containing them, and vaccine compositions described may be useful in methods for eliciting an immune response against M.tb in a subject.

According to a first aspect of the present invention there is provided for a monogenic nucleic acid construct comprising at least one nucleotide sequence selected from the group consisting of: a) a nucleotide sequence encoding a WbbL1 antigen having the amino acid sequence of SEQ ID NO:1; b) a nucleotide sequence encoding a CFP-10 antigen having the amino acid sequence of SEQ ID NO:2; c) a nucleotide sequence encoding a PPE18 antigen having the amino acid sequence of SEQ ID NO:3; and d) a nucleotide sequence encoding a PE13 antigen having the amino acid sequence of SEQ ID NO: 4; wherein two or more of the monogenic nucleic acid constructs may be provided to a subject in a combination; and/or a polygenic nucleic acid construct comprising at least two nucleotide sequences selected from the group consisting of: a) a nucleotide sequence encoding a WbbL1 antigen having the amino acid sequence of SEQ ID NO:1; b) a nucleotide sequence encoding a CFP-10 antigen having the amino acid sequence of SEQ ID NO: 2; c) a nucleotide sequence encoding a PPE18 antigen having the amino acid sequence of SEQ ID NO:3; and d) a nucleotide sequence encoding a PE13 antigen having the amino acid sequence of SEQ ID NO:4.

According to a first embodiment of the monogenic and polygenic nucleic acid construct of the invention, (a) the nucleotide sequence encoding a WbbL1 antigen has a nucleic acid sequence substantially identical to SEQ ID NO:5, (b) the nucleotide sequence encoding a CFP-10 antigen has a nucleic acid sequence substantially identical to SEQ ID NO: 6, (c) the nucleotide sequence encoding a PPE18 antigen has a nucleic acid sequence substantially identical to SEQ ID NO:7, and (d) the nucleotide sequence encoding a PE13 antigen has a nucleic acid sequence substantially identical to SEQ ID NO:8.

In a second embodiment of the polygenic nucleic acid construct of the invention, each of the at least two nucleotide sequences may be separated by a nucleotide sequence encoding a linker. Several linkers are known in the art, and the linker may be selected from the group consisting of a glycine-serine flexible linker, a glycine flexible linker, and a 2A-derived peptide. In particular, each linker may independently have a sequence selected from the group consisting of the linker sequences set forth in any one of SEQ ID NOs: 9-19.

According to a third embodiment of the monogenic and polygenic nucleic acid construct of the invention, the nucleic acid construct may further comprise a leader nucleotide sequence encoding a secretory peptide signal. In some embodiments of the polygenic nucleic acid construct of the invention, each of the at least two nucleotide sequences comprise a leader nucleotide sequence encoding a secretory peptide signal. The leader sequence may be derived from a M.tb secretory peptide signal or from another source. In particular, each secretory peptide signal may independently have an amino acid sequence selected from the group consisting of SEQ ID NO:20, SEQ ID NO:22, SEQ ID NO: 24, SEQ ID NO:26, and SEQ ID NO:28. Further, the nucleotide sequence encoding each secretory peptide signal may be encoded by a nucleotide sequence substantially identical to a sequence set forth in the group consisting of SEQ ID NO:21, SEQ ID NO:23, SEQ ID NO:25, SEQ ID NO:27, and SEQ ID NO:29. It will be appreciated by those of skill in the art that the leader sequence is usually positioned upstream, preferably in-frame, of the antigenic polypeptide(s). However, in some suitable embodiments of the invention, the leader may also be included downstream of the antigenic polypeptide(s).

In a fourth embodiment of the polygenic nucleic acid construct of the invention, the polygenic nucleic acid construct may comprise all of the nucleotide sequences in (a) to (d). Exemplary polygenic nucleic acid constructs of the invention include a polygenic nucleic acid construct encoding a polyprotein having an amino acid sequence of any one of SEQ ID NO: 33-36. For example, the polygenic nucleic acid construct may have a nucleic acid sequence substantially identical to any one of SEQ ID NOs: 37-40.

It will be further be appreciated by those of skill in the art, that the first (N-terminal) antigenic sequence in the mono/polygenic construct will include a start codon (ATG). However, when the mono/polygenic construct comprises a leader sequence, the first antigenic sequence would not include the first codon (start codon) as the leader sequence includes the start codon (ATG). Individual antigenic sequences may also include a start codon. Similarly, the last (C-terminal) antigenic sequence in the construct may include one or more stop codons, including two, three or four stop codons, each having the sequence TAA, TAG, or TGA. In particular, the last sequence includes a stop codon having a design consisting of four consecutive stop sequences in a row, with the nucleotide sequence of TGATAATGATAG (SEQ ID NO:32). Individual antigenic sequences may also include the stop sequence of SED ID NO: 32.

According to a second aspect of the present invention there is provided for an mRNA construct transcribed from the mono/polygenic nucleic acid construct described herein.

In a first embodiment of the mRNA construct of the invention, the mRNA construct may be capped at the 5′ end.

According to a second embodiment of the mRNA construct of the invention, the mRNA construct may include one or more modified nucleotides. For example, the mRNA construct may include one or more modified nucleotides selected from the group consisting of N1-methyl-pseudouridine and pseudouridine.

According to a third aspect of the present invention, there is provided for a lipid nanoparticle comprising the mRNA constructs described herein.

According to a fourth aspect of the present invention there is provided for a vaccine composition comprising the mono/polygenic nucleic acid construct described herein, or the mRNA construct described herein, or the lipid nanoparticle described herein, and one or more pharmaceutically acceptable diluents or excipients, wherein the vaccine composition is capable of eliciting an immune response against M.tb. In some embodiments, a combination of two or more of the monogenic nucleic acid constructs or mRNA constructs are provided in a single vaccine composition.

In one aspect of the present invention there is provided for a vaccine composition comprising at least two nucleic acids selected from: a) a nucleic acid encoding a WbbL antigen having the amino acid sequence of SEQ ID NO:1; b) a nucleic acid encoding a CFP-10 antigen having the amino acid sequence of SEQ ID NO:2; c) a nucleic acid encoding a PPE18 antigen having the amino acid sequence of SEQ ID NO:3; and d) a nucleic acid encoding a PE13 antigen having the amino acid sequence of SEQ ID NO:4.

In a first embodiment of the vaccine composition, the vaccine composition may comprise all of the nucleic acids in (a) to (d).

According to a second embodiment of the vaccine composition, (a) has a nucleotide sequence substantially identical to SEQ ID NO:5, (b) has a nucleotide sequence substantially identical to SEQ ID NO:6, (c) has a nucleotide sequence substantially identical to SEQ ID NO:7, and (d) has a nucleotide sequence substantially identical to SEQ ID NO:8.

In a third embodiment of the vaccine composition, at least one of the nucleic acids may further comprise a leader nucleotide sequence encoding a secretory peptide signal. In some embodiments, each of the nucleic acids may comprise a leader nucleotide sequence encoding a secretory peptide signal. The leader sequence may be derived from a M.tb secretory peptide signal or from another source. In particular, each secretory peptide signal may independently have an amino acid sequence selected from the group consisting of SEQ ID NO: 20, SEQ ID NO:22, SEQ ID NO: 24, SEQ ID NO:26, and SEQ ID NO:28. Further, the nucleotide sequence encoding each secretory peptide signal may be encoded by a nucleotide sequence substantially identical to a sequence set forth in the group consisting of SEQ ID NO:21, SEQ ID NO:23, SEQ ID NO:25, SEQ ID NO:27, and SEQ ID NO:29. It will be appreciated by those of skill in the art that the leader sequence is usually positioned upstream, preferably in-frame, of the sequence encoding the antigenic polypeptide(s). However, in some suitable embodiments of the invention, the leader may also be included downstream of the antigenic polypeptide(s).

According to a fourth embodiment of the vaccine composition of the invention, the at least two nucleic acid sequences may be provided on a single nucleic acid construct.

In a further embodiment of the vaccine composition, each of the nucleic acids provided on the nucleic acid construct may be separated by a nucleotide sequence encoding a linker. Several linkers are known in the art, and the linker may be selected from the group consisting of a glycine-serine flexible linker, a glycine flexible linker, and a 2A-derived peptide. In particular, each linker may independently have a sequence selected from the group consisting of the linker sequences set forth in any one of SEQ ID NOs: 9-19.

In yet a further embodiment of the vaccine composition of the invention, the nucleic acid construct may encode a polyprotein having an amino acid sequence of any one of SEQ ID NO: 33-36. For example, the polygenic nucleic acid construct may have a nucleic acid sequence substantially identical to any one of SEQ ID NOs: 37-40. It will be further be appreciated by those of skill in the art, that the first (N-terminal) antigenic sequence in the polygenic nucleic acid construct will include a start codon (ATG). However, when the polygenic construct comprises a leader sequence, the first antigenic sequence would not include the first codon (start codon) as the leader sequence includes the start codon (ATG). Similarly, the last (C-terminal) antigenic sequence in the polygenic nucleic acid construct may include one or more stop codons, including two, three or four stop codons, each having the sequence TAA, TAG, or TGA. In particular, the last sequence includes a stop codon having a design consisting of four consecutive stop sequences in a row, with the nucleotide sequence of TGATAATGATAG (SEQ ID NO:32).

In another embodiment of the vaccine composition, the nucleic acid may be mRNA or the nucleic acid construct may be an mRNA construct.

In some embodiments of the vaccine composition of the invention, the mRNA or mRNA construct may be capped at the 5′ end.

According to a further embodiment of the vaccine composition of the invention, the mRNA or mRNA construct may include one or more modified nucleotides. For example, the mRNA or mRNA construct may include one or more modified nucleotides selected from the group consisting of N1-methyl-pseudouridine and pseudouridine.

In another embodiment of the vaccine composition, the mRNA may be comprised in a lipid nanoparticle.

In a further embodiment of the vaccine composition, the vaccine composition is capable of eliciting a protective immune response against Mycobacterium tuberculosis.

According to a further aspect there is provided for an mRNA vaccine composition comprising at least two mRNAs selected from: a) an mRMA encoding a WbbL antigen having the amino acid sequence of SEQ ID NO:1; b) an mRNA encoding a CFP-10 antigen having the amino acid sequence of SEQ ID NO:2; c) an mRNA encoding a PPE18 antigen having the amino acid sequence of SEQ ID NO:3; and d) an mRNA encoding a PE13 antigen having the amino acid sequence of SEQ ID NO:4.

In a first embodiment of the mRNA vaccine composition, the mRNA vaccine composition may comprise all of the mRNAs in (a) to (d).

According to a second embodiment of the mRNA vaccine composition, (a) is transcribed from a nucleotide sequence substantially identical to SEQ ID NO:5, (b) is transcribed from a nucleotide sequence substantially identical to SEQ ID NO:6, (c) is transcribed from a nucleotide sequence substantially identical to SEQ ID NO:7, and (d) is transcribed from a nucleotide sequence substantially identical to SEQ ID NO:8.

In a third embodiment of the mRNA vaccine composition, a nucleic acid transcribing at least one of the mRNAs may further comprise a leader nucleotide sequence encoding a secretory peptide signal. In some embodiments, each of the nucleic acids may comprise a leader nucleotide sequence encoding a secretory peptide signal. The leader sequence may be derived from a M.tb secretory peptide signal or from another source. In particular, each secretory peptide signal may independently have an amino acid sequence selected from the group consisting of SEQ ID NO:20, SEQ ID NO:22, SEQ ID NO:24, SEQ ID NO: 26, and SEQ ID NO:28. Further, the nucleotide sequence encoding each secretory peptide signal may be encoded by a nucleotide sequence substantially identical to a sequence set forth in the group consisting of SEQ ID NO:21, SEQ ID NO:23, SEQ ID NO:25, SEQ ID NO:27, and SEQ ID NO:29. It will be appreciated by those of skill in the art that the leader sequence is usually positioned upstream, preferably in-frame, of the sequence encoding the antigenic polypeptide(s). However, in some suitable embodiments of the invention, the leader may also be included downstream of the antigenic polypeptide(s).

According to a fourth embodiment of the mRNA vaccine composition of the invention, the at least two mRNAs may be transcribed from a single nucleic acid construct.

In a further embodiment of the mRNA vaccine composition, each of the nucleic acids provided on the nucleic acid construct may be separated by a nucleotide sequence encoding a linker. Several linkers are known in the art, and the linker may be selected from the group consisting of a glycine-serine flexible linker, a glycine flexible linker, and a 2A-derived peptide. In particular, each linker may independently have a sequence selected from the group consisting of the linker sequences set forth in any one of SEQ ID NOs: 9-19.

In yet a further embodiment of the mRNA vaccine composition of the invention, the mRNA may be transcribed from a nucleic acid construct encoding a polyprotein having an amino acid sequence of any one of SEQ ID NO:33-36. For example, the mRNA may be transcribed from a polygenic nucleic acid construct having a nucleic acid sequence substantially identical to any one of SEQ ID NOs: 37-40. It will be further be appreciated by those of skill in the art, that the first (N-terminal) antigenic sequence in the polygenic nucleic acid construct will include a start codon (ATG). However, when the polygenic construct comprises a leader sequence, the first antigenic sequence would not include the first codon (start codon) as the leader sequence includes the start codon (ATG). Similarly, the last (C-terminal) antigenic sequence in the polygenic nucleic acid construct may include one or more stop codons, including two, three or four stop codons, each having the sequence TAA, TAG, or TGA. In particular, the last sequence includes a stop codon having a design consisting of four consecutive stop sequences in a row, with the nucleotide sequence of TGATAATGATAG (SEQ ID NO:32).

According to one embodiment of the mRNA vaccine composition of the invention, the mRNA may be capped at the 5′ end.

According to a further embodiment of the mRNA vaccine composition of the invention, the mRNA may include one or more modified nucleotides. For example, the mRNA may include one or more modified nucleotides selected from the group consisting of N1-methyl-pseudouridine and pseudouridine.

In a further embodiment of the mRNA vaccine composition, the mRNA may be comprised in a lipid nanoparticle.

In a further embodiment of the mRNA vaccine composition, the vaccine composition is capable of eliciting a protective immune response against Mycobacterium tuberculosis.

In a further aspect of the present invention there is provided for a monogenic and/or polygenic nucleic acid construct, an mRNA construct, a lipid nanoparticle, a vaccine composition, or an mRNA vaccine composition as described herein for use in a method of eliciting an immune response against M.tb, wherein the method comprises administering the vaccine composition to a subject.

Further provided for in another aspect of the invention is a method of eliciting an immune response against M.tb in a subject, the method comprising administering the monogenic and/or polygenic nucleic acid construct, the mRNA construct, the lipid nanoparticle, the vaccine composition, or the mRNA vaccine composition as described herein to the subject.

According to yet another aspect of the present invention there is provided for the use of the mono/polygenic nucleic acid construct, the mRNA construct, the lipid nanoparticle, the vaccine composition or the mRNA vaccine composition described herein in the manufacture of a vaccine for use in a method of eliciting an immune response against M.tb, the method comprising administering the vaccine to a subject.

### BRIEF DESCRIPTION OF THE FIGURES

Non-limiting embodiments of the invention will now be described by way of example only and with reference to the following figures:

FIG. 1: Identification of TCR sequences expressed by antigen-specific T cells and antigens recognized by M.tb lysate-responsive T cells in controllers and progressors: Plots depicting longitudinal study time points (dots) at which PBMC samples were analyzed for each individual Controller (blue) or Progressor (red, synchronized to TB diagnosis) in the Adolescent Cohort Study (ACS) (a) or the GC6-74 cohort (b). Each horizontal line or symbol represents an individual.

FIG. 2: Identification of TCR sequences expressed by antigen-specific T cells and antigens recognized by M.tb lysate-responsive T cells in controllers and progressors: Experimental workflow and analysis approach used to identify mycobacteria reactive CDR3αβ sequences and determine their frequencies in blood. First, single cell TCR-seq (scTCR-seq) was performed on sorted mycobacteria-reactive T cells expressing the activation markers CD69 and CD154 or CD137 after in vitro M.tb lysate stimulation. GLIPH2 analysis clustered TCR sequences expressed by mycobacteria-reactive T cells into TCR similarity groups. In parallel, bulk TCR-seq was performed on PBMC (unstimulated) to profile the repertoire and determine the frequencies of CDR3β sequences in each sample. The total frequencies of CDR3β sequences within a GLIPH2 TCR similarity group was determined for each controller and progressor sample using the bulk TCR-seq data. For controllers and progressors with samples collected at multiple study timepoints, the total frequencies of CDR3β sequences within a TCR similarity group was determined for each timepoint. The total frequencies of CDR3β sequences within a TCR similarity group were compared in controllers and progressors. To identify antigens recognized by these antigen-specific T cells, transduced NFAT reporter stable J76-NFATRE-luc T-cell line cells expressing representative TCRαβ chains from TCR similarity groups found to be differentially abundant in controllers and progressors were co-incubated with artificial antigen presenting cells (aAPC) to screen for antigens representing the M.tb proteome.

FIG. 3: Analysis workflow used to measure the frequencies of GLIPH2 TCR similarity groupings from mycobacteria-reactive (M.tb) or CMV, EBV or Influenza-A (Infl.A)-specific CDR3b sequences in controllers and progressors. CDR3b sequences from mycobacteria-reactive T cells were compiled from TCR sequences expressed by sorted mycobacteria-reactive cells from controllers, progressors and healthy M.tb infected adolescents. CMV, EBV, and Infl.A-specific CDR3b sequences were obtained from VDJdb. GLIPH2 analysis was performed, and the resulting GLIPH2 similarity groups were filtered initially using the criteria listed under Filter 1. TCR similarity groups with significant HLA allele associations in the progressor/controller cohort were selected (Filter 2). Finally, similarity groups that were differentially abundant in controllers and progressors bearing the associated HLA allele were identified (Filter 3).

FIG. 4: Frequencies of CMV, EBV, Influenza A or M.tb-specific TCR specificity group: HLA combinations that associated with clinical outcome (either significantly more abundant in controllers or progressors), expressed as a percentage of all TCR specificity group: HLA combinations for that pathogen (i.e., before Filter 3 was applied in FIG. 3).

FIG. 5: Relative frequency plot of the numbers of TCR specificity group: HLA combinations found to be significantly different between the two groups. Permutation analyses were performed with 200 iterations using randomized disease outcome labels. The vertical line represents the actual number of M.tb-specific TCR specificity group: HLA combinations found to be significantly different between controllers and progressors with correct disease outcome labels.

FIG. 6: mRNA construct design: the linkers in position 1, positions 1 and 2, and/or positions 1, 2 and 3 may be selected from a nucleic acid having the nucleotide sequence of any one of SEQ ID NOs: 9-19 and the antigen sequences are selected from the codon optimised sequences encoding PE13, CFP-10, WbbL1, and PPE18 proteins in any order within the polyprotein-expressing sequence.

FIG. 7: Exemplary constructs for obtaining the mRNA vaccines of the invention: A) Construct including a codon optimised nucleotide sequence encoding each of the four antigens, PE13, CFP-10, WbbL1, and PPE18, separated by linker sequences and without a leader sequence. B) Construct including a codon optimised nucleotide sequence encoding each of the four antigens, PE13, CFP-10, WbbL1, and PPE18, separated by linker sequences and including a codon optimised nucleotide sequence encoding a leader sequence at the N-terminus.

FIG. 8: The nucleotide sequence of the nucleic acid construct provided in FIG. 7A (SEQ ID NO:30), including a codon optimised nucleotide sequence encoding PE13 (Rv1195) (SEQ ID NO:8), a linker of SEQ ID NO:11, a codon optimised nucleotide sequence encoding PPE18 (Rv1196) (SEQ ID NO:7), a linker of SEQ ID NO:16, a codon optimised nucleotide sequence encoding Wbbl1 (Rv3265c) (SEQ ID NO:5), a linker of SEQ ID NO: 14, and a codon optimised nucleotide sequence encoding CFP-10 (Rv3874) (SEQ ID NO: 6).

FIG. 9: The nucleotide sequence of the nucleic acid construct provided in FIG. 7B (SEQ ID NO:31), including a leader sequence of SEQ ID NO:25, followed by a codon optimised nucleotide sequence encoding PE13 (Rv1195) (SEQ ID NO:8), a linker of SEQ ID NO:11, a codon optimised nucleotide sequence encoding PPE18 (Rv1196) (SEQ ID NO: 7), a linker of SEQ ID NO:16, a codon optimised nucleotide sequence encoding Wbbl1 (Rv3265c) (SEQ ID NO:5), a linker of SEQ ID NO:14, and a codon optimised nucleotide sequence encoding CFP-10 (Rv3874) (SEQ ID NO:6).

FIG. 10: Size and integrity analysis of in vitro transcribed TB antigen-encoding mRNAs (monogenic) using capillary gel electrophoresis.

FIG. 11: In vitro potency assay in cultured cells. Immunofluorescence staining of HepG2 cells transfected with CFP-10 mRNA confirms in situ expression of transcripts and intracellular detection of the CFP-10 protein.

FIG. 12: Particle size analysis of formulated TB antigen-encoding mRNAs showing the size distribution by intensity. TB-antigen encoding mRNAs were formulated as lipid nanoparticles (LNPs) and sizing of LNPs encapsulating CFP-10 (A), WbbL1 (B), PE-13 (C) and PPE-18 (D) mRNAs was carried out.

FIG. 13: IFN-γ, TNF-α and IL-2 cytokine secretion profiles in ex vivo-stimulated spleen cells from immunised BALB/c mice. Increased concentrations of IFN-γ, TNF-α and IL-2 were observed in BALB/c mice immunized with WbbL1, PPE-18, and mix (CWPP) mRNA. Increased IL-2 was detected in mice immunized with PE-13 mRNA.

FIG. 14: IFN-γ, TNF-α and IL-2 cytokine secretion profiles from immunised C57BI/6 mice. Increased concentrations of IFN-γ, TNF and IL-2 were observed in C57BL/6 mice immunized with CFP-10, WbbL1, PE-13, PPE-18, and mix (CWPP) mRNA.

FIG. 15: IFN-γ, TNF-α and IL-2 cytokine secretion profiles from immunised C3HeB/FeJ mice. Increased concentrations of IFN-γ, TNF and IL-2 were observed in C3HeB/FeJ mice immunized with CFP-10 and mix (CWPP) mRNA.

FIG. 16: IL-17A and IL-22 cytokine secretion profiles from immunised C3HeB/FeJ mice. Increased concentrations of IL-17A and IL-22 were observed in C3HeB/FeJ mice immunized with CFP-10 and mix (CWPP) mRNA.

FIG. 17: Flow cytometry-based detection of IFN-γ positive CD4 T-cells. CD4 T-cells from immunised C57BL/6 mice were stimulated ex vivo with peptide pools covering the M. tb antigens CFP-10, WbbL1, PE-13 and PPE-18 and assessed for activation by IFN-γstaining. Increases in the percentage of IFN-γ +CD4+ T cells was observed following PE-13, PPE-18 and mix (CWPP) vaccinations.

FIG. 18: Flow cytometry-based detection of IFN-γ positive CD4 T-cells (A) and CD8 T-cells (B). CD4 and CD8 T-cells from immunised C3HeB/FeJ mice were stimulated ex vivo with peptide pools covering the M. tb antigens CFP-10, WbbL1, PE-13, PPE-18 or as a mixed antigen peptide pool (Peptide pool: CWPP) and assessed for activation by IFN-γ staining. Increases in the percentage of IFN-γ +CD4+ T cells was observed following CFP-10, PE-13 and mix (CWPP) mRNA vaccinations. IFN-γ +CD8+ T cells were detected following CFP-10 mRNA vaccinations.

FIG. 19: M. tb derived leader sequences L1 (SEQ ID NO:21), L2 (SEQ ID NO: 23), L3 (SEQ ID NO:25), and L4 (SEQ ID NO:27) were included in CFP-10 encoding mRNA vaccines. Antigen-specific increases in the percentage of IFN-γ +CD4+ T cells was observed in immunised C3HeB/FeJ mice when leader sequences 2, 3 and 4 were incorporated versus CFP-10 mRNA vaccines with no leader (NL) and L1-CFP-10.

FIG. 20: Figure depicting exemplary constructs used for obtaining the polyprotein mRNA vaccines of the invention.

FIG. 21: Electropherograms and capillary gel images depicting the size and integrity of two polygenic mRNA transcripts TB-M3 and TB-M4.

FIG. 22: Capillary gel images depicting the size and integrity of TB-M3 and TB-M4 polygenic mRNA transcripts with and without leader sequences (L1, L2, L3, L4, L5) and CFP-10 monogenic mRNA transcripts with and without leader sequences (L1, L2, L3, L4).

FIG. 23: TB-M7 polyprotein mRNA vaccination. Secreted cytokine profiles in ex vivo-stimulated spleen cells from mice injected intramuscularly with saline or immunized with 10 μg of LNP-formulated control mRNA encoding a bioluminescence reporter gene, or 10 μg of LNP-formulated polygenic TB-M7 mRNA. Exemplary cytokine profiles for IFN-γ and IL-2 are shown for BALB/c, C57BL6 and C3HeB/FeJ mice. Unstimulated spleen cells from TB-M7 mRNA vaccinated mice are included as a control. These results confirm individual antigen-specific T cell activation of all four TB antigens included in the polyprotein. A) Stimulation with CFP-10 peptide pools show increased concentrations of IFN-γ and IL-2 in C3HeB/FeJ mice. B) Stimulation with Wbbl1 peptide pools show increased concentrations of IFN-γ and IL-2 in BALB/c, C57BL6 and C3HeB/FeJ mice. C) Stimulation with PE-13 peptide pools show increased concentrations of IFN-γ and IL-2 in C57BL6 and C3HeB/FeJ mice. D) Stimulation with PPE-18 peptide pools show increased concentrations of IFN-γ and IL-2 in BALB/c, C57BL6 and C3HeB/FeJ mice.

FIG. 24: TB-M7 polyprotein mRNA vaccination. Exemplary secreted cytokine profiles in ex vivo-stimulated spleen cells from BALB/c, C57BL6 and C3HeB/FeJ mice stimulated with mixed antigen peptide pool (Peptide pool: CWPP). IFN-γ, IL-2, TNF-α and IL-17A cytokine concentrations are shown. BALB/c, C57BL6 and C3HeB/FeJ mice show increased IFN-γ and IL-2 following TB-M7 mRNA vaccination. Additionally, BALB/c mice show increased TNF-α and IL-17A, and C57BL6 mice show increased TNF-α.

FIG. 25: Secreted IL-6 concentrations in BALB/c, C57BL6 and C3HeB/FeJ mice vaccinated with TB-M7 polyprotein mRNA. Spleen cells were stimulated with CFP-10 (A), PE-13 (B) and PPE-18 (C) peptide pools.

FIG. 26: Secreted IL-22 concentrations in BALB/c, C57BL6 and C3HeB/FeJ mice vaccinated with TB-M7 polyprotein mRNA. Spleen cells were stimulated with WBBL1 (A), PE-13 (B) and PPE-18 (C) peptide pools.

FIG. 27: Flow cytometry-based detection of IFN-γ positive CD3 (A) and CD4 (B) T-cells in BALB/c mice vaccinated with TB-M7 polyprotein mRNA. The percentage of IFN-γ +CD3+ T cells increased following cell stimulation with WbbL1, PPE-18 and CWPP peptide pools. PPE-18 specific IFN-γ +CD4+ T cells are shown.

FIG. 28: Flow cytometry-based detection of IFN-γ positive CD4 T-cells in C57BL/6 mice vaccinated with TB-M7 polyprotein mRNA. The percentage of IFN-γ +CD4+ T cells increased following cell stimulation with WbbL1, PE-13, PPE-18 and CWPP peptide pools in C57BL6 mice.

FIG. 29: Flow cytometry-based detection of IFN-γ positive CD4 T-cells in BALB/c, C57BL/6 and C3HeB/FeJ mice vaccinated with TB-M7 polyprotein mRNA. Ex-vivo stimulation of spleen cells from vaccinated mice with a TB antigen (TB10.4) not included in the polyprotein. Flow cytometry-based detection of IFN-γ positive CD4 T-cells shows no activation of IFN-γ, confirming the results depict antigen-specific T-cell stimulation.

FIG. 30: TB-M3 and TB-M4 polyprotein mRNA vaccination. Secreted cytokine profiles in ex vivo-stimulated spleen cells from mice injected intramuscularly with 10 μg of LNP-formulated control mRNA encoding a bioluminescence reporter gene (control), or 10 μg of LNP-formulated polygenic TB-M3 or TB-M4 mRNA. Exemplary cytokine profiles for IFN-γ and IL-2 are shown for BALB/c, C57BL6 and C3HeB/FeJ mice. These results confirm individual antigen-specific T cell activation of all four TB antigens included in the polyprotein. A) Stimulation with CFP-10 peptide pools show increased concentrations of IFN-γ and IL-2 in C3HeB/FeJ mice. B) Stimulation with Wbbl1 peptide pools show increased concentrations of IFN-γ in and IL-2 in BALB/c, C57BL6 and C3HeB/FeJ mice.C) Stimulation with PE-13 peptide pools show increased concentrations of IFN-γ and IL-2 in C57BL6 and C3HeB/FeJ mice. D) Stimulation with PPE-18 peptide pools show increased concentrations of IFN-γ in BALB/c an C57BL6 mice and IL-2 in BALB/c, C57BL6, and C3HeB/FeJ mice.

### SEQUENCE LISTING

The nucleic acid and amino acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:
SEQ ID NO:1—amino acid sequence of Wbbl1 (Rv3265c)
SEQ ID NO:2—amino acid sequence of CFP-10 (Rv3874)
SEQ ID NO:3—amino acid sequence of PPE18 (Rv1196)
SEQ ID NO:4—amino acid sequence of PE13 (Rv1195)
SEQ ID NO:5—codon optimised nucleotide sequence of Wbbl1 (Rv3265c)
SEQ ID NO:6—codon optimised nucleotide sequence of CFP-10 (Rv3874)
SEQ ID NO:7—codon optimised nucleotide sequence of PPE18 (Rv1196)
SEQ ID NO:8—codon optimised nucleotide sequence of PE13 (Rv1195)
SEQ ID NO:9—nucleotide sequence of linker 1
SEQ ID NO: 10—nucleotide sequence of linker 2
SEQ ID NO:11—nucleotide sequence of linker 3
SEQ ID NO:12—nucleotide sequence of linker 4
SEQ ID NO:13—nucleotide sequence of linker 5
SEQ ID NO:14—nucleotide sequence of linker 6
SEQ ID NO: 15—nucleotide sequence of linker 7
SEQ ID NO: 16—nucleotide sequence of linker 8
SEQ ID NO:17—nucleotide sequence of linker 9
SEQ ID NO:18—nucleotide sequence of linker 10
SEQ ID NO:19—nucleotide sequence of linker 11
SEQ ID NO:20—amino acid sequence of M.tb Rv2878c derived leader
SEQ ID NO:21—codon optimised nucleotide sequence of M.tb Rv2878c derived leader
SEQ ID NO:22—amino acid sequence of M.tb MT18B_2507 derived leader
SEQ ID NO:23—codon optimised nucleotide sequence of M.tb MT18B_2507 derived leader
SEQ ID NO:24—amino acid sequence of M.tb Rv3803c derived leader
SEQ ID NO:25—codon optimised nucleotide sequence of M.tb Rv3803c derived leader
SEQ ID NO:26—amino acid sequence of M.tb Rv1860 derived leader
SEQ ID NO:27—codon optimised nucleotide sequence of M.tb Rv1860 derived leader
SEQ ID NO: 28—amino acid sequence of Human interleukin 2 derived leader
SEQ ID NO:29—codon optimised nucleotide sequence of Human interleukin 2 derived leader
SEQ ID NO:30—nucleotide sequence of exemplary construct without leader
SEQ ID NO:31—nucleotide sequence of exemplary construct with leader
SEQ ID NO:32—stop codon nucleotide sequence
SEQ ID NO:33—amino acid sequence of TB-M3 polyprotein
SEQ ID NO:34—amino acid sequence of TB-M4 polyprotein
SEQ ID NO:35—amino acid sequence of TB-M7 polyprotein
SEQ ID NO:36—amino acid sequence of TB-M8 polyprotein
SEQ ID NO:37—codon optimised nucleotide sequence of TB-M3 polygenic construct
SEQ ID NO:38—codon optimised nucleotide sequence of TB-M4 polygenic construct
SEQ ID NO:39—codon optimised nucleotide sequence of TB-M7 polygenic construct
SEQ ID NO:40—codon optimised nucleotide sequence of TB-M8 polygenic construct

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., in some embodiments “comprising” is to be understood as having the meaning of “consisting of”.

The inventors of the present invention have developed an mRNA vaccine construct that contains a unique combination of M.tb proteins, which were found to be associated with controlled M.tb infection in humans. The invention is a messenger RNA (mRNA)-based vaccine that induces an immune response to tuberculosis (TB). The vaccine is a mRNA expression cassette, encoding a mono/polyprotein of the M.tb proteins, Rv3265c (WbbL1), Rv1195 (PE13), Rv1196 (PPE18), and Rv3874 (CFP-10) and generates an adaptive immune response specific to these proteins. To the inventors' knowledge, the selection of antigens for all other vaccine candidates for TB has not been based on knowledge of controlled M.tb infection in humans, because such knowledge has been lacking until now. The present mRNA vaccine construct comprises modified bacterial RNA sequences encoding the amino acid sequences of WbbL1, PE13, PPE18, and CFP-10, which were codon optimised for mammalian expression and immune recognition. The nanoparticle formulation of the vaccine construct further uses a novel renewable lipid formulation to incorporate the RNA sequences.

Specifically, the selection of the proteins included in the vaccine construct was informed by results which identified T cell responses and their protein targets that were associated with controlled M.tb infection in humans. The inventors of the present invention used single cell and bulk TCR sequencing and the GLIPH2 algorithm to analyze millions of M.tb-specific sequences in two longitudinal cohorts, comprising 184 individuals with M.tb infection who either progressed to tuberculosis (TB) disease (progressors) or controlled infection (controllers). They found dozens of T cell groups with similar TCRB sequences, predicted by GLIPH2 to have common antigen specificities, which associated with control of infection, and others associated with progression to disease. Using a genome-wide M.tb antigen screen, they identified antigens and the peptides targeted by T cell similarity groups enriched either in controllers or progressors.

This allowed the inventors to rationally select four antigens for vaccine development among the ˜4000 proteins expressed by M.tb. Furthermore, the inventors have modified the RNA sequences encoding the selected WbbL1, PE13, PPE18, and CFP-10 proteins to optimise expression and uptake of the vaccine antigens in human cells.

Other researchers have identified vaccine antigens by looking at which M.tb proteins are recognised by infected people or which M.tb proteins induce protective responses in animal models of TB. To the inventors' knowledge this is the first time that samples from human controllers and progressors were used to measure T cell responses associated with controlled infection and identification of the specific proteins expressed by M.tb that these T cells target.

Traditionally, antigen discovery for vaccine development starts with the most immunogenic antigens from a given pathogen. A number of M.tb antigens used in candidate TB vaccines have been identified in this way. However, M.tb expresses roughly 4000 gene products and it remains hypothetical that the most immunogenic antigens in natural infection are the most critical immunological targets for disease control, especially as an important resistance strategy for pathogens is to avoid expulsion before transmission. By combining the power of new sequencing and TCR analysis methods with clinically relevant cohorts, the inventors employed an entirely new strategy to profile the αβ TCR response repertoire to M.tb between controllers and progressors without prescribing the antigens involved, and focussed on those TCR specificities that associated with clinical outcome.

Immunogenicity analyses of the mRNA vaccine constructs have been assessed in three different mouse strains, BALB/c, C57BL/6, and C3HeB/FeJ mice. The results confirm that mRNA vaccine delivery by intramuscular administration induces T cell responses to all four antigen components of the mRNA vaccine constructs in genetically different mouse strains. These immune responses primarily comprise CD4 T cells that express Th1 cytokines (IFNγ, TNF and IL-2) as well as some IL-17 and IL-22, although IFN-γ-expressing CD8 T cell responses are also induced. Since Th1 CD4 T cell responses are necessary for protective immunity against TB, these results suggest that these mRNA vaccine constructs are likely to protect against M. tuberculosis challenge.

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation).

An “antigen” is a compound, composition, or substance that can stimulate the production of antibodies and/or a CD4+ or CD8+ T cell response in an animal or human, including compositions that are injected or absorbed into an animal or human. An antigen reacts with the products of specific humoral or cellular immunity, including those induced by heterologous immunogens. The term “antigen” includes all related antigenic epitopes. An “epitope” refers to a site on an antigen, including chemical groups or peptide sequences on a molecule that are antigenic, i.e., that elicit a specific immune response. An antibody specifically binds a particular antigenic epitope on a polypeptide.

The terms “nucleic acid”, “nucleic acid molecule” and “polynucleotide” are used herein interchangeably and encompass both ribonucleotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase). Accordingly, a “cDNA clone” refers to a duplex DNA sequence which is complementary to an RNA molecule of interest, and which is carried in a cloning vector.

The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

It is generally understood by those of skill in the art that DNA may be transcribed to mRNA through a process of in vitro or in vivo transcription. Alternatively, RNA may be synthetically obtained by a process known in the art based on a corresponding DNA sequence. DNA sequences provided herein are interchangeable with RNA sequences, wherein the thymidine residues of the DNA sequence are replaced with uracil residues in the corresponding RNA sequence.

In some embodiments, nucleic acid constructs of the invention may include, without limitation, nucleotide sequences encoding antigenic peptides including amino acid sequences substantially identical to the amino acid sequences of the M.tb proteins Rv3265c (WbbL1), Rv1195 (PE13), Rv1196 (PPE18), and Rv3874 (CFP-10). Another embodiment of the invention includes, without limitation, nucleic acid molecules encoding the aforementioned antigenic peptides that are substantially identical to the nucleotide sequences described herein.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the antigenicity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Those skilled in the art will appreciate that polypeptides, peptides or peptide analogues can be synthesised using standard chemical techniques, for instance, by automated synthesis using solution or solid phase synthesis methodology. Automated peptide synthesisers are commercially available and use techniques known in the art. Polypeptides, peptides and peptide analogues can also be prepared from their corresponding nucleic acid molecules using recombinant DNA technology.

As used herein, the term “gene” refers to a nucleic acid that encodes a functional product, for instance an RNA, polypeptide or protein. A gene may include regulatory sequences upstream or downstream of the sequence encoding the functional product.

As used herein, the term “coding sequence” refers to a nucleic acid sequence that encodes a specific amino acid sequence. On the other hand, a “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences.

In some embodiments, the genes used in the method of the invention may be operably linked to other sequences. By “operably linked” is meant that the nucleic acid molecules encoding the recombinant polypeptides of the invention and regulatory sequences are connected in such a way as to permit expression of the proteins when the appropriate molecules are bound to the regulatory sequences. Such operably linked sequences may be contained in vectors or expression constructs which can be transformed or transfected into host cells for expression. It will be appreciated that any vector or vectors can be used for the purposes of expressing the recombinant antigenic polypeptides of the invention.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene in different cell types, or at different stages of development, or in response to different environmental or physiological conditions. A “constitutive promoter” is a promoter that direct the expression of a gene of interest in most host cell types most of the time.

The term “recombinant” means that something has been recombined. When used with reference to a nucleic acid construct the term refers to a molecule that comprises nucleic acid sequences that are joined together or produced by means of molecular biological techniques. The term “recombinant” when used in reference to a protein or a polypeptide refers to a protein or polypeptide molecule which is expressed from a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e., by human intervention. Recombinant nucleic acid constructs may be introduced into a host cell by transformation. Such recombinant nucleic acid constructs may include sequences derived from the same host cell species or from different host cell species.

The term “vector” refers to a means by which polynucleotides or gene sequences can be introduced into a cell. There are various types of vectors known in the art including plasmids, viruses, bacteriophages and cosmids. Generally, polynucleotides or gene sequences are introduced into a vector by means of a cassette. The term “cassette” refers to a gene sequence or gene sequences inserted into a vector, which in some embodiments, provides regulatory sequences for expressing the polynucleotide or gene sequences. In other embodiments, the vector provides the regulatory sequences for the expression of the polypeptides of the invention. In further embodiments, the vector provides some regulatory sequences, and the nucleotide or gene sequence provides other regulatory sequences.

The constructs of the present invention can be provided either alone or in combination with other compounds (for example, nucleic acid molecules, small molecules, peptides, or peptide analogues), in the presence of a liposome, an adjuvant, or any carrier, such as a pharmaceutically acceptable carrier and in a form suitable for administration to mammals, for example, humans, cattle, sheep, etc.

As used herein a “pharmaceutically acceptable carrier” or “excipient” includes any and all antibacterial and antifungal agents, coatings, dispersion media, solvents, isotonic and absorption delaying agents, and the like that are physiologically compatible. A “pharmaceutically acceptable carrier” may include a solid or liquid filler, diluent or encapsulating substance which may be safely used for the administration of the recombinant vaccine construct to a subject. The pharmaceutically acceptable carrier can be suitable for intramuscular, intradermal, intravenous, intraperitoneal, subcutaneous, oral or sublingual administration. Pharmaceutically acceptable carriers include sterile aqueous solutions, dispersions and sterile powders for the preparation of sterile solutions. The use of media and agents for the preparation of pharmaceutically active substances is well known in the art. Where any conventional media or agent is incompatible with the active compound, use thereof in the pharmaceutical compositions of the invention is not contemplated. Supplementary active compounds can also be incorporated into the compositions.

Suitable formulations or compositions to administer the vaccine construct of the present invention to a subject also fall within the scope of the invention. Any appropriate route of administration may be employed, such as, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, aerosol, topical, or oral administration.

Typically, an effective amount of the vaccine construct or a formulation or composition comprising the vaccine construct will be administered to a subject. As used herein the term “subject” includes all mammals, and in particular a human subject.

The invention also relates in part to a method of eliciting an immune response against M.tb or treating a M.tb infection or TB disease in a subject comprising administering to a subject in need thereof a therapeutically effective amount, an immunogenically effective amount, or a prophylactically effective amount of the constructs or compositions or formulations thereof of the present invention, in order to prevent or treat TB in the subject.

Vaccine formulations and compositions that are useful in the present invention include the polygenic or monogenic nucleic acid constructs described herein, preferably mRNA constructs, and/or lipid nanoparticles include the nucleic acid constructs that prime and/or boost an immune response to M.tb.

In one embodiment, the vaccine formulations are capable of “priming” an immune response to M.tb. In a further embodiment, the vaccine formulations are capable of “boosting” an immune response to M.tb, for example where a subject has already received a priming vaccine. It will further be appreciated that a “boost” composition may include nucleic acid constructs or vaccine formulations which are administered to the subject in two or more doses after an initial priming inoculation.

An “effective amount” of the vaccine construct and/or composition of the present invention includes a therapeutically effective amount, immunologically effective amount, or a prophylactically effective amount. A “therapeutically effective amount” refers to an amount effective, at dosages and for periods of time necessary, to achieve the desired therapeutic result, such as treatment of a M.tb infection or a condition associated with such infection. A therapeutically effective amount of a compound may vary according to factors such as the disease state, age, sex, and weight of the individual, and the ability of the composition to elicit a desired response in the individual. Dosage regimens may be adjusted to provide the optimum therapeutic response. A therapeutically effective amount is also one in which any toxic or detrimental effects of the composition are outweighed by the therapeutically beneficial effects.

The dosage of the vaccine construct or pharmaceutical compositions of the present invention will vary depending on the symptoms, age and body weight of the subject, the nature and severity of the disorder to be treated or prevented, the route of administration, and the form of the composition. Any of the compositions of the invention may be administered in a single dose or in multiple doses. The dosages of the compositions of the invention may be readily determined by techniques known to those of skill in the art or as taught herein.

By “immunogenically effective amount” is meant an amount effective, at dosages and for periods of time necessary, to achieve a desired immune response. The desired immune response may include stimulation or elicitation of a protective immune response, for instance a T-cell response.

A “prophylactically effective amount” refers to an amount effective, at dosages and for periods of time necessary, to achieve a desired prophylactic result, such as inducing protective immunity against a M.tb infection. Typically, a prophylactic dose is used in a subject prior to or at an earlier stage of infection, so that a prophylactically effective amount may be less than a therapeutically effective amount.

The amount of mRNA construct in the composition may vary according to factors such as the infection state, age, sex, and weight of the individual. Dosage regimens may be adjusted to provide the optimum therapeutic response. For example, a single dose may be administered, or multiple doses may be administered over time. It may be advantageous to formulate the compositions in dosage unit forms for ease of administration and uniformity of dosage.

The term “preventing”, when used in relation to an infectious disease, such as TB, is well understood in the art, and includes administration of a composition which either reduces the frequency of or delays infection with the organism (prevention of infection vaccine), or in those already infected with the organism, reduces the frequency of or delays progression to disease, onset of pathology or symptoms of a condition (prevention of disease) in a subject relative to a subject who does not receive the composition. Prevention of a disease includes, for example, reducing the severity of pathology, or the number of diagnoses of the disease, in a treated population versus an untreated control population, and/or delaying the onset of progression to disease, symptoms of the disease in a treated population versus an untreated control population.

The term “treating”, or “treatment” includes administering to a subject in need thereof a therapeutically effective amount of a construct or composition of the invention and includes both prophylactic and/or therapeutic treatment with the constructs or compositions. The treatment may be in combination with another agent, including one or more antibiotics, to diminish, ameliorate, or accelerate recovery from the TB disease or to prevent recurrent TB disease.

The term “prophylactic and/or therapeutic” treatment is well known to those of skill in the art and includes administration to a subject of one or more of the compositions of the invention. If the composition is administered prior to infection with the organism, or prior to clinical manifestation of the unwanted condition (e.g., disease or other unwanted state of the subject) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The vaccination protocol for eliciting an immune response against Mycobacterium tuberculosis in a subject as defined herein typically comprises a series of single doses of the nucleic acid constructs or compositions described herein. A single dose or dosage, as used herein, refers to the priming dose (i.e., initial first or second dose with the same antigens), and any subsequent dose, respectively, which are preferably administered in order to “boost” the immune reaction. In this context, each single dosage comprises the administration of one of the antigens or compositions according to the invention, wherein the interval between the administration of two single dosages can vary from at least one week, preferably 2, 3, 4, 5, 6, 7, 8, 9, 10, 11 or 12 weeks apart. Most preferably, the antigens or compositions of the invention are administered at intervals of either 4 or 8 weeks apart. It will be appreciated that the intervals between single dosages may be constant or vary over the course of the immunization protocol, e.g., the intervals may be shorter in the beginning (such as 4 weeks apart) and longer towards the end of the protocol (such as 8 weeks apart). Additionally, depending on the total number of single dosages and the interval between single dosages, the immunization protocol may extend over a period of time, which preferably lasts at least one week, more preferably several weeks, even more preferably several months (e.g., 3, 4, 5, 6, 7, 8, 9, 10, 11, 12, 18 or 24 months). Each single dosage encompasses the administration of one of the antigens or mRNA vaccine constructs described herein.

Toxicity and therapeutic efficacy of compositions of the invention may be determined by standard pharmaceutical procedures in cell culture or using experimental animals, such as by determining the LD₅₀ and the ED₅₀. Data obtained from the cell cultures and/or animal studies may be used to formulate a dosage range for use in a subject. The dosage of any composition of the invention lies preferably within a range of concentrations that include the ED₅₀ but which has little or no toxicity. The dosage may vary within this range depending upon the dosage form employed and the route of administration utilised. For compositions of the present invention, the therapeutically effective dose may be estimated initially from cell culture assays.

In some embodiments, the vaccine constructs or compositions according to the invention may be provided in a kit, optionally with a carrier and/or an adjuvant, together with instructions for use.

The following examples are offered by way of illustration and not by way of limitation.

### Example 1

### Identification of Antigens Involved in the αβ T Cell Response to Mycobacterium Tuberculosis Infection

Defining M.tb-Specific T Cells and their Repertoires

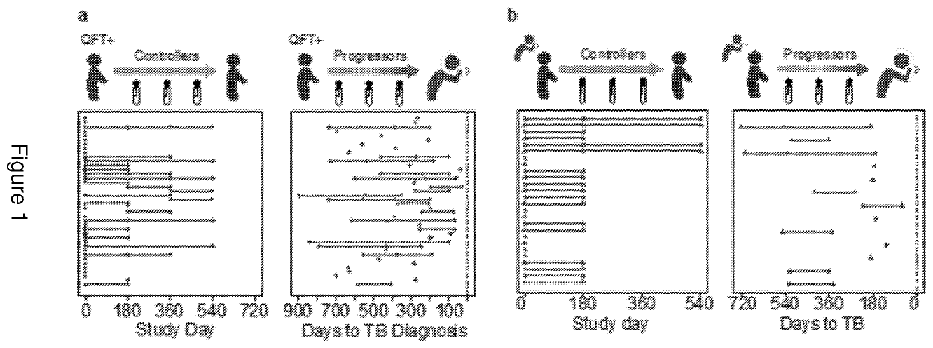

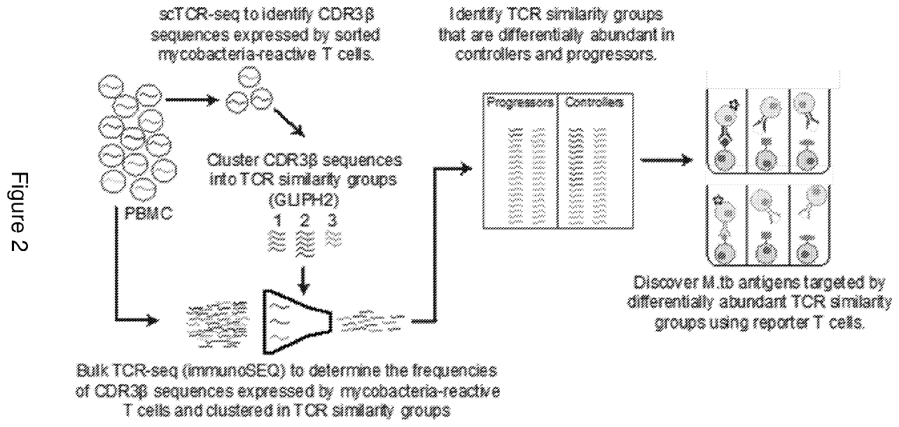

The inventors first determined TCRαβ sequences expressed by mycobacteria-reactive T cells in in adolescents who controlled M.tb infection (“controllers”) and those who progressed to TB disease (“progressors”). The adolescents were selected from those with evidence of M.tb infection who participated in the Adolescent Cohort Study (ACS), a large epidemiological study of TB. Progressors (n=44) developed microbiologically-confirmed, intrathoracic TB during 2 years of follow-up. Controllers (n=44) also had evidence of M.tb infection but did not develop TB during follow-up. Mycobacteria-reactive T cells were identified by stimulating thawed PBMC from progressors and controllers with M.tb-lysate, comprising both protein and non-protein antigens, and sorting activated CD4 or CD8 T cells (FIG. 1 and FIG. 2). Activated T cells were identified by their elevated expression levels of CD69 together with CD154 or CD137 for single cell TCR sequencing as described in Huang et al (2020). The TCRαβ repertoire of M.tb-lysate responsive T cells was successfully captured from PBMC samples collected from 35 controllers and 35 progressors using this single cell TCR sequencing approach. Among 37,674 sorted T cells from progressors and controllers, 22,276 (59.1%) CDR3× and 21,404 (56.8%) CDR3B sequences were detected, of which 15,272 and 16,517 were unique, respectively. Higher frequencies of activated T cells were observed following stimulation with M.tb lysate compared to PBS, but frequencies of activated T cells between controllers and progressors were not different, nor were numbers of CDR3β sequencies detected. Additionally, frequencies of activated T cells were constant over the two-year follow up period. Clonal expansions (>2 clones) were observed in single cell TCR data from all but four samples. More than 90% of sorted M.tb lysate-reactive T cells were CD4 T cells, 2.2% were CD8 T cells and 6.5% expressed canonical mucosal-associated invariant T (MAIT) cell CDR3a sequences irrespective of CD4 and CD8 expression. Cells expressing known canonical MAIT CDR3α sequences expressed markedly higher levels of CD26, a marker associated with MAIT cells, compared with CD4 and CD8 T cells, demonstrating that the phenotype of single-cell sorted cells faithfully aligns with the TCR identity. Expected levels of mRNA expression of known functional markers by sorted CD4, CD8 and MAIT cells further validated the experimental TCR sequencing pipeline used. For example, a higher proportion of M.tb-lysate responsive MAIT cells expressed IFNG mRNA compared to CD4 and CD8 T cells, while a higher proportion of CD4 T cells expressed TNF, IL2, IL17A and IL13 than CD8 and MAIT cells, and higher proportions of CD8 T cells and MAIT cells expressed EOMES and PERF, than CD4 T cells. However, the proportions of T cells expressing any of 20 functional transcripts previously measured to assess T cell responses to M.tb lysate (Han et al, 2014) were not significantly different between controllers and progressors. Since T cells expressing a Th1/Th17 phenotype have been implicated in control of M.tb, the inventors compared proportions of Th1/Th17 T cells, defined as those expressing at least two Th1 genes (i.e., IFNG, TNF, IL2 or TBX21) plus IL17A or RORC, in controllers and progressors; proportions of these cells in controllers were also not different.

### Comparison of M.tb TCR Similarity Groups in Single-Cell Repertoires

The inventors then combined the CDR3β sequences obtained from mycobacteria-reactive CD4 T cells from controllers, progressors, and previously published TCR datasets (Huang et al, 2020 and Glanville et al, 2017), amounting to 25,256 CDR3β sequences. To determine if M.tb-specific T cells are preferentially enriched at the site of recent or ongoing TB disease, bulk TCR data generated from blood and resected lung tissue samples, collected from an independent cohort of TB patients was compared. M.tb-lysate reactive CD4 TCR sequences were significantly enriched in lung tissue compared with corresponding peripheral blood samples. By contrast, frequencies of CMV, EBV, and Influenza A-specific CDR3β sequences were not different between blood and lung resection samples, consistent with an expansion of M.tb-specific TCRs at the site of recent or on-going disease.

The incredible diversity and private nature of CDR3β sequences have necessitated the development of clustering methods that group CDR3β sequences which likely share epitope specificities. Such clustering methods allow inter-individual comparisons of CDR3β sequences which likely share antigen specificity. The inventors sought to determine if such clusters of TCRs were differentially associated with either controllers or progressors. Using GLIPH2 (Huang et al, 2020) to cluster TCRb sequences expressed by mycobacteria-reactive T cells, they identified 3,417 M.tb TCR similarity groups. 54% of TCR similarity groups contained CDR3b sequences observed in sorting experiments performed in at least two independent studies (Huang et al, 2020 and Glanville et al, 2017). This observation strongly implies that most of the TCR similarity groups contained TCRs that target antigens in M.tb lysate.

In Huang et al (2020) it was reported that applying filters to the GLIPH2 output parameters narrowed down the number of TCR similarity groups and enriches for groups more likely to have been clustered correctly. The inventors thus selected TCR similarity groups shared by >3 participants, consistent of >3 unique CDR3b sequences, with enriched common V-genes (vb_score<0.05), with a limited CDR3 length distribution (length_score<0.05) and with statistically significant motifs from a reference set of CDR3b sequences (Fisher_score<0.05). This filtering resulted in 290 TCR similarity groups. They then investigated whether any of the selected TCR similarity groups were significantly enriched in sorted M.tb lysate-reactive CD4 T cells from controllers or progressors. The majority of TCR similarity groups were shared between controllers and progressors, suggesting a high degree of overlap in T cell specificities between the groups. However, some TCR similarity groups appeared to be enriched in one group. The degree of TCR sequence diversity may be associated with control of M.tb, or, alternatively, may be associated with progression. The large size of the M.tb TCR sequence dataset enabled assessment of TCR similarity group diversity within individuals.

### Controller and Progressor-Associated M.tb TCR Similarity Groups in Bulk TCR Repertoires

Single cell TCR sequencing of M.tb-specific cells was necessary for identifying TCR similarity groups likely to target M.tb antigens and to identify TCRα and TCRβ pairs that allow establishment of peptide-MHC specificity. However, single cell TCR sequencing does not allow accurate quantitation of clonotypes within the overall TCR repertoire in peripheral blood. To estimate relative frequencies of individual TCR sequences expressed by mycobacteria-reactive T cells, bulk TCRB repertoire profiling was performed in unstimulated PBMC samples from a subset of ACS study participants (n=30) who had remaining PBMC samples following single cell sorting, and, in a second longitudinal cohort of adult progressors and controllers enrolled into the Grand Challenges 6-74 (GC6-74). The GC6-74 cohort comprised South African household contacts of TB patients who either developed microbiologically confirmed pulmonary TB (progressors, n=12) or remained healthy (controllers, n=25) (FIG. 1). From the combined ACS and GC6-74 bulk TCR sequencing data only CDR3b sequences associated with mycobacteria-reactive T cells (i.e. CDR3b expressed in sorted mycobacteria-reactive T cells) were selected.

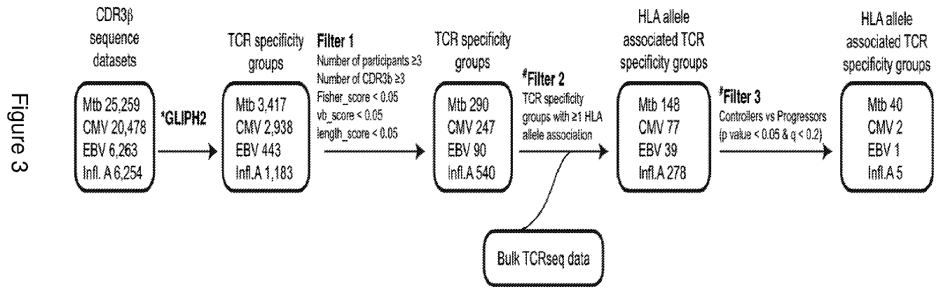

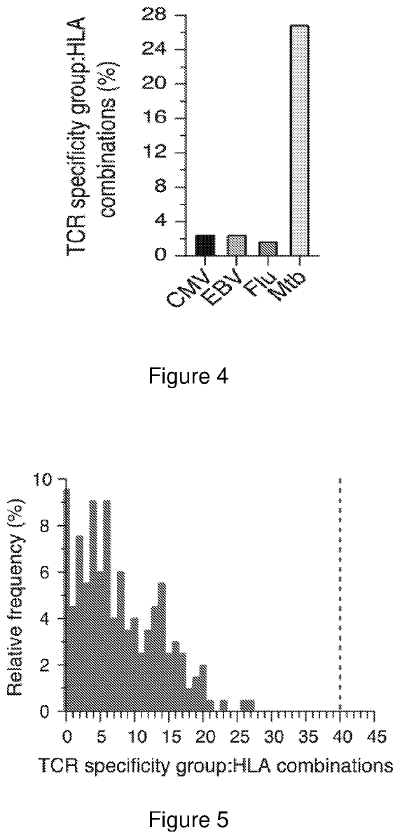

From 290 mycobacteria-reactive TCR similarity groups initially filtered on GLIPH2 output parameters, further TCR similarity groups were selected that had a significant HLA association using a Fisher's exact test p-value threshold of <0.05 (HLA alleles defined by two-digit typing). Among 148 TCR similarity groups: HLA allele combinations that met this critetion, frequencies of TCRs belonging to each similarity group in unstimulated PBMC samples from controllers and progressors bearing the associated HLA allele were compared. A total of 40 TCR similarity group: HLA allele combinations, comprising 25 GLIPH2 TCR similarity groups, were differentially abundant in controllers and progressors at a p-value threshold below 0.05, after controlling the false discovery rate using the Benjamini Hochberg method (q<0.2). Thirty TCR similarity group: HLA allele combinations had higher frequencies in controllers than progressors, while 10 TCR similarity group: HLA allele combinations were more abundant in progressors. Among these, the DRB1*15-associated TCR similarity group “SVAL” was highly enriched in controllers, consistent with the finding of higher diversity of TCRs among DRB1*15-expressing controllers. To investigate the specificity of the disease-outcome associated TCR similarity groups, the inventors compared frequencies of CMV, EBV, and Influenza A TCR similarity groups, identified using the GLIPH2-based pipeline (FIG. 3) in controllers and progressors. Two CMV, 1 EBV, and 5 influenza A-specific TCR similarity groups were differentially abundant between controllers and progressors (FIG. 3 and FIG. 4). To test if outcome-associated M.tb-reactive TCR similarity group: HLA allele combinations were non-random, permutation analyses were performed using randomized disease outcome labels and the number of significantly associated clusters from 200 iterations was determined. The 40 M.tb-specific GLIPH2 specificity groups associated with clinical outcome significantly exceeded the numbers obtained from the 200 iterations with randomized disease outcome (FIG. 5). Further, the number of identified CMV, EBV, or Flu-specific TCR groups fell well within the distribution obtained from the analysis with randomised outcome label. These results validate the specificity of the outcome-associated M.tb-reactive TCR group discovery approach and suggest the TCR groups identified were non-random.

The inventors also sought to investigate the longitudinal kinetics of differentially abundant TCR similarity groups in samples collected at various time points before TB diagnosis in progressors or throughout study follow up in controllers, modeled by fitting non-linear splines. Overall, these analyses yielded large 95% confidence intervals, highlighting the high degree of inter-sample and inter-individual heterogeneity of M.tb-specific TCR data. However, the results suggest that for many of the clusters identified to be more frequent in controllers, the TCRs were elevated in controllers throughout the study period. Similarly, TCR clusters identified to be more frequent in progressors were also generally elevated in progressors throughout the study period.

To determine if these results were robust to the TCR clustering algorithm, the outcome-associated TCR similarlity group discovery analysis was repeated using TCRdist3, another clustering algorithm. The TCRdist3 pipeline identified 246 unique mycobacteria-reactive metaclone clusters with significant HLA allele associations. Of these, 46 metaclone cluster: HLA allele combinations consisting of 33 unique metaclone clusters were differentially abundant in controllers and progressors. Overall, 64% of GLIPH2-identified clusters associated with clinical outcome were also identified by TCRdist3, while 44.4% of all clinical outcome-associated clusters identified by either GLIPH2 or TCRdist3 were identified by both. This substantial overlap between differentially abundant clusters identified using GLIPH2 or TCRdist3 analysis pipelines suggested that these results are largely independent of TCR clustering algorithm.

### Example 2

### Identifying Epitopes Targeted by Controller and Progressor Associated TCR Similarity Groups

Next, the inventors sought to identify antigens and epitopes targeted by TCRs that belong to differentially abundant GLIPH2 TCR similarity groups (i.e., similarity groups associated with controllers or similarity groups associated with progressors).

Selection of the 4 M.tb proteins that comprise the mRNA vaccine construct was guided by data that was generated in Example 1 where the inventors measured T cell responses and their antigenic targets in controllers and progressors. It is important to note that peripheral blood mononuclear cells (PBMCs) were collected from progressors before TB disease diagnosis. Therefore, the inventors were able to compare frequencies of antigen-specific T cell responses in controllers and progressors up to 24 months before the onset of clinical disease. FIG. 1 and FIG. 2 depict the study design and summary of the experimental approach used to identify the M.tb proteins preferentially recognised by controllers. The mRNA vaccine construct of the present invention comprises modified bacterial RNA sequences encoding the amino acid sequences of WbbL1, PE13, PPE18, and CFP-10, which were codon optimised for mammalian expression and immune recognition.

As detailed in Example 1, T cell clones that responded to M.tb in controllers and progressors were first identified. This was done by stimulating PBMCs with M.tb lysate, an antigen preparation containing M.tb proteins, lipids and carbohydrates, for 12 hours. Using flow cytometry, the inventors identified and sorted single TCRαβ+ T cells that co-expressed the activation markers CD69+CD137+ and/or CD69+CD154+ into 96 well plates. The plates containing One-Step RT-PCR buffer (Qiagen) and a panel of primers were then subjected to TCRαβ sequence-specific amplification in a nested PCR before sequencing the TCR on a MiSeq (Illumina) instrument as described in Han et al (2014), Glanville et al (2017) and Huang et al (2020). A clustering algorithm, known as GLIPH2 (Huang et al, 2020), was used to group T cell clones that were predicted based on their TCR CDR3β amino acid motifs to recognise the same M.tb proteins. These TCR groups are known as GLIPH2-specificity clusters. Next, a complementary sequencing approach, known as bulk TCR sequencing (Adaptive Biotechnologies immmunoSEQ), was used to measure frequencies of these individual TCRs in PBMC samples collected from controllers and progressors, providing an estimate of the abundance of antigen-specific T cells in peripheral blood. The inventors then assessed if certain GLIPH2-specificity clusters were more abundant in controllers compared to progressors and identified several clusters, or T cell responses, that were significantly more abundant in controllers (protective T cells) as well as some clusters that were significantly more abundant in progressors (pathogenic T cells).

For a subset of these GLIPH2-specificity clusters, the inventors identified the M.tb proteins that are recognised by T cells that belong to these GLIPH2-specificity clusters. This was done by cloning representative TCRα and β chains from a specific GLIPH2-specificity cluster into a NFAT reporter stable J76-NFATRE-luc T-cell line, which is deficient for both TCRα and TCRβ chains as described in Huang et al (2020), to allow expression of the TCR, thus conferring antigen specificity to the recombinant T cell line. Artificial antigen presenting cells (aAPC) were then constructed using lentiviral transduction of CD80 and HLA-DM molecules into K562 cells, as well as candidate HLA alleles that correspond to the HLA alleles from donors who possessed the GLIPH2-specificity clusters. This allowed for the identification that GLIPH2-specificity clusters, or protective T cells, which recognised PE13, CFP-10, WbbL1, and PPE18 were more abundant in controllers compared to progressors. These proteins were then incorporated into the mRNA vaccine construct.

### Example 3

Designing the mRNA Vaccine Constructs
mRNA Sequence Modifications to Codon Optimise Antigen Expression in Mammalian Cells, Humoral and T-Cell Immune Responses and Subvert Induction of Strong Type I IFN Responses

Codon optimised and uridine-depleted sequences encoding the bacterial PE13, CFP-10, WbbL1, and PPE18 proteins have been designed, synthesized and cloned into plasmid templates to be used for in vitro transcription (IVT). Codon optimisation and uridine depletion are useful to enhance protein expression and attenuate innate immunostimulatory effects (type I IFN responses) respectively.

The amino acid sequences of the PE13, CFP-10, WbbL1, and PPE18 proteins are provided in Table 1. The codon optimised and uridine depleted nucleic acid sequences encoding the PE13, CFP-10, WbbL1, and PPE18 proteins designed for use in the mRNA vaccine are provided in Table 2.

TABLE 1
Amino acid sequences of the PE13, CFP-10, WbbL1, and PPE18
proteins.

| M.tb | | |
|---|---|---|
| antigenic | | SEQ ID |
| protein | Amino acid sequence* | NO |
| Wbbl1 | WVAVTYSPGPHLERFLASLSLATERPVSVLLADNGSTDGTPQAAVQRY | SEQ ID |
| (Rv3265c) | PNVRLLPTGANLGYGTAVNRTIAQLGEMAGDAGEPWVDDWVIVANPD | NO: 1 |
| 33.27 kDa | VQWGPGSIDALLDAASRWPRAGALGPLIRDPDGSVYPSARQMPSLIR | |
| | GGMHAVLGPFWPRNPWTTAYRQERLEPSERPVGWLSGSCLLVRRSA | |
| | FGQVGGFDERYFMYMEDVDLGDRLGKAGWLSVYVPSAEVLHHKAHS | |
| | TGRDPASHLAAHHKSTYIFLADRHSGWWRAPLRWTLRGSLALRSHLM | |
| | VRSSLRRSRRRKLKLVEGRH | |
| CFP-10 | AEMKTDAATLAQEAGNFERISGDLKTQIDQVESTAGSLQGQWRGAAG | SEQ ID |
| (Rv3874) | TAAQAAVVRFQEAANKQKQELDEISTNIRQAGVQYSRADEEQQQALS | NO: 2 |
| 10.8 kDa | SQMGF | |
| PPE18 | VDFGALPPEINSARMYAGPGSASLVAAAQMWDSVASDLFSAASAFQS | SEQ ID |
| (Rv1196) | WWWGLTVGSWIGSSAGLMVAAASPYVAWMSVTAGQAELTAAQVRVA | NO: 3 |
| 39.17 kDa | AAAYETAYGLTVPPPVIAENRAELMILIATNLLGQNTPAIAVNEAEYGEM | |
| | WAQDAAAMFGYAAATATATATLLPFEEAPEMTSAGGLLEQAAAVEEA | |
| | SDTAAANQLMNNVPQALQQLAQPTQGTTPSSKLGGLWKTVSPHRSPI | |
| | SNMVSMANNHMSMTNSGVSMTNTLSSMLKGFAPAAAAQAVQTAAQN | |
| | GVRAMSSLGSSLGSSGLGGGVAANLGRAASVGSLSVPQAWAAANQA | |
| | VTPAARALPLTSLTSAAERGPGQMLGGLPVGQMGARAGGGLSGVLR | |
| | VPPRPYVMPHSPAAG | |
| PE13 | VSFVMAYPEMLAAAADTLQSIGATTVASNAAAAAPTTGVVPPAADEVS | SEQ ID |
| (Rv1195) | ALTAAHFAAHAAMYQSVSARAAAIHDQFVATLASSASSYAATEVANAA | NO: 4 |
| 9.58 kDa | AAS | |

TABLE 2
Codon optimised and uridine depleted nucleic acid sequences encoding
the PE13, CFP-10, WbbL1, and PPE18 proteins.

| M.tb | | |
|---|---|---|
| antigenic | | SEQ ID |
| protein | Sequence** | NO |
| Wbbl1 | GTGGTGGCCGTGACCTACAGCCCCGGCCCCCACCTGGAGAGATTC | SEQ ID |
| (Rv3265c) | CTGGCTAGCCTGAGCCTGGCCACCGAAAGACCCGTGTCCGTGCTT | NO: 5 |
| 14.5% T | TTGGCCGACAACGGCAGTACGGACGGCACCCCCCAAGCCGCCGT | |
| | GCAGAGATACCCCAACGTGAGACTGCTGCCCACCGGCGCCAACCT | |
| | GGGCTACGGCACCGCCGTGAATAGAACGATAGCTCAGCTGGGCGA | |
| | GATGGCCGGAGACGCCGGTGAACCCTGGGTGGACGATTGGGTCAT | |
| | AGTGGCGAATCCCGACGTGCAATGGGGTCCCGGCAGCATCGATGC | |
| | CCTGCTGGATGCCGCTAGCAGATGGCCCCGCGCTGGTGCCCTGG | |
| | GACCGTTAATTCGGGACCCCGACGGCAGCGTGTACCCTAGCGCTA | |
| | GACAGATGCCTAGCCTGATCAGAGGCGGCATGCACGCCGTGCTGG | |
| | GCCCCTTCTGGCCTAGAAACCCCTGGACCACCGCCTACAGACAAG | |
| | AGAGACTGGAGCCTAGCGAAAGACCCGTGGGCTGGCTGTCGGGCT | |
| | CCTGCCTGCTGGTGAGAAGAAGCGCCTTCGGCCAAGTGGGCGGCT | |
| | TCGACGAGAGATACTTCATGTACATGGAGGACGTGGACCTGGGCG | |
| | ACAGACTGGGCAAGGCCGGCTGGCTAAGCGTTTACGTGCCTAGCG | |
| | CCGAGGTGCTGCACCACAAGGCCCACAGCACCGGCCGGGACCCC | |
| | GCTAGCCACCTGGCCGCCCACCACAAGAGCACCTACATCTTCCTG | |
| | GCCGACAGACACAGCGGCTGGTGGAGAGCCCCCCTGAGATGGAC | |
| | CCTGAGAGGCAGCCTGGCCCTGAGAAGCCACCTGATGGTGAGAAG | |
| | CAGCCTGAGAAGAAGCAGAAGAAGAAAGCTGAAGCTGGTGGAGGG | |
| | CAGACAC | |
| CFP-10 | GCCGAGATGAAAACCGACGCCGCCACCCTGGCCCAAGAGGCCGG | SEQ ID |
| (Rv3874) | CAACTTCGAGAGAATCAGCGGCGACCTCAAGACACAGATCGACCA | NO: 6 |
| 9.67% T | AGTGGAGAGCACGGCTGGCAGCCTGCAAGGGCAATGGAGAGGCG | |
| | CAGCCGGTACTGCCGCACAAGCCGCCGTGGTGAGATTCCAAGAGG | |
| | CCGCCAACAAGCAGAAGCAAGAGCTGGACGAGATCAGCACCAACA | |
| | TCAGACAAGCCGGCGTGCAGTACAGCAGAGCCGACGAGGAGCAG | |
| | CAGCAAGCCCTGAGCTCTCAGATGGGCTTC | |
| PPE18 | GTGGACTTCGGCGCCCTGCCCCCCGAGATAAACTCCGCTAGAATG | SEQ ID |
| (Rv1196) | TACGCCGGCCCGGGAAGCGCTAGCTTGGTCGCAGCCGCACAGAT | NO: 7 |
| 15.14% T | GTGGGACAGCGTGGCTAGCGACCTGTTCAGCGCCGCTAGCGCCTT | |
| | TCAGTCGGTGGTGTGGGGACTTACTGTCGGCAGCTGGATCGGCAG | |
| | TTCGGCTGGCTTGATGGTGGCCGCGGCGTCGCCCTATGTGGCCTG | |
| | GATGAGCGTGACTGCCGGGCAAGCCGAACTGACTGCGGCTCAAGT | |
| | GCGTGTGGCCGCGGCTGCCTATGAAACCGCGTACGGCCTTACAGT | |
| | TCCGCCTCCCGTGATCGCCGAGAACAGAGCCGAGCTGATGATCCT | |
| | GATCGCCACCAACCTGCTGGGGCAGAACACCCCCGCCATCGCCGT | |
| | GAACGAGGCCGAGTACGGCGAGATGTGGGCTCAAGATGCTGCAGC | |
| | GATGTTCGGATACGCCGCCGCCACGGCGACCGCCACCGCCACCTT | |
| | ACTGCCCTTCGAAGAAGCCCCCGAGATGACAAGCGCCGGCGGCTT | |
| | ATTAGAGCAAGCCGCAGCCGTGGAGGAGGCTAGCGACACGGCCG | |
| | CTGCCAATCAGCTGATGAACAATGTGCCCCAAGCCCTACAACAACT | |
| | GGCGCAGCCCACCCAAGGCACAACCCCAAGCAGCAAGCTGGGCG | |
| | GCCTGTGGAAAACCGTGAGCCCCCACAGAAGCCCCATCAGCAACA | |
| | TGGTGAGCATGGCCAACAACCACATGAGCATGACAAACAGTGGCG | |
| | TGAGCATGACCAATACGCTCAGCAGCATGCTGAAGGGCTTCGCGC | |
| | CGGCCGCCGCTGCACAAGCTGTTCAGACTGCGGCGCAGAACGGG | |
| | GTGAGAGCAATGAGCAGCCTGGGCTCAAGCCTTGGCAGCTCTGGC | |
| | TTAGGCGGCGGTGTGGCAGCCAACCTGGGCAGAGCCGCCTCAGTT | |
| | GGGAGCCTGTCCGTGCCTCAAGCCTGGGCAGCGGCGAATCAAGCT | |
| | GTCACCCCCGCCGCTAGAGCCCTGCCCCTGACAAGCCTGACTAGT | |
| | GCGGCAGAGAGAGGCCCCGGCCAAATGCTCGGTGGCTTACCGGT | |
| | AGGGCAAATGGGGGCAAGAGCGGGAGGCGGTCTTTCCGGCGTGC | |
| | TGAGAGTGCCCCCTAGACCCTACGTGATGCCCCACAGCCCCGCCG | |
| | CCGGC | |
| PE13 | GTGTCTTTCGTGATGGCCTACCCCGAAATGCTAGCCGCCGCCGCA | SEQ ID |
| (Rv1195) | GATACCCTGCAGAGCATCGGCGCAACCACAGTTGCAAGCAATGCT | NO: 8 |
| 15% T | GCTGCTGCCGCACCGACTACCGGCGTGGTGCCCCCCGCGGCGGA | |
| | TGAGGTGAGCGCCCTAACCGCCGCCCATTTCGCCGCACACGCCGC | |
| | GATGTATCAGAGCGTGAGCGCTAGAGCCGCCGCCATCCACGATCA | |
| | GTTCGTGGCCACCCTGGCTAGCAGCGCTAGCAGCTACGCCGCCAC | |
| | CGAGGTGGCCAACGCGGCCGCCGCTAGC | |

*Any of the sequences in Table 1 may include a methionine residue at the start, particularly where the sequence is the first sequence in the construct.
**Any of the sequences in Table 2 may include a start codon (ATG) at the 5′ end, particularly where the sequence is the first sequence in the construct. Further, any of the sequences may include a stop codon selected from TAA, TAG, or TGA at the 3′ end, particularly where the sequence is the last sequence in the construct. In particular, the inventors of the present invention have designed a specific stop codon sequence, comprising four consecutive stop codons with the nucleotide sequence of TGATAATGATAG (SEQ ID NO: 32). Individual antigenic sequences may also include the stop sequence of SED ID NO: 32.

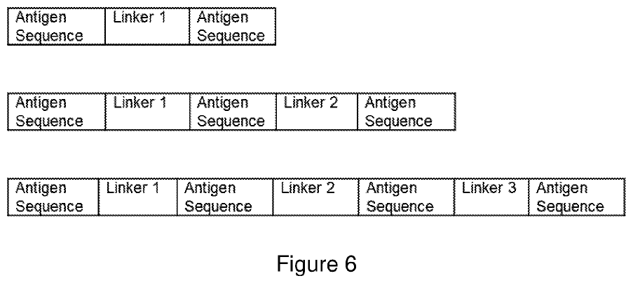

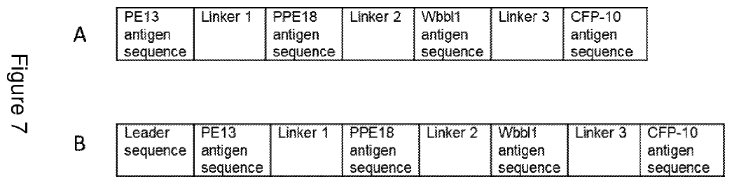

Various construct designs were employed, including wherein the polyprotein expressing sequence contains two or more of the codon optimised sequences encoding PE13, CFP-10, WbbL1, and PPE18 proteins in any order within the polyprotein-expressing sequence and linked by glycine-serine and/or glycine flexible linkers. In a further design, the polyprotein expressing sequence contains two or more of the codon optimised sequences encoding PE13, CFP-10, WbbL1, and PPE18 proteins in any order within the polyprotein-expressing sequence and linked by 2A-derived peptides. Alternatively, the polyprotein expressing sequence contains two or more of the codon optimised sequences encoding PE13, CFP-10, WbbL1, and PPE18 proteins in any order within the polyprotein-expressing sequence and linked by glycine-serine and/or glycine flexible linkers and 2A-derived peptides. The glycine-serine, glycine, and 2A-derived sequences investigated for incorporation in the vaccine constructs are provided in Table 3 below. FIG. 6 shows the various polyprotein constructs that were designed. The linkers in position 1, positions 1 and 2, and/or positions 1, 2 and 3 are derived from the sequences provided in Table 3, while the antigen sequences are selected from the codon optimised sequences encoding PE13, CFP-10, WbbL1, and PPE18 proteins in any order within the polyprotein-expressing sequence. The first and/or last four codons of each of the codon optimised sequences encoding PE13, CFP-10, WbbL1, and PPE18 proteins may be modified or removed to facilitate the inclusion of the linker.

TABLE 3
Glycine-serine, glycine and 2A-derived linker sequences investigated
in the polyprotein construct design.

| Linker sequence | SEQ ID NO |
|---|---|
| GGCGGATCTGGCGGCAGC | SEQ ID NO: 9 |
| GGCGGCGGCAGCGGCGGCGGATCT | SEQ ID NO: 10 |
| GGCGGCGGCGGCAGCGGCGGAGGCGGATCT | SEQ ID NO: 11 |
| GGCGGAGGCGGATCTGGCGGAGGCGGCAGCGGCGGC | SEQ ID NO: 12 |
| GGCGGCAGC | |
| GGCGGCGGCGGCGGAGGCGGTGGC | SEQ ID NO: 13 |
| GGCGGCGGCGGCGGAGGC | SEQ ID NO: 14 |
| GGCGGCGGCGGC | SEQ ID NO: 15 |
| GGATCTGGCGAGGGCAGAGGCAGCCTGCTGACCTGCGG | SEQ ID NO: 16 |
| CGACGTGGAAGAGAACCCCGGCCCT | |
| GGATCTGGCGCCACCAACTTCAGCCTGCTGAAGCAGG | SEQ ID NO: 17 |
| CCGGCGACGTGGAAGAGAACCCCGGCCCT | |
| GGATCTGGCGTGAAGCAGACCCTGAACTTCGACCTGCT | SEQ ID NO: 18 |
| GAAGCTGGCCGGCGATGTGGAGAGCAACCCCGGCCCT | |
| GGATCTGGCCAGTGCACCAACTACGCCCTGCTGAAGCT | SEQ ID NO: 19 |
| GGCCGGCGACGTGGAGAGCAACCCCGGCCCT | |

Analysis of naturally occurring sequences encoding CFP-10, WbbL1, and PPE18 do not reveal mammalian secretory peptide signals (SPs) such that these proteins would remain in the cytosol. Thus, to enhance antigen secretion, sequences encoding SPs with established efficiency were incorporated into the mRNA template plasmids. These were typically positioned upstream and in-frame of the M.tb antigen ORFs (i.e., at the N-terminal ends of the proteins). The immunogenicity of constructs with the following leader sequences were investigated:
leader sequence derived from Mycobacterium tuberculosis Rv2878c sequence
leader sequence derived from Mycobacterium tuberculosis MT18B_2507 sequence
leader sequence derived from Mycobacterium tuberculosis Rv3803c sequence
leader sequence derived from Mycobacterium tuberculosis Rv1860 sequence
leader sequence derived from the human interleukin 2 sequence
leader sequence derived from Gaussia luciferase

The leader sequences investigated for incorporation in the vaccine constructs are provided in Table 4 below, together with the codon optimised sequences encoding the leader peptides. The leader sequence is included either at the N-terminal of the individual antigen sequences or at the N-terminal of the polyprotein including two or more antigen sequences, where the antigen sequences are selected from the codon optimised sequences encoding PE13, CFP-10, WbbL1, and PPE18 proteins in any order within the polyprotein-expressing sequence. The first and/or last four codons of each of the codon optimised sequences encoding PE13, CFP-10, WbbL1, and PPE18 proteins may be modified or removed to facilitate the inclusion of the leader sequence.

TABLE 4
Leader sequences investigated in the polyprotein construct design and
the codon optimised sequences encoding the leader sequences.

| Source | Sequence type | Sequence | SEQ ID NO |
|---|---|---|---|
| Mycobacterium | Amino acid | MSLRLVSPIKAFADGIVAVAIAVVLMFGLANTPRAVA | SEQ ID |
| tuberculosis | | | NO: 20 |
| Rv2878c | Codon-optimised | ATGAGCCTGCGGCTGGTGTCTCCCATCAAGGCCTTCGCCGACGGCATCGT | SEQ ID |
| | nucleotide | GGCCGTGGCTATCGCCGTGGTGCTGATGTTCGGCCTGGCCAACACCCCTA | NO: 21 |
| | | GAGCCGTCGCT | |
| Mycobacterium | Amino acid | MKLTTMIKTAVAVVAMAAIATFAAPVALA | SEQ ID |
| tuberculosis | | | NO: 22 |
| MT18B 2507 | Codon-optimised | ATGAAGCTGACCACCATGATCAAGACCGCCGTGGCTGTGGTCGCCATGG | SEQ ID |
| | nucleotide | CCGCTATCGCCACATTCGCCGCCCCTGTGGCCCTGGCC | NO: 23 |
| Mycobacterium | Amino acid | MKGRSALLRALWIAALSFGLGGVAVA | SEQ ID |
| tuberculosis | | | NO: 24 |
| Rv3803c | Codon-optimised | ATGAAGGGCAGATCTGCCCTGCTGCGGGCCCTGTGGATC | SEQ ID |
| | nucleotide | GCCGCTCTGAGCTTCGGCCTGGGCGGAGTGGCCGTGGCC | NO: 25 |
| Mycobacterium | Amino acid | MHQVDPNLTRRKGRLAALAIAAMASASLVTVAVPATANA | SEQ ID |
| tuberculosis | | | NO: 26 |
| Rv1860 | Codon-optimised | ATGCACCAGGTCGACCCCAACCTGACGAGACGGAAGGGCAGACTGGGGG | SEQ ID |
| | nucleotide | CCGTGGCCATCGCCGCTATGGCCAGCGCCTCTCTGGTGACCGTGGCGGT | NO: 27 |
| | | GCCTGCTACAGCCAACGCC | |
| Human | Amino acid | MYRMQLLSCIALSLALVTNS | SEQ ID |
| interleukin 2 | | | NO: 28 |
| | Codon-optimised | ATGTACAGAATGCAGCTGCTGAGCTGCATCGCCCTGAGCCTGGCCCTGG | SEQ ID |
| | nucleotide | TGACCAACAGC | NO: 29 |

### Cloning and Propagation of Plasmids Encoding M. tb Proteins

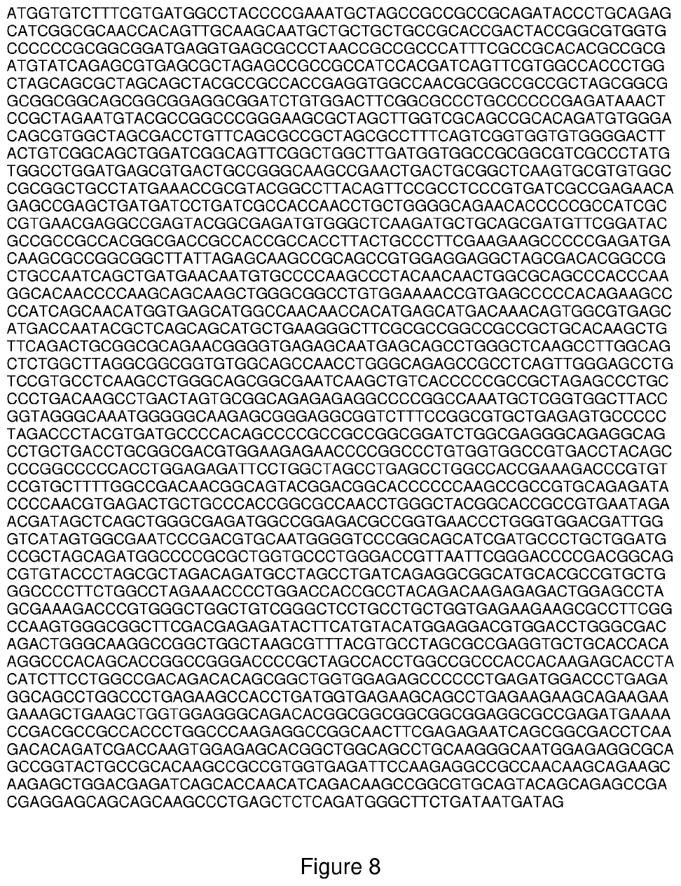

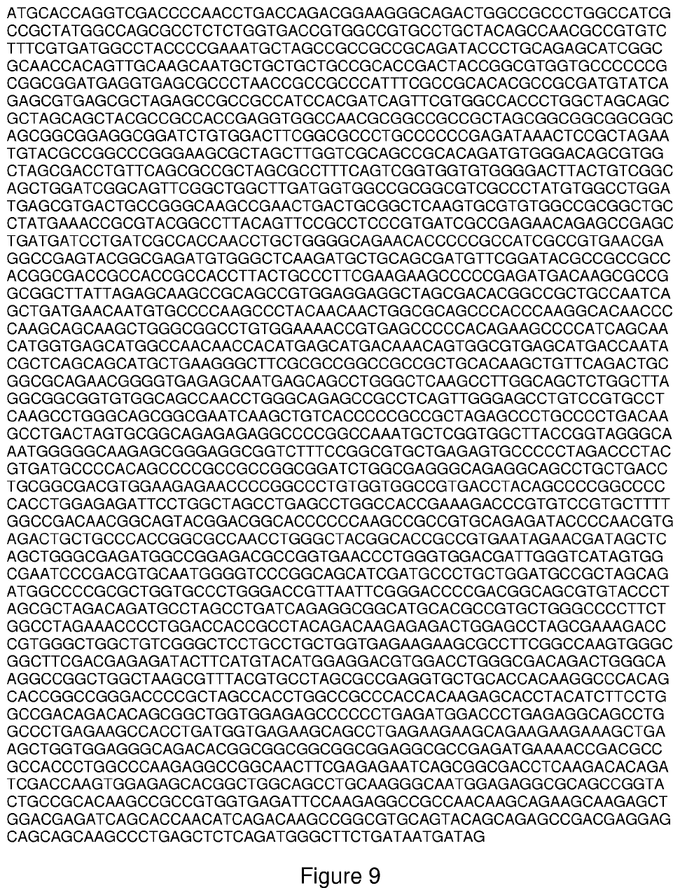

DNA sequences encoding individual M. tb antigens (i.e., WbbL1, PE13, PPE18, and CFP-10) as well as combinations in polyproteins were generated using gene synthesis as described above. The antigen-encoding sequences were sub-cloned into a T7 RNA polymerase-based vector to facilitate in vitro transcription of these sequences as eukaryotic mRNA-like transcripts. Leader sequences were generated by oligonucleotide extension and polymerase chain reaction (PCR), or by gene synthesis, and cloned upstream of the individual M. tb antigen or polyproteins. Two exemplary constructs are provided in FIG. 7. The sequences of these two constructs correspond to SEQ ID NO:30 and SEQ ID NO:31, respectively. The construct provided in FIG. 7A does not include a leader sequence, while the construct provided in FIG. 7B includes a leader sequence of SEQ ID NO:24 encoded by the codon optimised sequence of SEQ ID NO:25. In both constructs “linker 1” is a linker of SEQ ID NO: 11, “linker 2” is a linker of SEQ ID NO: 16, and “linker 3” is a linker of SEQ ID NO: 14. The nucleic acid sequences of these exemplary constructs are further provided in FIG. 8 and FIG. 9, where the sequence in FIG. 8 corresponds to the construct shown in FIG. 7A without the leader sequence and the sequence in FIG. 9 corresponds to the construct shown in FIG. 7B including the leader sequence. Plasmids were propagated in bacteria and purified using anion-exchange resin columns.

### Example 4

Preparation of the Monogenic M.tb mRNA Vaccines
Synthesis of mRNA

In vitro transcription (IVT) was carried out according to established methods employing restriction-linearised plasmid DNA and then IVT. Co-transcriptional capping (e.g., with CleanCap substrates from TriLink) or post transcriptionally with the vaccinia capping enzyme were employed. Modified nucleotides (e.g., N1-methyl-pseudouridine and pseudouridine) were included in the IVT reactions. This is intended to diminish innate immune responses to the synthesised mRNA candidates. In the present example, linearised plasmids encoding the four monogenic M. tb antigens, CFP-10, WbbL1, PE-13 and PPE-18, with or without leader sequences were used as templates for mRNA chemical synthesis. IVT reactions were carried out using the bacteriophage T7 polymerase in the presence of CleanCapCap® Reagent AG (TriLink BioTechnologies) and pseudouridine.

Purification and Analysis of In Vitro Transcribed mRNA

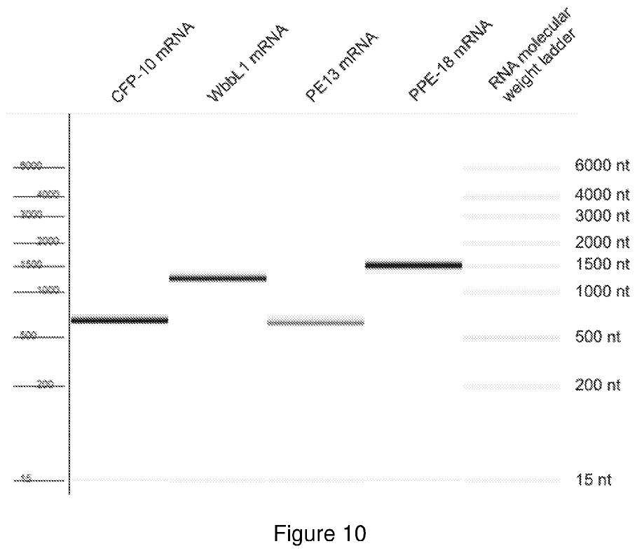

Purification of mRNA for evaluation in cultured cells and in vivo was undertaken according to established methods including lithium chloride precipitation and Oligo (dT) column chromatography. Size and integrity of in vitro transcribed mRNA was assessed using capillary gel electrophoresis on the 5200 Fragment Analyzer using the DNF-471 RNA kit (15 nt) (Agilent). As shown in FIG. 10, in vitro transcribed mRNAs were successfully obtained for all four of the M.tb antigens, CFP-10, WbbL1, PE-13 and PPE-18.

In Vitro Characterisation of mRNA

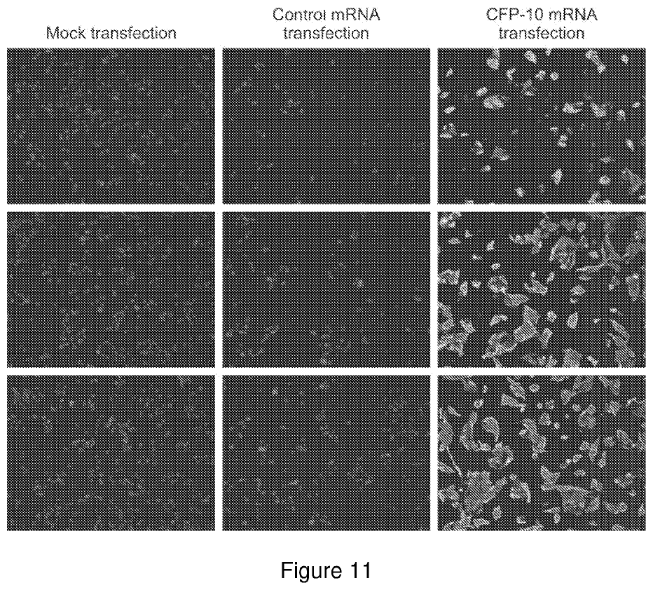

Immunofluorescence staining of M.tb antigens expressed from mRNA was assessed in cultured mammalian cells using established methods. Briefly, HepG2-hNTCP cells were seeded in a 96-well plate at approximately 15 000 cells/well (DMEM/F12 supplemented with GlutaMax™, 10% FBS, 10 mM HEPES, 50 UM hydrocortisone, 200 units/mL penicillin, 200 μg/mL streptomycin, 5 μg/mL insulin, and 400 μg/mL G418). The following day cells were transfected with 100 ng of CFP-10 encoding mRNA using Lipofectamine™ MessengerMAX™ (Thermo Fisher Scientific) and incubated at 37° C. and 5% CO₂. Twenty-four hours post-transfection, growth media was removed, and the cells were fixed with 4% PFA and permeabilised. Cells were stained with a rabbit polyclonal anti-CFP-10 primary antibody (Thermo Fisher Scientific, PA5-122324) and an Alexa-Fluor™ 488-conjugated goat anti-rabbit secondary antibody (Thermo Fisher Scientific, A-11008). The cells were counterstained with DAPI and imaged using the EVOS FL (Thermo Fisher Scientific) under the DAPI and GFP filters. FIG. 11 provides confirmation of the in situ expression of CFP-10 protein from the CFP-10 mRNA.

### Formulation of Lipid Nanoparticles (LNPs)

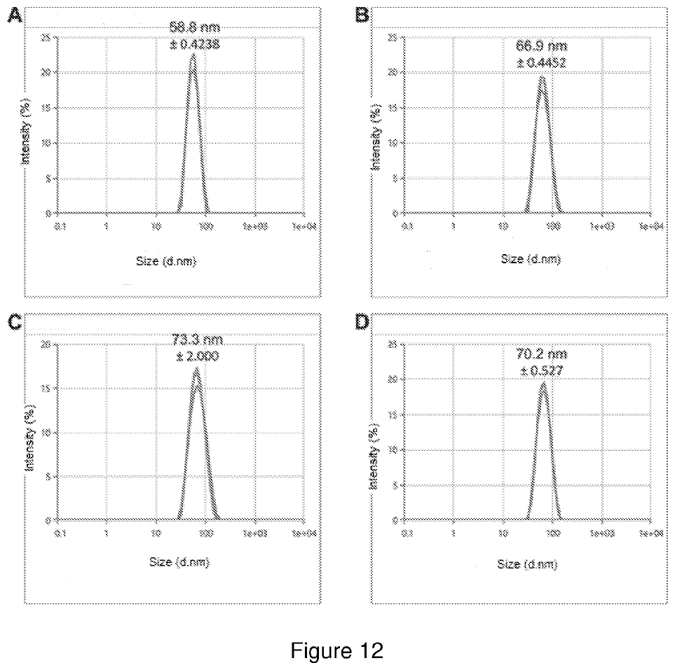

mRNA is incorporated into LNPs using proven methods of microfluidics and diafiltration or tangential flow filtration. Component lipids for the formulations include previously described ionisable lipids as well as novel renewable lipids. In the present example, TB-antigen encoding mRNAs were formulated as lipid nanoparticles (LNPs) with a helper lipid (DSPC), a pegylated lipid (PEG2000-DMG), cholesterol and the ionisable lipid SM102 using microfluidics-based assembly (NanoAssemblr® Ignite™, Precision Nanosystems). Characterisation using surface charge analysis, particle size determination and polydispersity index was carried out using the Zetasizer Pro (Malvern) (FIG. 12). LNPs for the mRNA antigens ranged from 58 nm-75.5 nm in size for monogenic mRNA.

### Example 5

### Induction of Cellular Immune Responses to Monogenic M. tb Antigen Vaccines

### Immunization of Mice and Preparation of Splenocytes

All animal work was carried out in accordance with protocols approved by the University of the Witwatersrand Animal Ethics Screening Committee. On day zero (DO), female BALB/c, C57BL6 and C3HeB/FeJ mice were injected intramuscularly with lipid-nanoparticle formulated TB antigen-expressing mRNA vaccines, including mRNAs encoding each of the four M. tb antigens, CFP-10, WbbL1, PE-13 and PPE-18 (10 μg/mouse in a total volume of 50 μl per vaccination), as well as a vaccine mix comprising equal ratios of all four monogenic mRNAs which were mixed prior to vaccinating mice (2.5 μg of each monogenic mRNA/mouse in a total volume of 50 μl per vaccination). Control groups were injected intramuscularly with saline (50 μl/mouse per vaccination) or a lipid-nanoparticle formulated control mRNA encoding Firefly Luciferase (10 μg/mouse in a total volume of 50 μl per vaccination). Mice received boost vaccinations three weeks later. Serum was collected directly before vaccinations (day zero and week 3) and at week 5. Spleens were collected at week 5 for analysis of antigen-specific T cells. Table 5 below shows that Animal groups used for the monogenic mRNA vaccines and the vaccine doses administered.

TABLE 5
Animal groups and doses for monogenic TB mRNA vaccines.

| | | | | Vaccine dose | |
|---|---|---|---|---|---|
| | | No. of | | (per dose) | |
| Group | Strain | mice | Vaccine | 2-dose series | |
| 1 | BALB/c | 3 | Saline | 50 | μl |
| 2 | BALB/c | 3 | Control mRNA | 10 | μg |
| 3 | BALB/c | 3 | CFP-10 mRNA | 10 | μg |
| 4 | BALB/c | 3 | WbbL1 mRNA | 10 | μg |
| 5 | BALB/c | 3 | PE-13 mRNA | 10 | μg |
| 6 | BALB/c | 3 | PPE-18 mRNA | 10 | μg |
| 7 | BALB/c | 3 | Mix (CWPP) | 10 μg (2.5 μg per | |
| | | | mRNA | antigen per dose) | |
| 8 | C57BL/6 | 3 | Saline | 50 | μl |
| 9 | C57BL/6 | 2 | Control mRNA | 10 | μg |
| 10 | C57BL/6 | 3 | CFP-10 mRNA | 10 | μg |
| 11 | C57BL/6 | 2 | WbbL1 mRNA | 10 | μg |
| 12 | C57BL/6 | 3 | PE-13 mRNA | 10 | μg |
| 13 | C57BL/6 | 3 | PPE-18 mRNA | 10 | μg |
| 14 | C57BL/6 | 2 | Mix (CWPP) | 10 μg (2.5 μg per | |
| | | | mRNA | antigen per dose) | |
| 15 | C3HeB/FeJ | 3 | Saline | 50 | μl |
| 16 | C3HeB/FeJ | 4 | CFP-10 mRNA | 10 | μg |
| 17 | C3HeB/FeJ | 3 | Mix (CWPP) | 10 μg (2.5 μg per | |
| | | | mRNA | antigen per dose) | |
| 18 | C3HeB/FeJ | 2 | L1-CFP-10 mRNA | 10 | μg |
| 19 | C3HeB/FeJ | 2 | L2-CFP-10 mRNA | 10 | μg |
| 20 | C3HeB/FeJ | 2 | L3-CFP-10 mRNA | 10 | μg |
| 21 | C3HeB/FeJ | 2 | L4-CFP-10 mRNA | 10 | μg |

Splenocytes were isolated from mice according to standard protocols. Briefly, spleens were mashed through 70 μm cell strainers and washed in PBS pH 7.2 supplemented with 1% FBS. Red blood cells were lysed using ammonium-chloride-potassium buffer. Cells were washed and resuspended in complete RPMI 1640 media supplemented with GlutaMax (Gibco), 10% FBS and antibiotics. Cells were seeded in 96-well U-bottom plates at a density of 1×10⁶ cells per well for ex vivo stimulations.

### T Cell Stimulation and Quantification of Secreted Cytokines

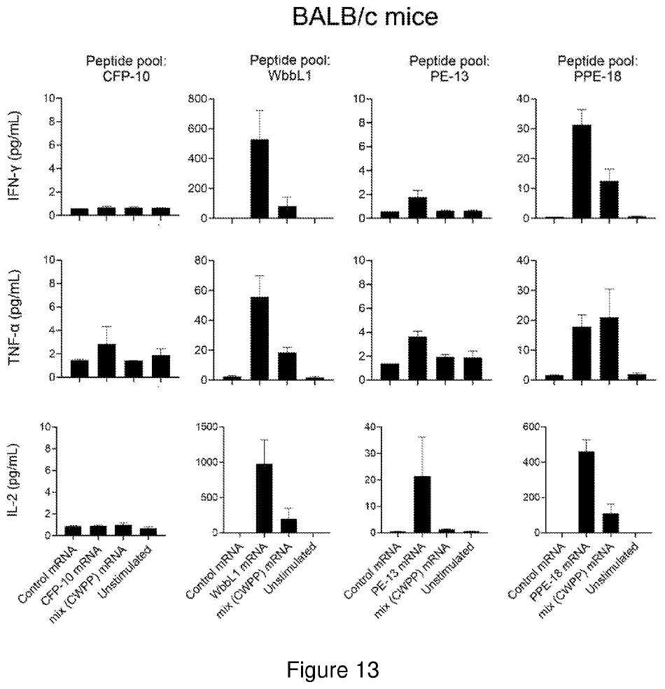

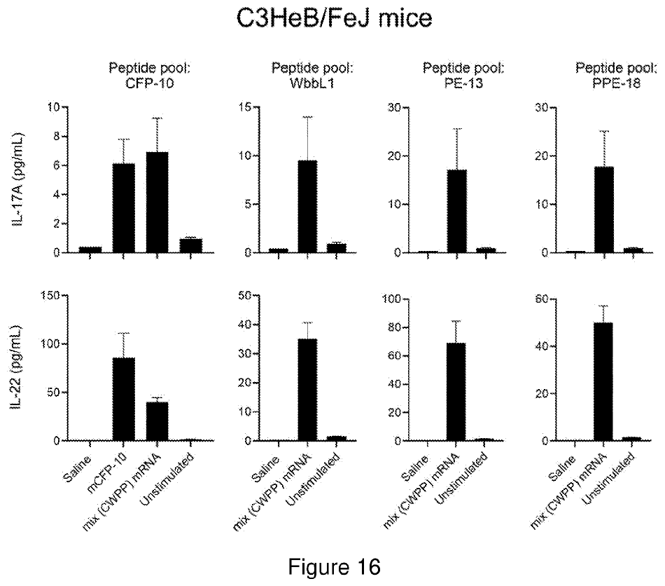

Peptide pools covering the M.tb mRNA vaccine antigens CFP-10, WbbL1, PE-13 and PPE-18, as well as M.tb non-vaccine antigens TB10.4, were synthesised by GenScript. Cells isolated from mouse spleens were stimulated with a cell stimulation cocktail (PMA and lonomycin) or 2 μg/ml peptide pools in the presence of 1 μg/ml anti-CD28 (clone 37.51) and incubated at 37° C. and 5% CO₂ for 24 hours. The concentration of secreted cytokines in the cell supernatants were quantified using the LEGENDplex™ MU Th1/Th2 Panel V03 (8-plex) (BioLegend), where the 8-plex panel detects IL-5, IL-13, IL-2, IL-6, IL-10, IFN-γ, TNF-α, IL-4, and the 12-plex panel detects IL-2, IL-4, IL-5, IL-6, IL-9, IL-10, IL-13, IL-17A, IL-17F, IL-22, IFN-γ and TNF-α. IFN-γ, TNF-α and IL-2 cytokine secretion profiles in ex vivo-stimulated spleen cells from immunised BALB/c, C57BI/6 and C3HeB/FeJ mice, respectively, are provided in FIGS. 13 to 15. IL-17A and IL-22 cytokine secretion profiles for C3HeB/FeJ mice are shown in FIG. 16.

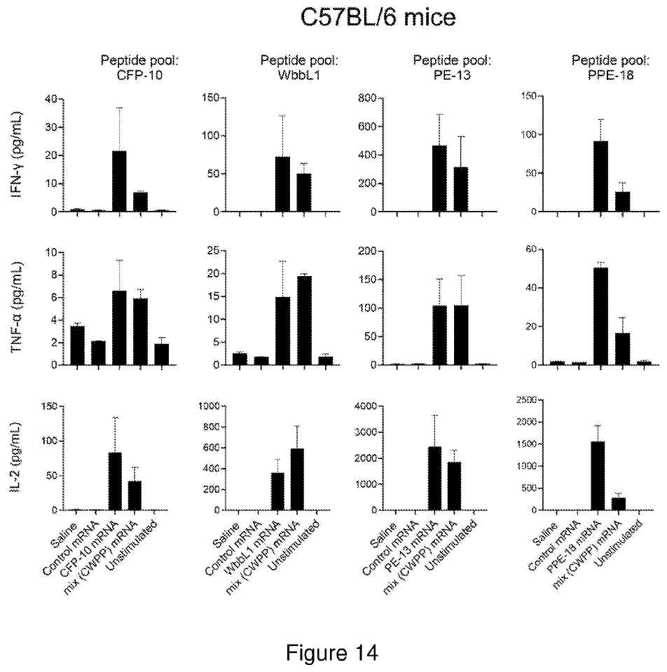

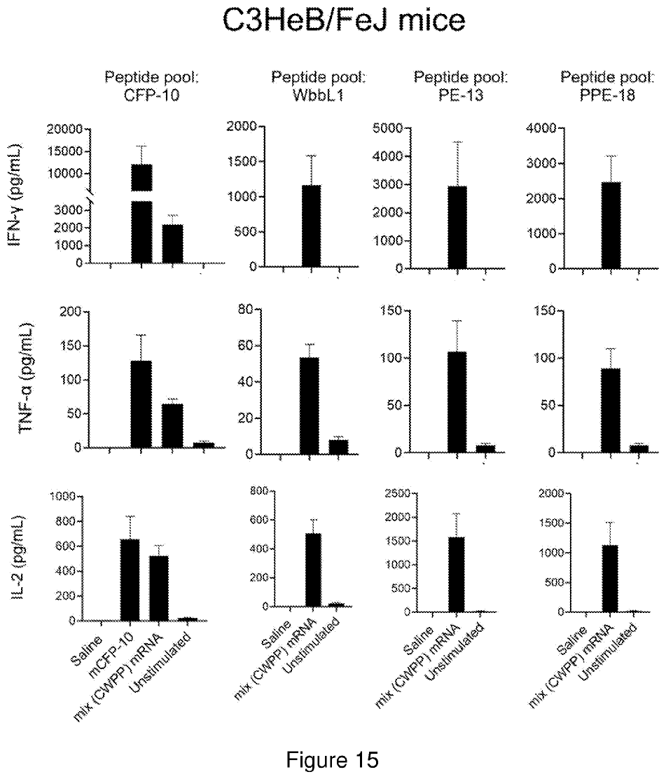

As can be seen from FIGS. 13 to 16, antigen-specific responses differ between mouse strains, as expected, due to differing haplotypes. From FIG. 13, increased concentrations of IFN-γ, TNF-α and IL-2 in BALB/c mice immunized with WbbL1, PPE-18, and mix (CWPP) mRNA were observed. Further, increased IL-2 was detected in mice immunized with PE-13 mRNA. From FIG. 14, increased concentrations of IFN-γ, TNF-α and IL-2 in C57BL/6 mice immunized with CFP-10, WbbL1, PE-13, PPE-18, and mix (CWPP) mRNA were observed. From FIG. 15, increased concentrations of IFN-γ, TNF-α and IL-2 in C3HeB/FeJ mice immunized with CFP-10 and mix (CWPP) mRNA were detected. Finally, increased concentrations of IL-17A and IL-22, indicative of Th17 response in vaccinated C3HeB/FeJ mice, are shown in FIG. 16.

### T Cell Stimulation and Intracellular Cytokine Staining

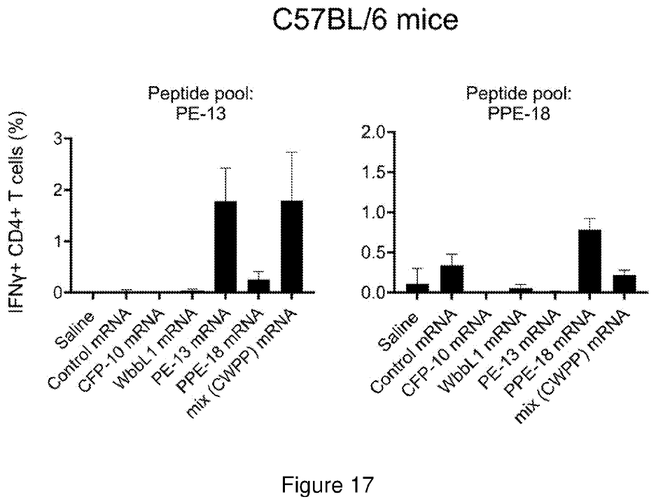

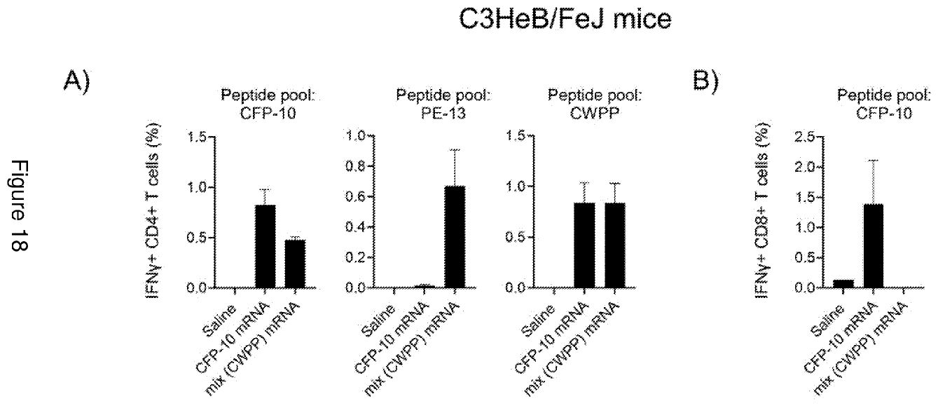

IFN-γ positive CD3, CD4 and CD8 T-cells were further detected using flow cytometry. Cell stimulations and intracellular cytokine staining was performed according to standard protocols. Briefly, cells were stimulated with a cell stimulation cocktail (PMA and lonomycin) or 2 μg/ml peptide pools in the presence of 1 μg/ml anti-CD28 (clone 37.51) for 1 hour at 37° C. and 5% CO₂, followed by the addition of Brefeldin A, and further incubation overnight (15 hours). Cells were washed, stained with a fixable viability dye, fixed and permeabilised using the BioLegend fixation buffer and intracellular staining perm wash buffer set. Cells were stained for intracellular IFN-γ-APC (clone XMG1.2), CD3-PerCP-eFluor™710 (clone 17A2) and CD4-FITC (clone GK1.5) or CD8-FITC (clone 53-67). Cell events were captured on a BD Accuri™ C6 plus flow cytometer (Beckton Dickinson) and FlowJo™ software (BD Life Sciences) was used to determine percentage of intracellular IFN-γ staining. Antigen-specific activation of CD4+ T cells was confirmed in vaccinated C57BL/6 mice (FIG. 17). In C3HeB/FeJ mice, CD4 and CD8 IFN-γ positive T cells were detected for the CFP-10 mRNA vaccine at a 10 μg dose and at a 2.5 μg dose which was included in the mix (CWPP) mRNA vaccine (FIG. 18). Secreted cytokine profiles indicate that vaccination induced increases in M.tb antigen-specific T-helper type 1 (Th1) immune responses (increased concentrations of IFN-γ, TNF-α and IL-2) in all three strains of vaccinated mice. A polyantigenic Th1 response was also detected in C57BI/6 and C3HeB/FeJ mice immunized with the vaccine mix comprising all four M.tb antigens. Increased concentrations of IL-17A and IL-22 in C3HeB/FeJ vaccinated mice are indicative of a Th17 response, which aids in the control of initial TB infection. Intracellular cytokine staining shows an increase in the percentage of IFN-γ positive CD4 T cells (CD3⁺CD4⁺IFN-γ⁺) in C57BI/6 mice immunized with mRNA encoding the PE-13, PPE-18 and mix (CWPP) antigens. Intracellular cytokine staining shows an increase in the percentage of IFN-γ positive CD4 and CD8 T cells (CD3⁺CD8⁺IFN-γ⁺) in C3HeB/FeJ mice immunized with mRNA encoding the CFP-10 antigen. Furthermore, a polyantigenic increase in the percentage of IFN-γ positive CD4 T cells is shown in C3HeB/FeJ mice vaccinated with the mix (CWPP) mRNA vaccine.

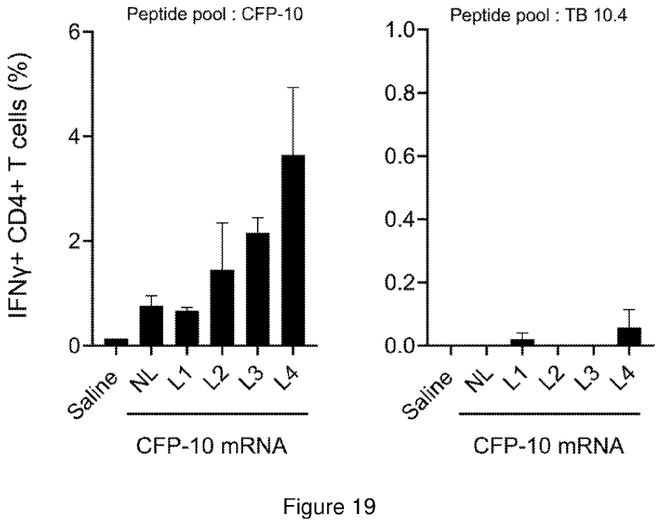

M. tb derived leader sequences L1 (SEQ ID NO:21), L2 (SEQ ID NO:23), L3 (SEQ ID NO: 25), and L4 (SEQ ID NO:27) were included in CFP-10 encoding mRNA vaccines. Increases in the percentage of IFN-γ +CD4+ T cells was observed in immunised C3HeB/FeJ mice when leader sequences 2, 3 and 4 were incorporated versus CFP-10 mRNA vaccines with no leader (NL) and L1-CFP-10 (FIG. 19).

These results thus provide evidence that the monogenic mRNA constructs are capable of eliciting antigen-specific T cell responses to the four M.tb antigens and that the mRNA vaccine are immunogenic in mice.

### Example 6

Preparation of Polygenic M.tb mRNA Vaccines
Synthesis of mRNA

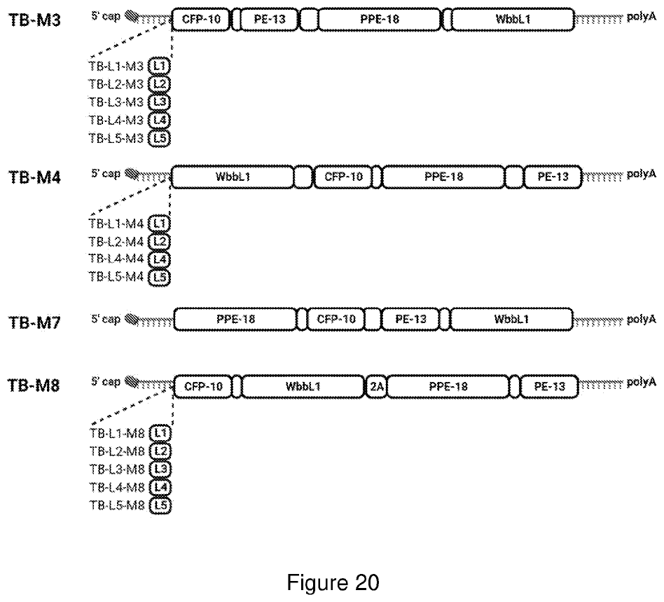

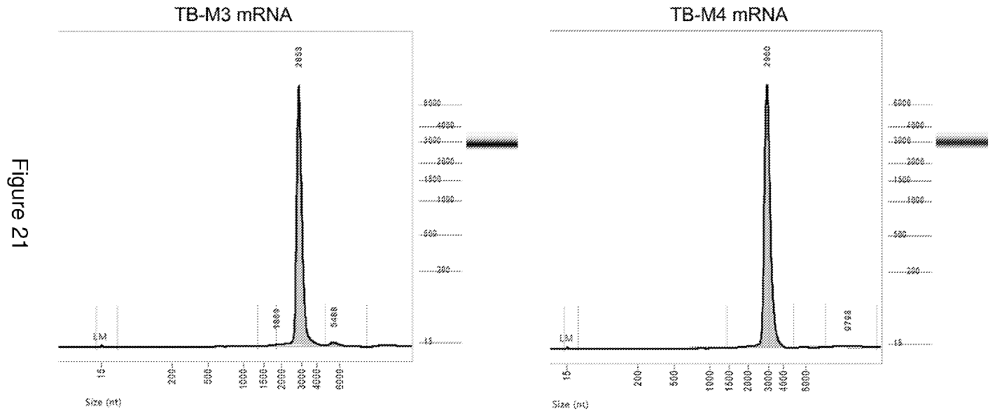

Various polyprotein mRNA vaccine constructs were developed, synthesised and purified using the same methods as described above. The polygenic constructs included M. tb antigens CFP-10, WbbL1, PE-13 and PPE-18 in different positions with and without leader sequences (L1, L2, L3, L4, L5) and are depicted in FIG. 20. The full amino acid sequences for the four polyproteins designated as TB-M3, TB-M4, TB-M7 and TB-M8 are provided as SEQ ID NOs: 33 to 36 as set out in Table 6 below. The full nucleic acid sequences of the polygenic constructs are provided as SEQ ID NOs: 37 to 40 as set out in Table 7 below. Further, any of the sequences in Table 6 may include one or more leader sequence selected from the leader sequences of SEQ ID NOs: 20, 22, 24, 26 and 28 and any of the sequences in Table 7 may include one or more leader sequence selected from the leader sequences of SEQ ID NOs: 21, 23, 25, 27 and 29 as described in Table 4 above. Plasmids were propagated in bacteria and purified using anion-exchange resin columns.

TABLE 6
Amino acid sequences of the TB-M3, TB-M4, TB-M7, TB-M8 proteins.

| M.tb | | |
|---|---|---|
| antigenic | | SEQ ID |
| protein | Amino acid sequence* | NO |
| TB-M3 | AEMKTDAATLAQEAGNFERISGDLKTQIDQVESTAGSLQGQWRGAAG | SEQ ID |
| | TAAQAAVVRFQEAANKQKQELDEISTNIRQAGVQYSRADEEQQQALS | NO: 33 |
| | SQMGFGGGGVSFVMAYPEMLAAAADTLQSIGATTVASNAAAAAPTTG | |
| | VVPPAADEVSALTAAHFAAHAAMYQSVSARAAAIHDQFVATLASSASS | |
| | YAATEVANAAAASGGGGSGGGGSGGGGSVDFGALPPEINSARMYAG | |
| | PGSASLVAAAQMWDSVASDLFSAASAFQSVVWGLTVGSWIGSSAGL | |
| | MVAAASPYVAWMSVTAGQAELTAAQVRVAAAAYETAYGLTVPPPVIA | |
| | ENRAELMILIATNLLGQNTPAIAVNEAEYGEMWAQDAAAMFGYAAATA | |
| | TATATLLPFEEAPEMTSAGGLLEQAAAVEEASDTAAANQLMNNVPQAL | |
| | QQLAQPTQGTTPSSKLGGLWKTVSPHRSPISNMVSMANNHMSMTNS | |
| | GVSMTNTLSSMLKGFAPAAAAQAVQTAAQNGVRAMSSLGSSLGSSGL | |
| | GGGVAANLGRAASVGSLSVPQAWAAANQAVTPAARALPLTSLTSAAE | |
| | RGPGQMLGGLPVGQMGARAGGGLSGVLRVPPRPYVMPHSPAAGGG | |
| | GGVVAVTYSPGPHLERFLASLSLATERPVSVLLADNGSTDGTPQAAVQ | |
| | RYPNVRLLPTGANLGYGTAVNRTIAQLGEMAGDAGEPWVDDWVIVAN | |
| | PDVQWGPGSIDALLDAASRWPRAGALGPLIRDPDGSVYPSARQMPSL | |
| | IRGGMHAVLGPFWPRNPWTTAYRQERLEPSERPVGWLSGSCLLVRR | |
| | SAFGQVGGFDERYFMYMEDVDLGDRLGKAGWLSVYVPSAEVLHHKA | |
| | HSTGRDPASHLAAHHKSTYIFLADRHSGWWRAPLRWTLRGSLALRSH | |
| | LMVRSSLRRSRRRKLKLVEGRH | |
| TB-M4 | VVAVTYSPGPHLERFLASLSLATERPVSVLLADNGSTDGTPQAAVQRY | SEQ ID |
| | PNVRLLPTGANLGYGTAVNRTIAQLGEMAGDAGEPWVDDWVIVANPD | NO: 34 |
| | VQWGPGSIDALLDAASRWPRAGALGPLIRDPDGSVYPSARQMPSLIR | |
| | GGMHAVLGPFWPRNPWTTAYRQERLEPSERPVGWLSGSCLLVRRSA | |
| | FGQVGGFDERYFMYMEDVDLGDRLGKAGWLSVYVPSAEVLHHKAHS | |
| | TGRDPASHLAAHHKSTYIFLADRHSGWWRAPLRWTLRGSLALRSHLM | |
| | VRSSLRRSRRRKLKLVEGRHGGGGSGGGGSGGGGSAEMKTDAATLA | |
| | QEAGNFERISGDLKTQIDQVESTAGSLQGQWRGAAGTAAQAAVVRFQ | |
| | EAANKQKQELDEISTNIRQAGVQYSRADEEQQQALSSQMGFGGGGV | |
| | DFGALPPEINSARMYAGPGSASLVAAAQMWDSVASDLFSAASAFQSV | |
| | VWGLTVGSWIGSSAGLMVAAASPYVAWMSVTAGQAELTAAQVRVAA | |
| | AAYETAYGLTVPPPVIAENRAELMILIATNLLGQNTPAIAVNEAEYGEM | |
| | WAQDAAAMFGYAAATATATATLLPFEEAPEMTSAGGLLEQAAAVEEA | |
| | SDTAAANQLMNNVPQALQQLAQPTQGTTPSSKLGGLWKTVSPHRSPI | |
| | SNMVSMANNHMSMTNSGVSMTNTLSSMLKGFAPAAAAQAVQTAAQN | |
| | GVRAMSSLGSSLGSSGLGGGVAANLGRAASVGSLSVPQAWAAANQA | |
| | VTPAARALPLTSLTSAAERGPGQMLGGLPVGQMGARAGGGLSGVLR | |
| | VPPRPYVMPHSPAAGGGGGSGGGGSGGGGSVSFVMAYPEMLAAAA | |
| | DTLQSIGATTVASNAAAAAPTTGVVPPAADEVSALTAAHFAAHAAMYQ | |
| | SVSARAAAIHDQFVATLASSASSYAATEVANAAAAS | |
| TB-M7 | VDFGALPPEINSARMYAGPGSASLVAAAQMWDSVASDLFSAASAFQS | SEQ ID |
| | VVWGLTVGSWIGSSAGLMVAAASPYVAWMSVTAGQAELTAAQVRVA | NO: 35 |
| | AAAYETAYGLTVPPPVIAENRAELMILIATNLLGQNTPAIAVNEAEYGEM | |
| | WAQDAAAMFGYAAATATATATLLPFEEAPEMTSAGGLLEQAAAVEEA | |
| | SDTAAANQLMNNVPQALQQLAQPTQGTTPSSKLGGLWKTVSPHRSPI | |
| | SNMVSMANNHMSMTNSGVSMTNTLSSMLKGFAPAAAAQAVQTAAQN | |
| | GVRAMSSLGSSLGSSGLGGGVAANLGRAASVGSLSVPQAWAAANQA | |
| | VTPAARALPLTSLTSAAERGPGQMLGGLPVGQMGARAGGGLSGVLR | |
| | VPPRPYVMPHSPAAGGGGGAEMKTDAATLAQEAGNFERISGDLKTQI | |
| | DQVESTAGSLQGQWRGAAGTAAQAAVVRFQEAANKQKQELDEISTNI | |
| | RQAGVQYSRADEEQQQALSSQMGFGGGGSGGGGSGGGGSVSFVM | |
| | AYPEMLAAAADTLQSIGATTVASNAAAAAPTTGVVPPAADEVSALTAA | |
| | HFAAHAAMYQSVSARAAAIHDQFVATLASSASSYAATEVANAAAASGG | |
| | GGVVAVTYSPGPHLERFLASLSLATERPVSVLLADNGSTDGTPQAAVQ | |
| | RYPNVRLLPTGANLGYGTAVNRTIAQLGEMAGDAGEPWVDDWVIVAN | |
| | PDVQWGPGSIDALLDAASRWPRAGALGPLIRDPDGSVYPSARQMPSL | |
| | IRGGMHAVLGPFWPRNPWTTAYRQERLEPSERPVGWLSGSCLLVRR | |
| | SAFGQVGGFDERYFMYMEDVDLGDRLGKAGWLSVYVPSAEVLHHKA | |
| | HSTGRDPASHLAAHHKSTYIFLADRHSGWWRAPLRWTLRGSLALRSH | |
| | LMVRSSLRRSRRRKLKLVEGRH | |
| TB-M8 | AEMKTDAATLAQEAGNFERISGDLKTQIDQVESTAGSLQGQWRGAAG | SEQ ID |
| | TAAQAAVVRFQEAANKQKQELDEISTNIRQAGVQYSRADEEQQQALS | NO: 36 |
| | SQMGFGGGGVVAVTYSPGPHLERFLASLSLATERPVSVLLADNGSTD | |
| | GTPQAAVQRYPNVRLLPTGANLGYGTAVNRTIAQLGEMAGDAGEPWV | |
| | DDWVIVANPDVQWGPGSIDALLDAASRWPRAGALGPLIRDPDGSVYP | |
| | SARQMPSLIRGGMHAVLGPFWPRNPWTTAYRQERLEPSERPVGWLS | |
| | GSCLLVRRSAFGQVGGFDERYFMYMEDVDLGDRLGKAGWLSVYVPS | |
| | AEVLHHKAHSTGRDPASHLAAHHKSTYIFLADRHSGWWRAPLRWTLR | |
| | GSLALRSHLMVRSSLRRSRRRKLKLVEGRHGSGATNFSLLKQAGDVE | |
| | ENPGPMVDFGALPPEINSARMYAGPGSASLVAAAQMWDSVASDLFSA | |
| | ASAFQSVVWGLTVGSWIGSSAGLMVAAASPYVAWMSVTAGQAELTA | |
| | AQVRVAAAAYETAYGLTVPPPVIAENRAELMILIATNLLGQNTPAIAVNE | |
| | AEYGEMWAQDAAAMFGYAAATATATATLLPFEEAPEMTSAGGLLEQA | |
| | AAVEEASDTAAANQLMNNVPQALQQLAQPTQGTTPSSKLGGLWKTVS | |
| | PHRSPISNMVSMANNHMSMTNSGVSMTNTLSSMLKGFAPAAAAQAV | |
| | QTAAQNGVRAMSSLGSSLGSSGLGGGVAANLGRAASVGSLSVPQAW | |
| | AAANQAVTPAARALPLTSLTSAAERGPGQMLGGLPVGQMGARAGGG | |
| | LSGVLRVPPRPYVMPHSPAAGGGGGVSFVMAYPEMLAAAADTLQSIG | |
| | ATTVASNAAAAAPTTGVVPPAADEVSALTAAHFAAHAAMYQSVSARAA | |
| | AIHDQFVATLASSASSYAATEVANAAAAS | |

TABLE 7
Codon optimised and uridine depleted nucleic acid sequences encoding
the TB-M3, TB-M4, TB-M7, TB-M8 proteins.

| M.tb | | |
|---|---|---|
| antigenic | | SEQ ID |
| protein | Sequence** | NO |
| TB-M3 | GCCGAGATGAAAACCGACGCCGCCACCCTGGCCCAAGAGGCCGG | SEQ ID |
| | CAACTTCGAGAGAATCAGCGGCGACCTCAAGACACAGATCGACCA | NO: 37 |
| | AGTGGAGAGCACGGCTGGCAGCCTGCAAGGGCAATGGAGAGGCG | |
| | CAGCCGGTACTGCCGCACAAGCCGCCGTGGTGAGATTCCAAGAGG | |
| | CCGCCAACAAGCAGAAGCAAGAGCTGGACGAGATCAGCACCAACA | |
| | TCAGACAAGCCGGCGTGCAGTACAGCAGAGCCGACGAGGAGCAG | |
| | CAGCAAGCCCTGAGCTCTCAGATGGGCTTCGGCGGCGGCGGCGT | |
| | GTCTTTCGTGATGGCCTACCCCGAAATGCTAGCCGCCGCCGCAGA | |
| | TACCCTGCAGAGCATCGGCGCAACCACAGTTGCAAGCAATGCTGC | |
| | TGCTGCCGCACCGACTACCGGCGTGGTGCCCCCCGCGGCGGATG | |
| | AGGTGAGCGCCCTAACCGCCGCCCATTTCGCCGCACACGCCGCGA | |
| | TGTATCAGAGCGTGAGCGCTAGAGCCGCCGCCATCCACGATCAGT | |
| | TCGTGGCCACCCTGGCTAGCAGCGCTAGCAGCTACGCCGCCACCG | |
| | AGGTGGCCAACGCGGCCGCCGCTAGCGGCGGAGGCGGATCTGGC | |
| | GGAGGCGGCAGCGGCGGCGGCGGCAGCGTGGACTTCGGCGCCC | |
| | TGCCCCCCGAGATAAACTCCGCTAGAATGTACGCCGGCCCGGGAA | |
| | GCGCTAGCTTGGTCGCAGCCGCACAGATGTGGGACAGCGTGGCTA | |
| | GCGACCTGTTCAGCGCCGCTAGCGCCTTTCAGTCGGTGGTGTGGG | |
| | GACTTACTGTCGGCAGCTGGATCGGCAGTTCGGCTGGCTTGATGG | |
| | TGGCCGCGGCGTCGCCCTATGTGGCCTGGATGAGCGTGACTGCC | |
| | GGGCAAGCCGAACTGACTGCGGCTCAAGTGCGTGTGGCCGCGGC | |
| | TGCCTATGAAACCGCGTACGGCCTTACAGTTCCGCCTCCCGTGATC | |
| | GCCGAGAACAGAGCCGAGCTGATGATCCTGATCGCCACCAACCTG | |
| | CTGGGGCAGAACACCCCCGCCATCGCCGTGAACGAGGCCGAGTA | |
| | CGGCGAGATGTGGGCTCAAGATGCTGCAGCGATGTTCGGATACGC | |
| | CGCCGCCACGGCGACCGCCACCGCCACCTTACTGCCCTTCGAAGA | |
| | AGCCCCCGAGATGACAAGCGCCGGCGGCTTATTAGAGCAAGCCGC | |
| | AGCCGTGGAGGAGGCTAGCGACACGGCCGCTGCCAATCAGCTGAT | |
| | GAACAATGTGCCCCAAGCCCTACAACAACTGGCGCAGCCCACCCA | |
| | AGGCACAACCCCAAGCAGCAAGCTGGGCGGCCTGTGGAAAACCGT | |
| | GAGCCCCCACAGAAGCCCCATCAGCAACATGGTGAGCATGGCCAA | |
| | CAACCACATGAGCATGACAAACAGTGGCGTGAGCATGACCAATACG | |
| | CTCAGCAGCATGCTGAAGGGCTTCGCGCCGGCCGCCGCTGCACAA | |
| | GCTGTTCAGACTGCGGCGCAGAACGGGGTGAGAGCAATGAGCAGC | |
| | CTGGGCTCAAGCCTTGGCAGCTCTGGCTTAGGCGGCGGTGTGGCA | |
| | GCCAACCTGGGCAGAGCCGCCTCAGTTGGGAGCCTGTCCGTGCCT | |
| | CAAGCCTGGGCAGCGGCGAATCAAGCTGTCACCCCCGCCGCTAGA | |
| | GCCCTGCCCCTGACAAGCCTGACTAGTGCGGCAGAGAGAGGCCCC | |
| | GGCCAAATGCTCGGTGGCTTACCGGTAGGGCAAATGGGGGCAAGA | |
| | GCGGGAGGCGGTCTTTCCGGCGTGCTGAGAGTGCCCCCTAGACC | |
| | CTACGTGATGCCCCACAGCCCCGCCGCCGGCGGCGGCGGCGGCG | |
| | TGGTGGCCGTGACCTACAGCCCCGGCCCCCACCTGGAGAGATTCC | |
| | TGGCTAGCCTGAGCCTGGCCACCGAAAGACCCGTGTCCGTGCTTT | |
| | TGGCCGACAACGGCAGTACGGACGGCACCCCCCAAGCCGCCGTG | |
| | CAGAGATACCCCAACGTGAGACTGCTGCCCACCGGCGCCAACCTG | |
| | GGCTACGGCACCGCCGTGAATAGAACGATAGCTCAGCTGGGCGAG | |
| | ATGGCCGGAGACGCCGGTGAACCCTGGGTGGACGATTGGGTCATA | |
| | GTGGCGAATCCCGACGTGCAATGGGGTCCCGGCAGCATCGATGCC | |
| | CTGCTGGATGCCGCTAGCAGATGGCCCCGCGCTGGTGCCCTGGG | |
| | ACCGTTAATTCGGGACCCCGACGGCAGCGTGTACCCTAGCGCTAG | |
| | ACAGATGCCTAGCCTGATCAGAGGCGGCATGCACGCCGTGCTGGG | |
| | CCCCTTCTGGCCTAGAAACCCCTGGACCACCGCCTACAGACAAGA | |
| | GAGACTGGAGCCTAGCGAAAGACCCGTGGGCTGGCTGTCGGGCT | |
| | CCTGCCTGCTGGTGAGAAGAAGCGCCTTCGGCCAAGTGGGCGGCT | |
| | TCGACGAGAGATACTTCATGTACATGGAGGACGTGGACCTGGGCG | |
| | ACAGACTGGGCAAGGCCGGCTGGCTAAGCGTTTACGTGCCTAGCG | |
| | CCGAGGTGCTGCACCACAAGGCCCACAGCACCGGCCGGGACCCC | |
| | GCTAGCCACCTGGCCGCCCACCACAAGAGCACCTACATCTTCCTG | |
| | GCCGACAGACACAGCGGCTGGTGGAGAGCCCCCCTGAGATGGAC | |
| | CCTGAGAGGCAGCCTGGCCCTGAGAAGCCACCTGATGGTGAGAAG | |
| | CAGCCTGAGAAGAAGCAGAAGAAGAAAGCTGAAGCTGGTGGAGGG | |
| | CAGACAC | |
| TB-M4 | GTGGTGGCCGTGACCTACAGCCCCGGCCCCCACCTGGAGAGATTC | SEQ ID |
| | CTGGCTAGCCTGAGCCTGGCCACCGAAAGACCCGTGTCCGTGCTT | NO: 38 |
| | TTGGCCGACAACGGCAGTACGGACGGCACCCCCCAAGCCGCCGT | |
| | GCAGAGATACCCCAACGTGAGACTGCTGCCCACCGGCGCCAACCT | |
| | GGGCTACGGCACCGCCGTGAATAGAACGATAGCTCAGCTGGGCGA | |
| | GATGGCCGGAGACGCCGGTGAACCCTGGGTGGACGATTGGGTCAT | |
| | AGTGGCGAATCCCGACGTGCAATGGGGTCCCGGCAGCATCGATGC | |
| | CCTGCTGGATGCCGCTAGCAGATGGCCCCGCGCTGGTGCCCTGG | |
| | GACCGTTAATTCGGGACCCCGACGGCAGCGTGTACCCTAGCGCTA | |
| | GACAGATGCCTAGCCTGATCAGAGGCGGCATGCACGCCGTGCTGG | |
| | GCCCCTTCTGGCCTAGAAACCCCTGGACCACCGCCTACAGACAAG | |
| | AGAGACTGGAGCCTAGCGAAAGACCCGTGGGCTGGCTGTCGGGCT | |
| | CCTGCCTGCTGGTGAGAAGAAGCGCCTTCGGCCAAGTGGGCGGCT | |
| | TCGACGAGAGATACTTCATGTACATGGAGGACGTGGACCTGGGCG | |
| | ACAGACTGGGCAAGGCCGGCTGGCTAAGCGTTTACGTGCCTAGCG | |
| | CCGAGGTGCTGCACCACAAGGCCCACAGCACCGGCCGGGACCCC | |
| | GCTAGCCACCTGGCCGCCCACCACAAGAGCACCTACATCTTCCTG | |
| | GCCGACAGACACAGCGGCTGGTGGAGAGCCCCCCTGAGATGGAC | |
| | CCTGAGAGGCAGCCTGGCCCTGAGAAGCCACCTGATGGTGAGAAG | |
| | CAGCCTGAGAAGAAGCAGAAGAAGAAAGCTGAAGCTGGTGGAGGG | |
| | CAGACACGGCGGAGGCGGATCTGGCGGAGGCGGCAGCGGCGGC | |
| | GGCGGCAGCGCCGAGATGAAAACCGACGCCGCCACCCTGGCCCA | |
| | AGAGGCCGGCAACTTCGAGAGAATCAGCGGCGACCTCAAGACACA | |
| | GATCGACCAAGTGGAGAGCACGGCTGGCAGCCTGCAAGGGCAATG | |
| | GAGAGGCGCAGCCGGTACTGCCGCACAAGCCGCCGTGGTGAGAT | |
| | TCCAAGAGGCCGCCAACAAGCAGAAGCAAGAGCTGGACGAGATCA | |
| | GCACCAACATCAGACAAGCCGGCGTGCAGTACAGCAGAGCCGACG | |
| | AGGAGCAGCAGCAAGCCCTGAGCTCTCAGATGGGCTTCGGCGGC | |
| | GGCGGCGTGGACTTCGGCGCCCTGCCCCCCGAGATAAACTCCGCT | |
| | AGAATGTACGCCGGCCCGGGAAGCGCTAGCTTGGTCGCAGCCGCA | |
| | CAGATGTGGGACAGCGTGGCTAGCGACCTGTTCAGCGCCGCTAGC | |
| | GCCTTTCAGTCGGTGGTGTGGGGACTTACTGTCGGCAGCTGGATC | |
| | GGCAGTTCGGCTGGCTTGATGGTGGCCGCGGCGTCGCCCTATGTG | |
| | GCCTGGATGAGCGTGACTGCCGGGCAAGCCGAACTGACTGCGGCT | |
| | CAAGTGCGTGTGGCCGCGGCTGCCTATGAAACCGCGTACGGCCTT | |
| | ACAGTTCCGCCTCCCGTGATCGCCGAGAACAGAGCCGAGCTGATG | |
| | ATCCTGATCGCCACCAACCTGCTGGGGCAGAACACCCCCGCCATC | |
| | GCCGTGAACGAGGCCGAGTACGGCGAGATGTGGGCTCAAGATGCT | |
| | GCAGCGATGTTCGGATACGCCGCCGCCACGGCGACCGCCACCGC | |
| | CACCTTACTGCCCTTCGAAGAAGCCCCCGAGATGACAAGCGCCGG | |
| | CGGCTTATTAGAGCAAGCCGCAGCCGTGGAGGAGGCTAGCGACAC | |
| | GGCCGCTGCCAATCAGCTGATGAACAATGTGCCCCAAGCCCTACA | |
| | ACAACTGGCGCAGCCCACCCAAGGCACAACCCCAAGCAGCAAGCT | |
| | GGGCGGCCTGTGGAAAACCGTGAGCCCCCACAGAAGCCCCATCAG | |
| | CAACATGGTGAGCATGGCCAACAACCACATGAGCATGACAAACAGT | |
| | GGCGTGAGCATGACCAATACGCTCAGCAGCATGCTGAAGGGCTTC | |
| | GCGCCGGCCGCCGCTGCACAAGCTGTTCAGACTGCGGCGCAGAA | |
| | CGGGGTGAGAGCAATGAGCAGCCTGGGCTCAAGCCTTGGCAGCTC | |
| | TGGCTTAGGCGGCGGTGTGGCAGCCAACCTGGGCAGAGCCGCCT | |
| | CAGTTGGGAGCCTGTCCGTGCCTCAAGCCTGGGCAGCGGCGAATC | |
| | AAGCTGTCACCCCCGCCGCTAGAGCCCTGCCCCTGACAAGCCTGA | |
| | CTAGTGCGGCAGAGAGAGGCCCCGGCCAAATGCTCGGTGGCTTAC | |
| | CGGTAGGGCAAATGGGGGCAAGAGCGGGAGGCGGTCTTTCCGGC | |
| | GTGCTGAGAGTGCCCCCTAGACCCTACGTGATGCCCCACAGCCCC | |
| | GCCGCCGGCGGCGGAGGCGGATCTGGCGGAGGCGGCAGCGGCG | |
| | GCGGCGGCAGCGTGTCTTTCGTGATGGCCTACCCCGAAATGCTAG | |
| | CCGCCGCCGCAGATACCCTGCAGAGCATCGGCGCAACCACAGTTG | |
| | CAAGCAATGCTGCTGCTGCCGCACCGACTACCGGCGTGGTGCCCC | |
| | CCGCGGCGGATGAGGTGAGCGCCCTAACCGCCGCCCATTTCGCC | |
| | GCACACGCCGCGATGTATCAGAGCGTGAGCGCTAGAGCCGCCGC | |
| | CATCCACGATCAGTTCGTGGCCACCCTGGCTAGCAGCGCTAGCAG | |
| | CTACGCCGCCACCGAGGTGGCCAACGCGGCCGCCGCTAGC | |
| TB-M7 | GTGGACTTCGGCGCCCTGCCCCCCGAGATAAACTCCGCTAGAATG | SEQ ID |
| | TACGCCGGCCCGGGAAGCGCTAGCTTGGTCGCAGCCGCACAGAT | NO: 39 |
| | GTGGGACAGCGTGGCTAGCGACCTGTTCAGCGCCGCTAGCGCCTT | |
| | TCAGTCGGTGGTGTGGGGACTTACTGTCGGCAGCTGGATCGGCAG | |
| | TTCGGCTGGCTTGATGGTGGCCGCGGCGTCGCCCTATGTGGCCTG | |
| | GATGAGCGTGACTGCCGGGCAAGCCGAACTGACTGCGGCTCAAGT | |
| | GCGTGTGGCCGCGGCTGCCTATGAAACCGCGTACGGCCTTACAGT | |
| | TCCGCCTCCCGTGATCGCCGAGAACAGAGCCGAGCTGATGATCCT | |
| | GATCGCCACCAACCTGCTGGGGCAGAACACCCCCGCCATCGCCGT | |
| | GAACGAGGCCGAGTACGGCGAGATGTGGGCTCAAGATGCTGCAGC | |
| | GATGTTCGGATACGCCGCCGCCACGGCGACCGCCACCGCCACCTT | |
| | ACTGCCCTTCGAAGAAGCCCCCGAGATGACAAGCGCCGGCGGCTT | |
| | ATTAGAGCAAGCCGCAGCCGTGGAGGAGGCTAGCGACACGGCCG | |
| | CTGCCAATCAGCTGATGAACAATGTGCCCCAAGCCCTACAACAACT | |
| | GGCGCAGCCCACCCAAGGCACAACCCCAAGCAGCAAGCTGGGCG | |
| | GCCTGTGGAAAACCGTGAGCCCCCACAGAAGCCCCATCAGCAACA | |
| | TGGTGAGCATGGCCAACAACCACATGAGCATGACAAACAGTGGCG | |
| | TGAGCATGACCAATACGCTCAGCAGCATGCTGAAGGGCTTCGCGC | |
| | CGGCCGCCGCTGCACAAGCTGTTCAGACTGCGGCGCAGAACGGG | |
| | GTGAGAGCAATGAGCAGCCTGGGCTCAAGCCTTGGCAGCTCTGGC | |
| | TTAGGCGGCGGTGTGGCAGCCAACCTGGGCAGAGCCGCCTCAGTT | |
| | GGGAGCCTGTCCGTGCCTCAAGCCTGGGCAGCGGCGAATCAAGCT | |
| | GTCACCCCCGCCGCTAGAGCCCTGCCCCTGACAAGCCTGACTAGT | |
| | GCGGCAGAGAGAGGCCCCGGCCAAATGCTCGGTGGCTTACCGGT | |
| | AGGGCAAATGGGGGCAAGAGCGGGAGGCGGTCTTTCCGGCGTGC | |
| | TGAGAGTGCCCCCTAGACCCTACGTGATGCCCCACAGCCCCGCCG | |
| | CCGGCGGCGGCGGCGGCGCCGAGATGAAAACCGACGCCGCCACC | |
| | CTGGCCCAAGAGGCCGGCAACTTCGAGAGAATCAGCGGCGACCTC | |
| | AAGACACAGATCGACCAAGTGGAGAGCACGGCTGGCAGCCTGCAA | |
| | GGGCAATGGAGAGGCGCAGCCGGTACTGCCGCACAAGCCGCCGT | |
| | GGTGAGATTCCAAGAGGCCGCCAACAAGCAGAAGCAAGAGCTGGA | |
| | CGAGATCAGCACCAACATCAGACAAGCCGGCGTGCAGTACAGCAG | |
| | AGCCGACGAGGAGCAGCAGCAAGCCCTGAGCTCTCAGATGGGCTT | |
| | CGGCGGAGGCGGATCTGGCGGAGGCGGCAGCGGCGGCGGCGGC | |
| | AGCGTGTCTTTCGTGATGGCCTACCCCGAAATGCTAGCCGCCGCC | |
| | GCAGATACCCTGCAGAGCATCGGCGCAACCACAGTTGCAAGCAAT | |
| | GCTGCTGCTGCCGCACCGACTACCGGCGTGGTGCCCCCCGCGGC | |
| | GGATGAGGTGAGCGCCCTAACCGCCGCCCATTTCGCCGCACACGC | |
| | CGCGATGTATCAGAGCGTGAGCGCTAGAGCCGCCGCCATCCACGA | |
| | TCAGTTCGTGGCCACCCTGGCTAGCAGCGCTAGCAGCTACGCCGC | |
| | CACCGAGGTGGCCAACGCGGCCGCCGCTAGCGGCGGCGGCGGC | |
| | GTGGTGGCCGTGACCTACAGCCCCGGCCCCCACCTGGAGAGATTC | |
| | CTGGCTAGCCTGAGCCTGGCCACCGAAAGACCCGTGTCCGTGCTT | |
| | TTGGCCGACAACGGCAGTACGGACGGCACCCCCCAAGCCGCCGT | |
| | GCAGAGATACCCCAACGTGAGACTGCTGCCCACCGGCGCCAACCT | |
| | GGGCTACGGCACCGCCGTGAATAGAACGATAGCTCAGCTGGGCGA | |
| | GATGGCCGGAGACGCCGGTGAACCCTGGGTGGACGATTGGGTCAT | |
| | AGTGGCGAATCCCGACGTGCAATGGGGTCCCGGCAGCATCGATGC | |
| | CCTGCTGGATGCCGCTAGCAGATGGCCCCGCGCTGGTGCCCTGG | |
| | GACCGTTAATTCGGGACCCCGACGGCAGCGTGTACCCTAGCGCTA | |
| | GACAGATGCCTAGCCTGATCAGAGGCGGCATGCACGCCGTGCTGG | |
| | GCCCCTTCTGGCCTAGAAACCCCTGGACCACCGCCTACAGACAAG | |
| | AGAGACTGGAGCCTAGCGAAAGACCCGTGGGCTGGCTGTCGGGCT | |
| | CCTGCCTGCTGGTGAGAAGAAGCGCCTTCGGCCAAGTGGGGGGCT | |
| | TCGACGAGAGATACTTCATGTACATGGAGGACGTGGACCTGGGCG | |
| | ACAGACTGGGCAAGGCCGGCTGGCTAAGCGTTTACGTGCCTAGCG | |
| | CCGAGGTGCTGCACCACAAGGCCCACAGCACCGGCCGGGACCCC | |
| | GCTAGCCACCTGGCCGCCCACCACAAGAGCACCTACATCTTCCTG | |
| | GCCGACAGACACAGCGGCTGGTGGAGAGCCCCCCTGAGATGGAC | |
| | CCTGAGAGGCAGCCTGGCCCTGAGAAGCCACCTGATGGTGAGAAG | |
| | CAGCCTGAGAAGAAGCAGAAGAAGAAAGCTGAAGCTGGTGGAGGG | |
| | CAGACAC | |
| TB-M8 | GCCGAGATGAAAACCGACGCCGCCACCCTGGCCCAAGAGGCCGG | SEQ ID |
| | CAACTTCGAGAGAATCAGCGGCGACCTCAAGACACAGATCGACCA | NO: 40 |
| | AGTGGAGAGCACGGCTGGCAGCCTGCAAGGGCAATGGAGAGGCG | |
| | CAGCCGGTACTGCCGCACAAGCCGCCGTGGTGAGATTCCAAGAGG | |
| | CCGCCAACAAGCAGAAGCAAGAGCTGGACGAGATCAGCACCAACA | |
| | TCAGACAAGCCGGCGTGCAGTACAGCAGAGCCGACGAGGAGCAG | |
| | CAGCAAGCCCTGAGCTCTCAGATGGGCTTCGGCGGCGGCGGCGT | |
| | GGTGGCCGTGACCTACAGCCCCGGCCCCCACCTGGAGAGATTCCT | |
| | GGCTAGCCTGAGCCTGGCCACCGAAAGACCCGTGTCCGTGCTTTT | |
| | GGCCGACAACGGCAGTACGGACGGCACCCCCCAAGCCGCCGTGC | |
| | AGAGATACCCCAACGTGAGACTGCTGCCCACCGGCGCCAACCTGG | |
| | GCTACGGCACCGCCGTGAATAGAACGATAGCTCAGCTGGGCGAGA | |
| | TGGCCGGAGACGCCGGTGAACCCTGGGTGGACGATTGGGTCATAG | |
| | TGGCGAATCCCGACGTGCAATGGGGTCCCGGCAGCATCGATGCCC | |
| | TGCTGGATGCCGCTAGCAGATGGCCCCGCGCTGGTGCCCTGGGA | |
| | CCGTTAATTCGGGACCCCGACGGCAGCGTGTACCCTAGCGCTAGA | |
| | CAGATGCCTAGCCTGATCAGAGGCGGCATGCACGCCGTGCTGGGC | |
| | CCCTTCTGGCCTAGAAACCCCTGGACCACCGCCTACAGACAAGAG | |
| | AGACTGGAGCCTAGCGAAAGACCCGTGGGCTGGCTGTCGGGCTCC | |
| | TGCCTGCTGGTGAGAAGAAGCGCCTTCGGCCAAGTGGGCGGCTTC | |
| | GACGAGAGATACTTCATGTACATGGAGGACGTGGACCTGGGCGAC | |
| | AGACTGGGCAAGGCCGGCTGGCTAAGCGTTTACGTGCCTAGCGCC | |
| | GAGGTGCTGCACCACAAGGCCCACAGCACCGGCCGGGACCCCGC | |
| | TAGCCACCTGGCCGCCCACCACAAGAGCACCTACATCTTCCTGGC | |
| | CGACAGACACAGCGGCTGGTGGAGAGCCCCCCTGAGATGGACCCT | |
| | GAGAGGCAGCCTGGCCCTGAGAAGCCACCTGATGGTGAGAAGCAG | |
| | CCTGAGAAGAAGCAGAAGAAGAAAGCTGAAGCTGGTGGAGGGCAG | |
| | ACACGGAAGCGGAGCTACTAACTTCAGCCTGCTGAAGCAGGCTGG | |
| | GGACGTGGAGGAGAACCCTGGACCTATGGTGGACTTCGGCGCCCT | |
| | GCCCCCCGAGATAAACTCCGCTAGAATGTACGCCGGCCCGGGAAG | |
| | CGCTAGCTTGGTCGCAGCCGCACAGATGTGGGACAGCGTGGCTAG | |
| | CGACCTGTTCAGCGCCGCTAGCGCCTTTCAGTCGGTGGTGTGGGG | |
| | ACTTACTGTCGGCAGCTGGATCGGCAGTTCGGCTGGCTTGATGGT | |
| | GGCCGCGGCGTCGCCCTATGTGGCCTGGATGAGCGTGACTGCCG | |
| | GGCAAGCCGAACTGACTGCGGCTCAAGTGCGTGTGGCCGCGGCT | |
| | GCCTATGAAACCGCGTACGGCCTTACAGTTCCGCCTCCCGTGATC | |
| | GCCGAGAACAGAGCCGAGCTGATGATCCTGATCGCCACCAACCTG | |
| | CTGGGGCAGAACACCCCCGCCATCGCCGTGAACGAGGCCGAGTA | |
| | CGGCGAGATGTGGGCTCAAGATGCTGCAGCGATGTTCGGATACGC | |
| | CGCCGCCACGGCGACCGCCACCGCCACCTTACTGCCCTTCGAAGA | |
| | AGCCCCCGAGATGACAAGCGCCGGCGGCTTATTAGAGCAAGCCGC | |
| | AGCCGTGGAGGAGGCTAGCGACACGGCCGCTGCCAATCAGCTGAT | |
| | GAACAATGTGCCCCAAGCCCTACAACAACTGGCGCAGCCCACCCA | |
| | AGGCACAACCCCAAGCAGCAAGCTGGGCGGCCTGTGGAAAACCGT | |
| | GAGCCCCCACAGAAGCCCCATCAGCAACATGGTGAGCATGGCCAA | |
| | CAACCACATGAGCATGACAAACAGTGGCGTGAGCATGACCAATACG | |
| | CTCAGCAGCATGCTGAAGGGCTTCGCGCCGGCCGCCGCTGCACAA | |
| | GCTGTTCAGACTGCGGCGCAGAACGGGGTGAGAGCAATGAGCAGC | |
| | CTGGGCTCAAGCCTTGGCAGCTCTGGCTTAGGCGGCGGTGTGGCA | |
| | GCCAACCTGGGCAGAGCCGCCTCAGTTGGGAGCCTGTCCGTGCCT | |
| | CAAGCCTGGGCAGCGGCGAATCAAGCTGTCACCCCCGCCGCTAGA | |
| | GCCCTGCCCCTGACAAGCCTGACTAGTGCGGCAGAGAGAGGCCCC | |
| | GGCCAAATGCTCGGTGGCTTACCGGTAGGGCAAATGGGGGCAAGA | |
| | GCGGGAGGCGGTCTTTCCGGCGTGCTGAGAGTGCCCCCTAGACC | |
| | CTACGTGATGCCCCACAGCCCCGCCGCCGGGGGGGGCGGCGGCG | |
| | TGTCTTTCGTGATGGCCTACCCCGAAATGCTAGCCGCCGCCGCAG | |
| | ATACCCTGCAGAGCATCGGCGCAACCACAGTTGCAAGCAATGCTG | |
| | CTGCTGCCGCACCGACTACCGGCGTGGTGCCCCCCGCGGCGGAT | |
| | GAGGTGAGCGCCCTAACCGCCGCCCATTTCGCCGCACACGCCGC | |
| | GATGTATCAGAGCGTGAGCGCTAGAGCCGCCGCCATCCACGATCA | |
| | GTTCGTGGCCACCCTGGCTAGCAGCGCTAGCAGCTACGCCGCCAC | |
| | CGAGGTGGCCAACGCGGCCGCCGCTAGC | |

*Any of the sequences in Table 6 may include a methionine residue at the start.
**Any of the sequences in Table 7 may include a start codon (ATG) at the 5′ end of the construct. Further, any of the sequences may include a stop codon selected from TAA, TAG, or TGA at the 3′ end of the construct. In particular, the inventors of the present invention have designed a specific stop codon sequence, comprising four consecutive stop codons with the nucleotide sequence of TGATAATGATAG (SEQ ID NO: 32).

Purification and Analysis of In Vitro Transcribed mRNA

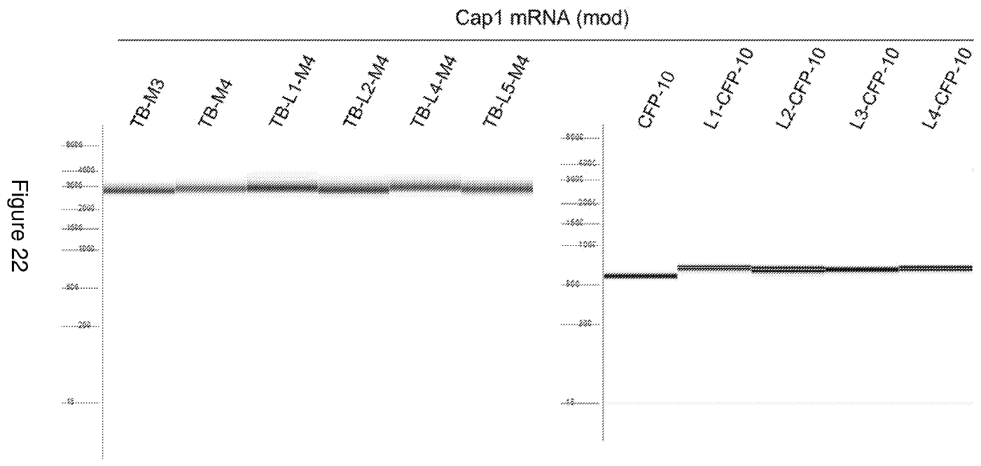

The size and integrity of the exemplary in vitro transcribed TB antigen-encoding CFP-10 monogenic mRNA with leader sequences, polygenic mRNA, and polygenic mRNA with leader sequences were analysed using capillary gel electrophoresis on the 5200 Fragment Analyzer using the DNF-471 RNA kit (15 nt) (Agilent). As shown in FIG. 21, in vitro transcribed mRNAs were successfully obtained for the longer polyprotein transcripts, TB-M3 and TB-M4, and for transcripts that contain leader sequences. FIG. 22 shows capillary gel images depicting the size and integrity of TB-M3 and TB-M4 polygenic mRNA transcripts with and without leader sequences (L1, L2, L3, L4, L5) and CFP-10 monogenic mRNA transcripts with and without leader sequences (L1, L2, L3, L4).

### Formulation of Lipid Nanoparticles (LNPs)

Polygenic mRNA was incorporated into LNP's as described above. LNPs for the polygenic mRNAs ranged from 68.12 nm-100.7 nm in size.

### Example 7

### Induction of Cellular Immune Responses to Polygenic M. Tb Antigen Vaccines

### Immunization of Mice and Preparation of Splenocytes

All animal work was carried out in accordance with protocols approved by the University of the Witwatersrand Animal Ethics Screening Committee. On day zero (DO), female BALB/c, C57BL6 and C3HeB/FeJ mice were injected intramuscularly with lipid-nanoparticle formulated TB polyantigen-expressing mRNA vaccines TB-M3, TB-M4 and TB-M7. Control groups were injected intramuscularly with saline (50 μl/mouse per vaccination) or a lipid-nanoparticle formulated control mRNA encoding Firefly Luciferase (10 μg/mouse in a total volume of 50 μl per vaccination). Mice received boost vaccinations three weeks later. Serum was collected directly before vaccinations (day zero and week 3) and at week 5. Spleens were collected at week 5 for analysis of antigen-specific T cells. Table 8 below shows that Animal groups used for the polygenic mRNA vaccines and the vaccine doses administered.

TABLE 8
Animal groups and doses for polyprotein TB mRNA vaccines.

| | | | | Vaccine dose | |
|---|---|---|---|---|---|
| | | Number of | | (per dose) | |
| Group | Strain | mice | Vaccine | 2-dose series | |
| 1 | BALB/c | 3 | Saline | 50 | μl |
| 2 | BALB/c | 3 | Control mRNA | 10 | μg |
| 3 | BALB/c | 4 | TB-M3 mRNA | 10 | μg |
| 4 | BALB/c | 4 | TB-M4 mRNA | 10 | μg |
| 5 | BALB/c | 4 | TB-M7 mRNA | 10 | μg |
| 6 | C57BL/6 | 2 | Saline | 50 | μl |
| 7 | C57BL/6 | 3 | Control mRNA | 10 | μg |
| 8 | C57BL/6 | 4 | TB-M3 mRNA | 10 | μg |
| 9 | C57BL/6 | 4 | TB-M4 mRNA | 10 | μg |
| 10 | C57BL/6 | 4 | TB-M7 mRNA | 10 | μg |
| 11 | C3HeB/FeJ | 3 | Saline | 50 | μl |
| 12 | C3HeB/FeJ | 3 | Control mRNA | 10 | μg |
| 13 | C3HeB/FeJ | 4 | TB-M3 mRNA | 10 | μg |
| 14 | C3HeB/FeJ | 4 | TB-M4 mRNA | 10 | μg |
| 15 | C3HeB/FeJ | 4 | TB-M7 mRNA | 10 | μg |
| 16 | BALB/c | 4 | TB-L1-M4 | 10 | μg |
| 17 | BALB/c | 3 | TB-L2-M4 | 10 | μg |
| 18 | BALB/c | 3 | TB-L4-M4 | 10 | μg |
| 19 | BALB/c | 3 | TB-L5-M4 | 10 | μg |
| 20 | BALB/c | 4 | TB-L5-M3 | 10 | μg |
| 21 | C57BL/6 | 4 | TB-L1-M4 | 10 | μg |
| 22 | C57BL/6 | 3 | TB-L2-M4 | 10 | μg |
| 23 | C57BL/6 | 3 | TB-L4-M4 | 10 | μg |
| 24 | C57BL/6 | 3 | TB-L5-M4 | 10 | μg |
| 25 | C57BL/6 | 4 | TB-L5-M3 | 10 | μg |
| 26 | C3HeB/FeJ | 4 | TB-L1-M4 | 10 | μg |
| 27 | C3HeB/FeJ | 2 | TB-L2-M4 | 10 | μg |
| 28 | C3HeB/FeJ | 2 | TB-L4-M4 | 10 | μg |
| 29 | C3HeB/FeJ | 2 | TB-L5-M4 | 10 | μg |
| 30 | C3HeB/FeJ | 4 | TB-L5-M3 | 10 | μg |

Splenocytes were isolated from mice according to standard protocols as described in Example 5.

### T-Cell Stimulations, Quantification of Secreted Cytokines, and Intracellular Cytokine Staining.

Peptide pools covering the M.tb mRNA vaccine antigens CFP-10, WbbL1, PE-13 and PPE-18, a mix of all four antigens (peptide mix CWPP), and M.tb non-vaccine antigens TB10.4, were used for T cell stimulations. Cell stimulations and processing of samples from vaccinated mice for quantification of secreted cytokines and intracellular cytokine staining was performed according to standard protocols as described in Example 5.

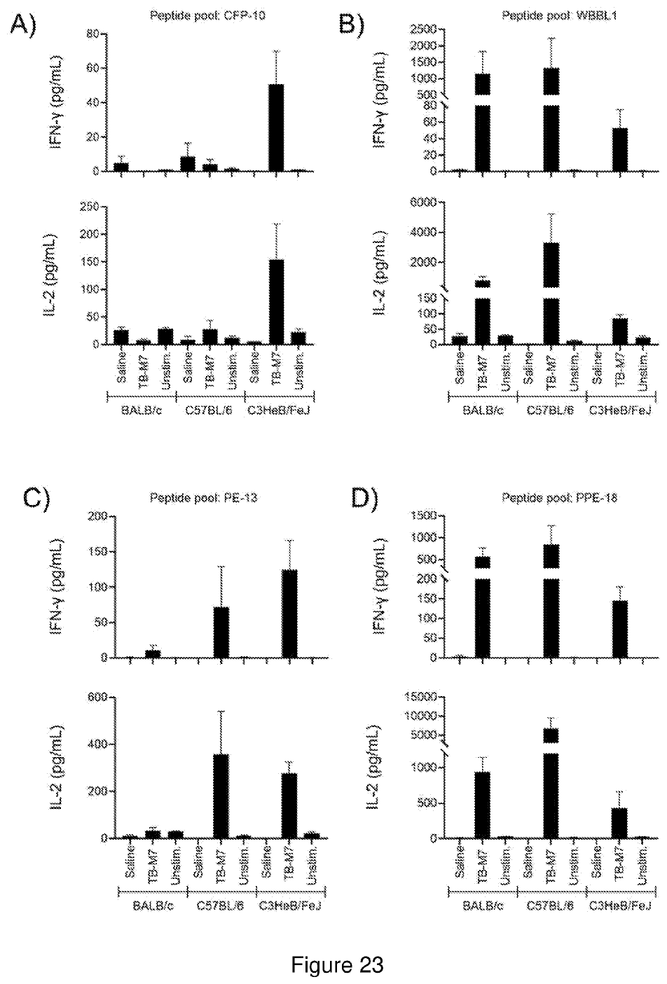

Secreted cytokine profiles in ex vivo-stimulated spleen cells from mice injected intramuscularly with saline or immunized with 10 μg of LNP-formulated control mRNA encoding a bioluminescence reporter gene, or 10 μg of LNP-formulated polygenic TB-M7 mRNA are shown in FIG. 23. Exemplary cytokine profiles for IFN-γ and IL-2 are shown for BALB/c, C57BL6 and C3HeB/FeJ mice. Unstimulated spleen cells from TB-M7 mRNA vaccinated mice were included as a control. These results confirm individual antigen-specific T cell activation of all four TB antigens included in the polyprotein. Specifically, stimulation with CFP-10 peptide pools showed increased concentrations of IFN-γ and IL-2 in C3HeB/FeJ mice, stimulation with Wbbl1 peptide pools showed increased concentrations of IFN-γ and IL-2 in BALB/c, C57BL6 and C3HeB/FeJ mice, stimulation with PE-13 peptide pools showed increased concentrations of IFN-γ and IL-2 in C57BL6 and C3HeB/FeJ mice and stimulation with PPE-18 peptide pools show increased concentrations of IFN-γ and IL-2 in BALB/c, C57BL6 and C3HeB/FeJ mice.

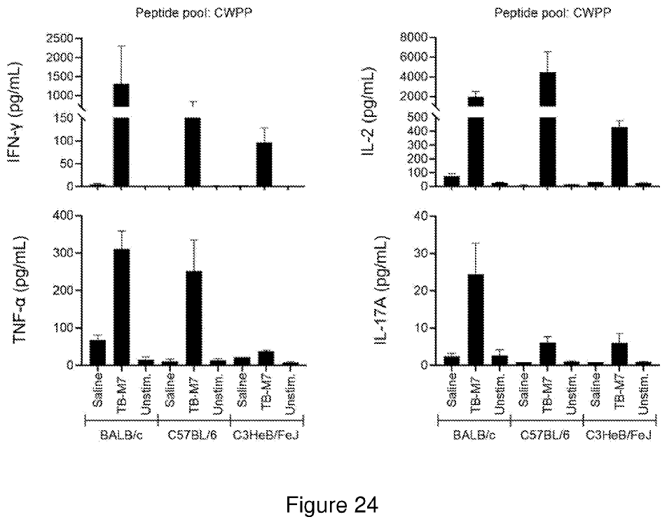

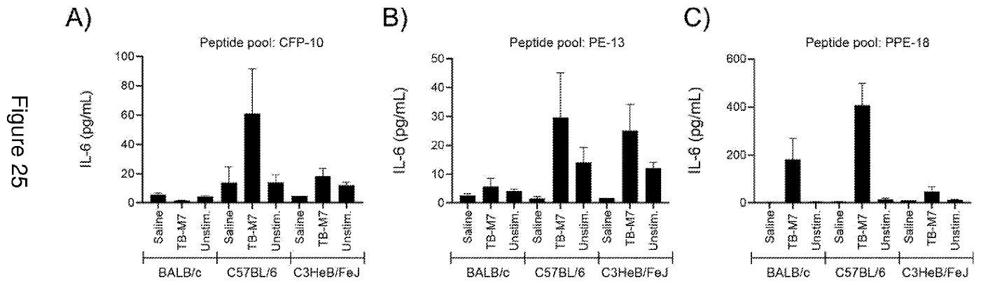

FIG. 24 shows exemplary secreted IFN-γ, IL-2, TNF-α and IL-17A cytokine profiles in ex vivo-stimulated spleen cells from BALB/c, C57BL6 and C3HeB/FeJ mice stimulated with mixed antigen peptide pool (Peptide pool: CWPP) following vaccination with the TB-M7 polyprotein mRNA. BALB/c, C57BL6 and C3HeB/FeJ mice showed increased IFN-γ and IL-2 following TB-M7 mRNA vaccination. Additionally, BALB/c mice showed increased TNF-α and IL-17A, and C57BL6 mice show increased TNF-α.

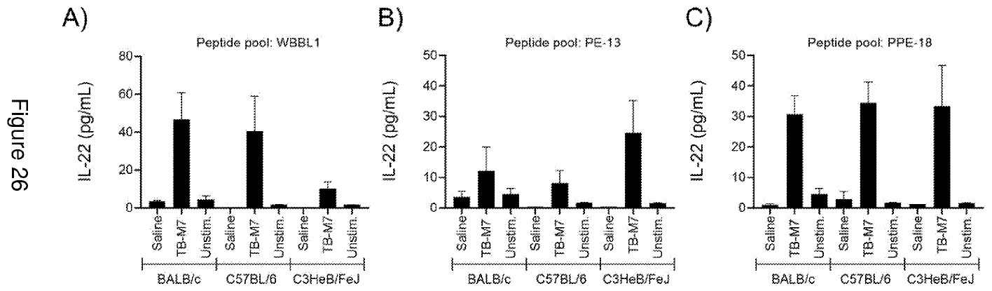

For spleen cells stimulated with CFP-10, PE-13 and PPE-18 peptide pools, secreted IL-6 concentrations in C57BL6 mice vaccinated with TB-M7 polyprotein mRNA were increased for all three peptide pools (FIG. 25). In addition, BALB/c mice showed increased IL-6 following stimulation with PPE-18 peptide pool.

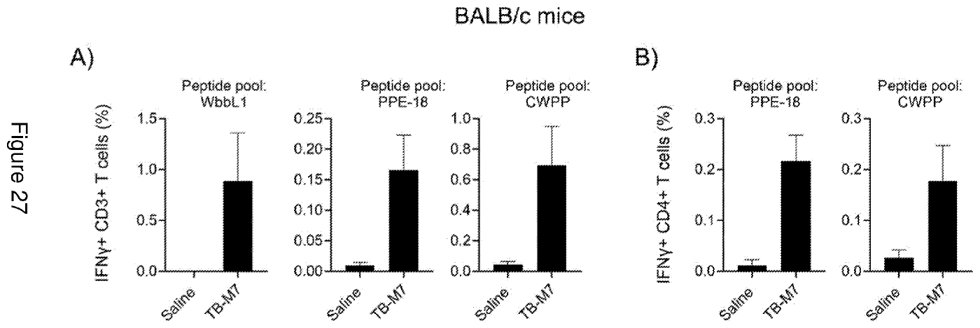

Secreted IL-22 concentrations were increased in BALB/c, C57BL6 and C3HeB/FeJ mice vaccinated with TB-M7 polyprotein mRNA for spleen cells stimulated with each of the WBBL1, PE-13 and PPE-18 peptide pools (FIG. 26).

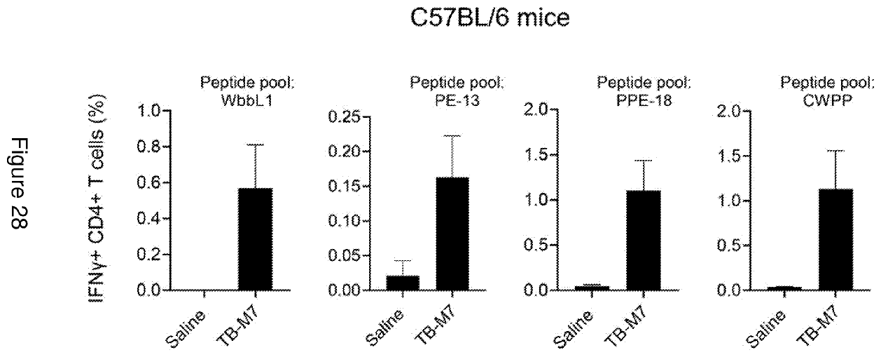

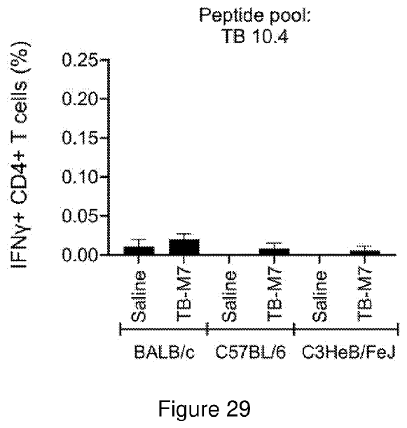

As can be seen from FIG. 27, the percentage of IFN-γ +CD3+ T cells increased following cell stimulation with WbbL1, PPE-18 and CWPP peptide pools in BALB/c mice vaccinated with TB-M7 polyprotein mRNA. PPE-18 specific IFN-γ +CD4+ T cells were observed. Additionally, the percentage of IFN-γ +CD4+ T cells increased in mice vaccinated with TB-M7 polyprotein mRNA following cell stimulation with WbbL1, PE-13, PPE-18 and CWPP peptide pools in C57BL6 mice (FIG. 28). Further, flow cytometry-based detection of IFN-γ positive CD4 T-cells shows no activation of IFN-γ in ex-vivo stimulation of spleen cells from vaccinated BALB/c, C57BL6 and C3HeB/FeJ mice with a TB antigen (TB10.4) not included in the polyprotein (FIG. 29). This confirms the abovementioned results depicting antigen-specific T-cell stimulation.

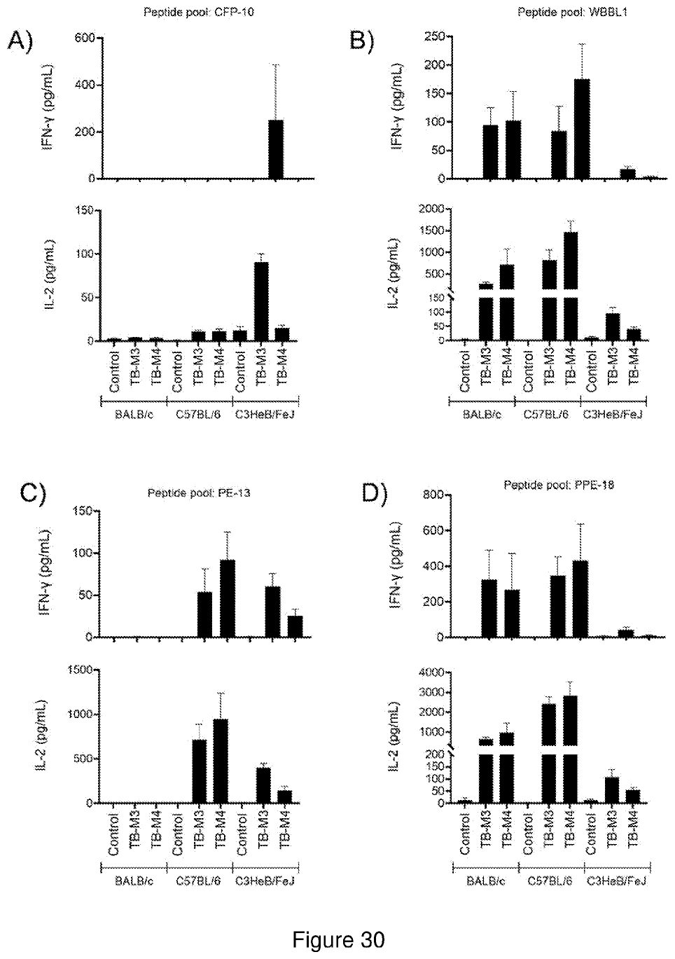

mRNA vaccines encoding TB-M3 and TB-M4 polyprotein were also investigated as described in Table 8 above. Secreted cytokine profiles in ex vivo-stimulated spleen cells from mice injected intramuscularly with 10 μg of LNP-formulated control mRNA encoding a bioluminescence reporter gene (control), or 10 μg of LNP-formulated polygenic TB-M3 or TB-M4 mRNA were anlaysed. Exemplary cytokine profiles for IFN-γ and IL-2 are shown in FIG. 30 for BALB/c, C57BL6 and C3HeB/FeJ mice. Stimulation with CFP-10 peptide pools show increased concentrations of IFN-γ and IL-2 C3HeB/FeJ mice. Stimulation with Wbbl1 peptide pools show increased concentrations of IFN-γ in and IL-2 in BALB/c, C57BL6 and C3HeB/FeJ mice. Stimulation with PE-13 peptide pools show increased concentrations of IFN-γ and IL-2 in C57BL6 and C3HeB/FeJ mice. Finally, stimulation with PPE-18 peptide pools show increased concentrations of IFN-γ and IL-2 in BALB/c and C57BL6 mice.

These results confirm individual antigen-specific T cell activation of all four TB antigens included in the polyprotein and that the polyprotein mRNA vaccines are immunogenic in mice.

### REFERENCES

Han, A., Glanville, J., Hansmann, L. & Davis, M. M. Linking T-cell receptor sequence to functional phenotype at the single-cell level. Nat. Biotechnol. 32, 684-692 (2014).
Glanville, J. et al. Identifying specificity groups in the T cell receptor repertoire. Nature 547. (2017).
Huang, H., Wang, C., Rubelt, F., Scriba, T. J. & Davis, M. M. Analyzing the Mycobacterium tuberculosis immune response by T-cell receptor clustering with GLIPH2 and genome-wide antigen screening. Nat. Biotechnol. (2020).

1. A vaccine composition comprising:
a. a nucleic acid encoding a Wbbl1 antigen having an amino acid sequence which has at least 80% sequence identity to SEQ ID NO:1;
b. a nucleic acid encoding a CFP-10 antigen having an amino acid sequence which has at least 80% sequence identity to SEQ ID NO: 2;
C. a nucleic acid encoding a PPE18 antigen having an amino acid sequence of which has at least 80% sequence identity to SEQ ID NO: 3; and
d. a nucleic acid encoding a PE13 antigen having an amino acid sequence which has at least 80% sequence identity to SEQ ID NO: 4,
wherein the nucleic acids are provided on one or more nucleic acid constructs.

2. The vaccine composition of claim 1, wherein:
(a) the nucleic acid encoding the Wbbl1 antigen has at least about 90%, at least about 95%, at least about 96%, at least about 97%, at least about 98% or at least about 99% sequence identity to SEQ ID NO: 1, or has the amino acid sequence of SEQ ID NO: 1;
(b) the nucleic acid encoding the CFP-10 antigen has at least about 90%, at least about 95%, at least about 96%, at least about 97%, at least about 98% or at least about 99% sequence identity to SEQ ID NO: 2, or has the amino acid sequence of SEQ ID NO: 2;
(c) the nucleic acid encoding the PPE18 antigen has at least about 90%, at least about 95%, at least about 96%, at least about 97%, at least about 98% or at least about 99% sequence identity to SEQ ID NO: 3, or has the amino acid sequence of SEQ ID NO: 3; and/or
(d) the nucleic acid encoding the PE13 antigen has at least about 90%, at least about 95%, at least about 96%, at least about 97%, at least about 98% or at least about 99% sequence identity to SEQ ID NO: 4, or has the amino acid sequence of SEQ ID NO: 4.

3. (canceled)

4. The vaccine composition of claim 1, wherein (a) has a nucleotide sequence having at least 80% sequence identity to SEQ ID NO: 5, (b) has a nucleotide sequence having at least 80% sequence identity to SEQ ID NO: 6, (c) has a nucleotide sequence having at least 80% sequence identity to SEQ ID NO: 7, and (d) has a nucleotide sequence having at least 80% sequence identity to SEQ ID NO: 8.

5. The vaccine composition of claim 4, wherein:
the nucleotide sequence of (a) has at least about 90%, at least about 95%, at least about 96%, at least about 97%, at least about 98% or at least about 99% sequence identity to SEQ ID NO: 5 or is identical to SEQ ID NO: 5;
the nucleotide sequence of (b) has at least about 90%, at least about 95%, at least about 96%, at least about 97%, at least about 98% or at least about 99% sequence identity to SEQ ID NO: 6 or is identical to SEQ ID NO: 6;
the nucleotide sequence of (c) has at least about 90%, at least about 95%, at least about 96%, at least about 97%, at least about 98% or at least about 99% sequence identity to SEQ ID NO: 7 or is identical to SEQ ID NO: 7; and/or
the nucleotide sequence of (d) has at least about 90%, at least about 95%, at least about 96%, at least about 97%, at least about 98% or at least about 99% sequence identity to SEQ ID NO: 8 or is identical to SEQ ID NO: 8.

6. The vaccine composition of claim 1, wherein at least one or all of the nucleic acids further comprises a leader nucleotide sequence encoding a secretory peptide signal.

7. (canceled)

8. The vaccine composition of claim 6, wherein each secretory peptide signal is:
a) independently selected from the group consisting of SEQ ID NO: 20, SEQ ID NO: 22, SEQ ID NO: 24, SEQ ID NO: 26, and SEQ ID NO: 28; or
b) encoded by a nucleotide sequence at least about 90% identical to the sequence set forth in the group consisting of SEQ ID NO: 21, SEQ ID NO: 23, SEQ ID NO: 25, SEQ ID NO: 27, and SEQ ID NO: 29.

9. (canceled)

10. The vaccine composition of claim 1, wherein the nucleic acid sequences are provided on a single nucleic acid construct,
wherein each of the nucleic acids is separated by a nucleotide sequence encoding a linker, each linker being independently selected from the group consisting of the linker sequences set forth in any one of SEQ ID NOs: 9-19.

11-13. (canceled)

14. The vaccine composition of claim 10, wherein the nucleic acid construct encodes a polyprotein having an amino acid sequence of any one of SEQ ID NOs: 33-36.

15. The vaccine composition of claim 10, wherein the nucleic acid construct has a nucleotide sequence substantially identical to any one of SEQ ID NOs: 37-40.

16. The vaccine composition of claim 1, wherein the nucleic acid is mRNA.

17. (canceled)

18. The vaccine composition of claim 16, wherein the mRNA includes one or more modified nucleotides selected from N1-methyl-pseudouridine and pseudouridine.

19. (canceled)

20. The vaccine composition of claim 16, wherein the mRNA is comprised in a lipid nanoparticle.

21. The vaccine composition of claim 1, wherein the vaccine composition is capable of eliciting a protective immune response against Mycobacterium tuberculosis.

22. (canceled)

23. A method of eliciting a protective immune response against Mycobacterium tuberculosis in a subject, the method comprising administering the vaccine composition of to the subject.

24. (canceled)

25. A nucleic acid construct encoding a Wbbl1 antigen, a CFP-10 antigen, a PPE18 antigen and a PE13 antigen.

26. (canceled)

27. The nucleic acid construct of claim 25, which is a mRNA construct.

28-30. (canceled)

31. The nucleic acid construct of claim 25, wherein:
(a) the Wbbl1 antigen has at least about 80%, at least about 90%, at least about 95%, at least about 96%, at least about 97%, at least about 98% or at least about 99% sequence identity to SEQ ID NO: 1, or has the amino acid sequence of SEQ ID NO: 1;
(b) the CFP-10 antigen has at least about 80%, at least about 90%, at least about 95%, at least about 96%, at least about 97%, at least about 98% or at least about 99% sequence identity to SEQ ID NO: 2, or has the amino acid sequence of SEQ ID NO: 2;
(c) the PPE18 antigen has at least about 80%, at least about 90%, at least about 95%, at least about 96%, at least about 97%, at least about 98% or at least about 99% sequence identity to SEQ ID NO: 3, or has the amino acid sequence of SEQ ID NO: 3; and/or
(d) the PE13 antigen has at least about 80%, at least about 90%, at least about 95%, at least about 96%, at least about 97%, at least about 98% or at least about 99% sequence identity to SEQ ID NO: 4, or has the amino acid sequence of SEQ ID NO: 4.

32. The nucleic acid construct of claim 25, wherein:
(a) the Wbbl1 antigen is encoded by a nucleotide sequence having at least 80%, at least about 90%, at least about 95%, at least about 96%, at least about 97%, at least about 98% or at least about 99% sequence identity to SEQ ID NO: 5;
(b) the CFP-10 antigen is encoded by a nucleotide sequence having at least 80%, at least about 90%, at least about 95%, at least about 96%, at least about 97%, at least about 98% or at least about 99% sequence identity to SEQ ID NO: 6;
(c) the PPE18 antigen is encoded by a nucleotide sequence having at least 80%, at least about 90%, at least about 95%, at least about 96%, at least about 97%, at least about 98% or at least about 99% sequence identity to SEQ ID NO: 7; and
(d) the PE13 antigen is encoded by a nucleotide sequence having at least 80%, at least about 90%, at least about 95%, at least about 96%, at least about 97%, at least about 98% or at least about 99% sequence identity to SEQ ID NO: 8.

33. The nucleic acid construct of claim 25, which encodes a polyprotein having an amino acid sequence of any one of SEQ ID NOs: 33-36.

34. The nucleic acid construct of claim 25, which has a nucleotide sequence which has at least 80%, at least about 90%, at least about 95%, at least about 96%, at least about 97%, at least about 98% or at least about 99% sequence identity to any one of SEQ ID NOS: 37-40.
